(19) United States
(12) Patent Application Publication
(10) Pub. No.: US 2022/0154295 A1
(43) Pub. Date: May 19, 2022

(54) METHODS AND MATERIALS FOR ASSESSING AND TREATING CANCER

(71) Applicant: The Johns Hopkins University, Baltimore, MD (US)

(72) Inventors: Victor Velculescu, Baltimore, MD (US); Valsamo Anagnostou, Baltimore, MD (US); Noushin Niknafs Kermani, Baltimore, MD (US)

(21) Appl. No.: 17/598,690

(22) PCT Filed: Mar. 27, 2020
(86) PCT No.: PCT/US2020/025551

§ 371 Date: Sep. 27, 2021

Related U.S. Application Data
(60) Provisional application No. 62/824,807, filed on Mar. 27, 2019.

(51) Int. Cl. C12Q 1/6886; A61P 35/00; A61K 45/06; A61K 39/395; C07K 16/28

(52) U.S. Cl. CPC C12Q 1/6886; A61P 35/00; A61K 45/06; A61K 39/3955; A61K 2039/505; C12Q 2600/156; C12Q 2600/16; C12Q 2600/106; C07K 16/2818

(57) ABSTRACT

This document relates to methods and materials involved in assessing and/or treating a mammal having a cancer. For example, methods and materials provided herein can be used to determine the corrected tumor mutation burden (cTMB) of one or more cells (e.g., one or more cancer cells) from a mammal having cancer, thereby identifying the cancer as being likely to respond to a particular cancer treatment (e.g., a cancer immunotherapy). This document also provides methods and materials for treating a mammal identified as having a cancer likely to respond to a particular cancer treatment.

### PRIORITY CLAIM

This application claims benefits of priority to U.S. Provisional Application No. 62/824,807 filed Mar. 27, 2019, the entire contents of which are incorporated herein by reference.

### STATEMENT REGARDING FEDERAL FUNDING

This invention was made with government support under CA180950, CA006973, and CA121113 awarded by the National Institutes of Health. The government has certain rights in the invention.

### BACKGROUND

### 1. Technical Field

This document relates to methods and materials involved in assessing and/or treating a mammal having a cancer. For example, methods and materials provided herein can be used to determine the corrected tumor mutation burden (cTMB) of one or more cells (e.g., one or more cancer cells) from a mammal having cancer, thereby identifying the cancer as being likely to respond to a particular cancer treatment (e.g., a cancer immunotherapy). This document also provides methods and materials for treating a mammal identified as having a cancer likely to respond to a particular cancer treatment.

### 2. Background Information

A high tumor mutation burden (TMB) has been associated with benefit from immune checkpoint blockade (ICB) across tumor types (Yarchoan et al., The New England J. Med. 377:2500-2501 (2017); and Samstein et al., Nature genetics, doi:10.1038/s41588-018-0312-8 (2019)). Despite the value of TMB in predicting response and survival to ICB, there are tumors with a high TMB that do not respond and conversely there are tumors with low TMB that benefit from immunotherapy. Moreover, tissue-based TMB estimates may be challenging in low tumor purity samples and in tumors with a higher intra-tumoral heterogeneity. These limitations are reflected in the current NCCN guidelines, where the use of TMB as a predictive biomarker is limited by lack of calibration and harmonization across multiple next-generation sequencing platforms. Furthermore, response to immunotherapy is orchestrated by immune-related pathways, with the antigen presentation machinery playing a major role as mutation-associated neo-antigens (MANAs) are presented on MHC-I molecules to CD8+ T cells and trigger an anti-tumor immune response that translates to clinical benefit. Genetic variation in the antigen presenting machinery, both at a germline as well as a somatic level may therefore modulate an effective anti-tumor immune response (Gettinger et al., Cancer discovery 7:1420-1435 (2017); and Chowell et al., Science 359:582-587 (2018)).

### SUMMARY

This document provides methods and materials for assessing and/or treating a mammal having a cancer. For example, methods and materials provided herein can be used to determine the cTMB of one or more cells (e.g., one or more cancer cells) from a mammal having cancer, thereby identifying the cancer as being likely to respond to a particular cancer treatment (e.g., a cancer immunotherapy). This document also provides methods and materials for treating a mammal identified as having a cancer likely to respond to a particular cancer treatment.

As demonstrated herein, TMB can be corrected for tumor purity to obtain a cTMB which can be used to more accurately predict a patient outcome for immune checkpoint blockade. Furthermore, cTMB can be combined with genomic alterations in receptor tyrosine kinase (RTK) genes, genome-wide mutational signatures, and HLA class I genetic variation to capture the multifaceted nature of the tumor-immune system crosstalk to more accurately predict a patient outcome for immune checkpoint blockade. For example, this document demonstrates that an analysis of whole exome sequence data from 3,788 TCGA tumor samples found a significant correlation between TMB and tumor purity, suggesting that samples with low tumor purity are likely to have inaccurate TMB estimates. Whole exome sequencing using tumor samples from a cohort of 104 non-small cell lung cancer patients treated with immune checkpoint blockade identified improved markers of response, which were validated in a second independent cohort of immunotherapy treated lung cancer patients.

Having the ability to more accurately predict whether a patient is likely to respond to a particular cancer treatment (e.g., a cancer immunotherapy) can allow clinicians to provide an individualized approach in selected cancer treatments, thereby improving disease-free survival and/or overall survival and/or minimizing subjecting patients to ineffective treatments. In addition, insights into new mechanisms of resistance to immune checkpoint blockade described herein can lay the groundwork for the identification of molecular markers of response to a particular cancer treatment.

In general, one aspect of this document features methods for treating mammals having cancer where the methods can include, or consist essentially of, identifying a sample from a mammal as having a mutation in an ARID1A nucleic acid sequence; and administering a cancer immunotherapy to the mammal under conditions where the number of cancer cells present within the mammal is reduced. The sample can include at least one cancer cell. The sample can be a tissue sample. The mammal can be a human. The cancer immunotherapy can be alemtuzumab, atezolizumab, avelumab, ipilimumab, ofatumumab, nivolumab, pembrolizumab, rituximab, or durvalumab. The mammal also can be administered an additional cancer treatment. The additional cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, identifying a sample from the mammal as having a molecular smoking signature; and administering a cancer immunotherapy to the mammal under conditions wherein the number of cancer cells present within the mammal is reduced. The sample can include at least one cancer cell. The sample can be a tissue sample. The mammal can be a human. The cancer immunotherapy can be alemtuzumab, atezolizumab, avelumab, ipilimumab, ofatumumab, nivolumab, pembrolizumab, rituximab, or durvalumab. The mammal also can be administered an additional cancer treatment. The additional cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, administering a cancer immunotherapy to a mammal identified as having at least one cancer cell having a mutation in an ARID1A nucleic acid sequence. The mammal can be a human. The cancer immunotherapy can be alemtuzumab, atezolizumab, avelumab, ipilimumab, ofatumumab, nivolumab, pembrolizumab, rituximab, or durvalumab. The mammal also can be administered an additional cancer treatment. The additional cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, administering a cancer immunotherapy to a mammal identified as having at least one cancer cell with a molecular smoking signature. The mammal can be a human. The cancer immunotherapy can be alemtuzumab, atezolizumab, avelumab, ipilimumab, ofatumumab, nivolumab, pembrolizumab, rituximab, or durvalumab. The mammal also can be administered an additional cancer treatment. The additional cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, identifying a sample from the mammal as an activating mutation in EGFR nucleic acid, an activating mutation in ERBB2 nucleic acid, an activating mutation in MET nucleic acid, an activating mutation in FGFR1 nucleic acid, or an activating mutation in IGF1R nucleic acid; and administering a cancer treatment to the mammal under conditions where the number of cancer cells present within the mammal is reduced, and where the cancer treatment is not a cancer immunotherapy. The sample can include at least one cancer cell. The sample can be a tissue sample. The mammal can be a human. The cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, identifying a sample from the mammal as having germline homozygosity or a loss of at least one HLA class I locus; and administering a cancer treatment to the mammal under conditions where the number of cancer cells present within the mammal is reduced, and where the cancer treatment is not a cancer immunotherapy. The sample can include at least one cancer cell. The sample can be a tissue sample. The mammal can be a human. The cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, identifying a sample from the mammal as having a mutation in a KEAP1 nucleic acid sequence; and administering a cancer treatment to the mammal, and where the cancer treatment is not a cancer immunotherapy. The sample can include at least one cancer cell. The sample can be a tissue sample. The mammal can be a human. The cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, administering a cancer treatment to a mammal identified as having at least one cancer cell having an activating mutation in EGFR nucleic acid, an activating mutation in ERBB2 nucleic acid, an activating mutation in MET nucleic acid, an activating mutation in FGFR1 nucleic acid, or an activating mutation in IGF1R nucleic acid, where the cancer treatment is not a cancer immunotherapy. The mammal can be a human. The cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, administering a cancer treatment to a mammal identified as having germline homozygosity or a loss of at least one HLA class I locus, where the cancer treatment is not a cancer immunotherapy. The mammal can be a human. The cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for treating mammals having cancer where the methods can include, or consist essentially of, administering a cancer treatment to a mammal identified as having a mutation in a KEAP1 nucleic acid sequence, where the cancer treatment is not a cancer immunotherapy. The mammal can be a human. The cancer treatment can be surgery, radiation therapy, administration of a chemotherapy, administration of a hormone therapy, administration of a targeted therapy, or administration of a cytotoxic therapy. The cancer can be a lung cancer (e.g., a non-small cell lung cancer, a lung squamous cell carcinoma, or a lung adenocarcinoma).

In another aspect, this document features methods for identifying a mammal as having a cancer that is likely to respond to an immunotherapy. The methods can include, or consist essentially of, determining a cTMB of the cancer, determining a mutational signature of the cancer, and identifying the cancer as not being likely to respond to an immunotherapy when the mutational signature of the cancer includes i) an activating mutation in a nucleic acid encoding a receptor tyrosine kinase (RTK) polypeptide; and ii) germline homozygosity or a loss of at least one HLA class I locus. The nucleic acid encoding the RTK polypeptide is a EGFR, ERBB2, MET, FGFR1, or IGF1R nucleic acid. Determining the cTMB of the cancer can include determining an observed TMB (obsTMB) of a sample including at least one cancer cell from the cancer, determining a tumor purity (a) of the sample, and adjusting the observed TMB based on the tumor purity using a correction factor (r) as set forth in Table 4 to determine the cTMB. The method of cTMB can be determined using the equation cTMB=r(α)*obsTMB. The cancer can be a lung cancer (e.g., a non-small cell lung cancer, a lung squamous cell carcinoma, or a lung adenocarcinoma).

In another aspect, this document features methods for identifying a mammal as having a cancer that is likely to respond to an immunotherapy. The methods can include, or consist essentially of, determining a cTMB of the cancer, determining a mutational signature of the cancer, and identifying the cancer as being likely to respond to the immunotherapy when the mutational signature of the cancer includes i) mutation in an ARID1A nucleic acid sequence or a molecular smoking signature; and ii) germline heterozygosity at least one HLA class I locus. The molecular smoking signature can include cytosine (C) to adenosine (A) transversions (C>A transversions). Determining the cTMB of the cancer can include determining an observed TMB (obsTMB) of a sample including at least one cancer cell from the cancer, determining a tumor purity (a) of the sample, and adjusting the observed TMB based on the tumor purity using a correction factor (r) as set forth in Table 4 to determine the cTMB. The method of cTMB can be determined using the equation cTMB=r(α)*obsTMB. The cancer can be a lung cancer (e.g., a non-small cell lung cancer, a lung squamous cell carcinoma, or a lung adenocarcinoma).

In another aspect, this document features methods for determining a cTMB. The methods can include, or consist essentially of, determining an obsTMB of a sample including at least one cancer cell; determining a tumor purity (a) of the sample; and adjusting the observed TMB based on the tumor purity using a correction factor (r) as set forth in Table 4 to determine the cTMB. The cTMB can be determined using the equation cTMB=r(α)*obsTMB.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 (includes FIGS. 1A-10. Evaluation of the impact of tumor purity and clonal heterogeneity on TMB estimates. Mutation burden was estimated for 2 in silico tumor samples, a high mutator with high intratumoral clonal heterogeneity (A, B) and a low mutator with low intratumoral heterogeneity (C, D), across a wide range of tumor purity values (0.2-1.0, shown in the header of each graph). Mutant allele frequency-MAF distributions are shown for a simulated tumor with true TMB of 265 and 4 mutation clusters (C1-C₄); C1 with 100 clonal mutations (cellular fraction; CF=1.00), C2 with 50 mutations at CF=0.70, C3 with 40 mutations at CF=0.40, and C4 with 75 mutations at CF=0.20 at different tumor purity levels (A). The dotted line indicates a MAF of 10%, which is the threshold used for somatic mutation calling. Power of detection of different subclones decreased with decreasing tumor purity resulting in a decline in TMB estimation accuracy (B). The blue line and ribbon mark the median and range of estimated TMB across 10 replicates, while the red dotted line indicates the true TMB of the tumor. MAF distributions are shown for a simulated homogeneous tumor with true TMB of 150 and two mutation clusters (C1-C2); C1 with 100 clonal mutations (CF=1.00), and C2 with 50 mutations at CF=0.50 at different tumor purity levels (C). Estimated TMB for the tumor in (C) at each purity level shows that TMB estimates remain accurate for lower tumor purity tiers compared to the more heterogeneous tumor in (A). As tumor purity decreases below 40%, TMB estimates converge. Panel headers indicate tumor purity and estimated TMB in (A) and (C) and cellular fraction refers to the fraction of cancer cells harboring a mutation. Analysis of paired tumor-normal whole exome sequencing data from TCGA samples with tumor purity less than 50% revealed a positive correlation between TMB and tumor purity in head and neck cancer (R=0.33, p=0.05; E), renal clear cell carcinoma (R=0.48, p=0.0003; F), lung adenocarcinoma (R=0.18, p=0.09; G) and lung squamous cell carcinoma (R=0.39, p=0.002; H). A linear model was fitted to the mutation sequence data for each tumor type. TMB scores derived from targeted sequencing highly correlated with tumor purity assessments (Spearman rho=0.29, p<0.0001; I). HNSCC; head and neck squamous cell carcinoma, KIRC; kidney renal clear cell carcinoma, LUAD; lung adenocarcinoma, LUSC; lung squamous cell carcinoma, NSCLC; non-small cell lung cancer.

FIG. 2. Tumor purity correlates with TMB estimates from higher sequencing depth targeted next-generation sequencing. TMB scores derived from targeted sequencing and tumor purity assessments were retrieved from a published cohort of 1,661 tumors treated with immune checkpoint blockade (Samstein et al., Nature genetics, doi:10.1038/s41588-018-0312-8 (2019)) and non-parametric correlations were evaluated. A significant correlation between TMB and tumor purity was identified for NSCLC (Spearman rho=0.29, p<0.0001), bladder cancer (rho=0.18, p=0.03), esophagogastric cancer (rho=0.19, p=0.05) and head and neck cancer (rho=0.18, p=0.07).

FIG. 3 (includes FIGS. 3A-3F). Correlation of tumor purity with tumor mutational burden and clinical response in 957 TCGA NSCLC samples and the two immunotherapy NSCLC cohorts. TCGA lung adenocarcinomas-LUAD (A) and lung squamous cell carcinomas-LUSC (B) with a higher degree of normal contamination had a significantly lower TMB compared to tumors with a tumor purity >50% (Mann-Whitney p=0.06 and p<0.001 for LUAD and LUSC respectively). In the two immunotherapy treated NSCLC cohorts, the correlation between TMB and tumor purity was particularly pronounced for tumor purity less than 30% (p=0.008 and P=0.08 for overall comparisons of TMB across tumor purity tiers for cohort 1 and cohort 2 respectively (C-D). Tumor purity was associated with clinical benefit from ICB when mutation-based purity was used, which is most likely attributed to the contribution of TMB in the mutation-based purity calculation; however no difference in tumor purity was found between responding and non-responding tumors when copy-number based tumor purity and adjusted tumor purity was used in cohort 1 (E) and cohort 2 (F).

FIG. 4 (includes FIGS. 4A-4F). Impact of corrected TMB and single genomic biomarkers on overall survival. Through simulation analyses correction factors were developed for different tumor purity values (A) and we determined corrected TMB values for the tumors our cohort. Patients with higher observed TMB (using the second tertile as a threshold) had marginally longer overall survival (log rank p=0.048; B); the association of TMB with overall survival became more significant after TMB was corrected for tumor purity (log rank p=0.014; C). Patients with a molecular smoking mutational signature derived durable benefit from immune checkpoint blockade (log rank p=0.031; D). Activating RTK mutations identified a group of patients with dismal prognosis in both cohort 1 (log rank p=0.005) and cohort 2 (log rank p=0.009). cTMB; corrected TMB, RTK; receptor tyrosine kinase.

FIG. 5. Genomic drivers associated with response to immune checkpoint blockade. Responding tumors had a higher total and clonal observed TMB compared to non-responding tumors (p=0.002, FDRadjusted p=0.012 and p<0.001, FDR-adjusted p=0.005 respectively), however there was considerable overlap in the TMB range between responding and non-responding tumors. There were no differences in tumor purity and tumor aneuploidy between responding and non-responding tumors. Overall, a higher number of single base substation and indels were found in responding tumors, which was largely driven by their higher TMB. An enrichment in the C>A transversion-rich molecular smoking signature was found in patients with durable clinical benefit (p=0.003, FDR-adjusted p=0.027). Activating mutations in RTK genes (EGFR and ERBB2 point mutations and amplifications, MET amplification, FGFR1 amplification and IGF1R amplification) were found to cluster in patients that did not derive durable clinical benefit from immune checkpoint blockade (p<0.001, FDR-adjusted p=0.002) independent of TMB (TMB-adjusted p=0.04). Recurrent genomic alterations in ARID1A, including truncating mutations in the setting of LOH of the wild-type allele, were predominantly found in patients with durable clinical benefit (p=0.005, FDRadjusted p=0.024, TMB-adjusted p=0.062). A trend towards enrichment in KEAP1 mutations, especially in the context of biallellic inactivation was found in patients without durable clinical benefit (TMB-adjusted p=0.074). We did not detect any loss-of-function mutations in JAK1 or JAK2 or an enrichment of cooccurring KRAS and inactivating STK11 mutations in non-responding tumors. A homozygous deletion in PTEN was found in a patient with a short-lived response to immune checkpoint blockade and MDM2/MDM4 amplifications were identified in 3 non responders. CNV; copy number variation.

FIG. 6. Distribution of observed (black circles) and corrected TMB for patients in cohort 1 are shown for each tumor purity tier. Corrected TMB values are denoted by purple circles for tumor purity 0.1-0.25 and green circles for tumor purity >0.25, error bars represent 95% confidence intervals. cTMB values are capped at 1000. After correction for tumor purity cases 5 patients were reclassified from low mutators to high mutators. DCB; durable clinical benefit, NDB; non-durable clinical benefit, NA; radiographic response non evaluable.

FIG. 7 (includes FIGS. 7A-7B). In silico dilution experiment of single base substitutions to evaluate the power to accurately determine contribution of a dominant mutation signature. Mutation signature analyses were performed on whole exome data from 985 NSCLC tumors (508 lung adenocarcinomas and 477 squamous cell carcinomas) obtained through TCGA. Seventy-six NSCLC tumors (64 lung adenocarcinomas and 12 squamous cell carcinomas) had a tumor mutation load >250 and a molecular smoking signature >75% and were further selected for an in silico dilution series. Mutation counts were diluted from maximum count to a minimum of 5 using random resampling, to evaluate consistency and divergence in the predicted presence of a smoking signature (A). On average, 20 mutations were sufficient to predict the presence of a smoking signature at a 50% level. Mutational load below 20 mutations lead to a 30% difference from the original contribution of the C>A transversion rich signature value and therefore represents a threshold beyond which, there is a significant deviation from accurately determining a dominant mutation signature (B).

FIG. 8 (includes FIGS. 8A-8B). Genomic drivers associated with response to immune checkpoint blockade in cohort 2 and impact of RTK mutations on outcome in cohort 3. Responding tumors had a higher total and clonal TMB compared to non-responding tumors (Mann Whitney p=0.006 and p=0.004 respectively; A). Progression-free survival, histology and tumor purity are shown as separate panels. Patients with a clinical response had a higher contribution of the molecular smoking signature (Mann Whitney p=0.054). There were no differences in tumor aneuploidy between responders and non-responders (Mann Whitney p=0.72). A significant enrichment in RTK activating mutations, including point mutations and amplifications in EGFR, amplifications in ERBB2 and MET exon 14 skipping, was found in non-responding tumors (chi square p=0.056). A third cohort of NSCLC patients treated with ICB was obtained from CBioportal; for this cohort sequence and copy number alterations alongside with outcome information was publicly available. Patients with activating RTK mutations in EGFR, ERBB2, MET, FGFR1 and IGF1R had a significantly shorter progression-free survival (log rank p=0.035; B). CNV; copy number variation.

FIG. 9 (includes FIGS. 9A-9C). Co-deletion of IFN-related genes in tumors with CDKN2A homozygous deletions. Given that deletions in IFN-γ genes have been described as a potential mechanism of intrinsic resistance to immunotherapy, we investigated whether there is an enrichment in IFN-γ related gene copy number variation in non-responding tumors. A cluster of IFN-γ related genes (IFNE, IFNA1, IFNA2, IFNA4, IFNA5, IFNA6, IFNA8, IFNA14, IFNA21, IFNW1 and IFNB1) is located on chromosome 9 (p21.3), in close proximity to the CDKN2A locus (A). The locus that contains both the IFN-γ related genes and CDKN2A was frequently found to be deleted; an example of such homozygous deletion is shown for case CGLU262 (B). The vertical axes denote the relative copy ratio (log 2 scale), and the integer copy number levels assigned to genomic bins (circles) and segments. Purple and green boxes mark the coordinates of IFN gene cluster and CDKN2A, respectively. The frequency of homozygous deletions in IFNE, IFNA1, IFNA2, IFNA4, IFNA5, IFNA6, IFNA8, IFNA14, IFNA21, IFNW1 and IFNB1 was similar in responding and non-responding tumors and more importantly, these deletions co-occurred with CDKN2A loss in 86% of the cases, whereas CDKN2A deletions also occurred independently (C). Given that, CDKN2A and the group of IFN-γ pathway genes lie on chromosome 9p within a span of 917 Kb, IFN-γ deletions may be passengers in the setting of a driver CDKN2A deletion. CNA: copy number aberration.

FIG. 10. Pathway enrichment analysis for DNA damage repair genes and the wnt pathway in cohort 1. We investigated co-occurrence of mutations in DNA damage repair genes involved in base excision repair (DDR-BER), DNA damage sensoring, the Fanconi anemia pathway (FA), homologous recombination (DDR-HR), mismatch repair (DDR-MMR), nucleotide excision repair (DDR-NER), non-homologous end joining (DDR-NHEJ) and translesion DNA synthesis (DDR-TLS). Mutations were characterized by consequence (missense, frameshift, nonsense, splice site, in-frame) and recurrence (hotspots) and loss of the wild type allele was considered in case of truncating mutations (biallellic inactivation). A similar analysis was performed for genes involved in the wnt pathway. A high TMB tumor with biallellic inactivation of MLH1 and a tumor with a gain-of-function beta-catenin hotspot mutation were identified among responders and non-responders respectively, with no additional significant differences in genomic alterations in the DDR and WNT pathways between responders and non-responders.

FIG. 11. Large-scale copy number analyses for NSCLC tumors in cohort 1. A genome-wide analysis of copy number profiles revealed genomic regions with copy number gains and losses and was used to determine the extent of tumor aneuploidy. The relative copy ratio (Log R) values quantifying the abundance of each genomic region compared to the genome average (ploidy) are shown after correction for tumor purity in responding and non-responding tumors. Red and blue shades indicate copy gains and losses, respectively, whereas white marks copy neutral regions. There was no significant difference in the degree of aneuploidy assessed by the fraction of genome with allelic imbalance between the two groups (Mann Whitney p=0.367, FDR-corrected p=0.65).

FIG. 12. MANA characteristics for NSCLC tumors in cohort 1. The distributions of total MANA load and fit MANA load are shown in the top panel. Responding tumors harbored a higher load of fit MANAs (determined as neopeptides with a predicted MHC affinity <50 nM for which the wild type peptides has a predicted MHC affinity of >1000 nM) compared to non-responding tumors (Mann-Whitney p=0.01). MANAs derived from frameshift mutations were compared between responders and nonresponders after filtering out those most likely to undergo nonsense mediated decay; a higher MANA load stemming from frameshifts was found in responders (p=0.08). The cumulative length of frameshifts until reaching a stop codon was assessed after correcting for nonsense mediated decay and TMB; no differences were found between responding and non-responding tumors. Neopeptides RLDGHTSL, FYSRAPEL and HRHPPVAL stemming from frameshift mutations in SH2D7, ADAMTS12 and KLHL42, found in 3 responding tumors, had a high homology to Mycobacterium leprae, Mycobacterium tuberculosis and HHV5 antigens respectively. FS; frameshift, NMD; nonsense mediated decay, Hom; homologous.

FIG. 13. Distribution of hotspot mutations and associated potentially immunogenic MANAs in NSCLC tumors with differential responses to immune checkpoint blockade. The number of mutations with at least one fit MANA (determined as neopeptides with a predicted MHC affinity <50 nM for which the wild type peptides has a predicted MHC affinity of >1000 nM) in each tumor, divided by clonality and hotspot status is shown in the top distribution graph. Clinical response and overall survival are shown in the middle panel. Clonal hotspot frameshifts and in-frame insertions and deletions in ANTRX2, TP53, EGFR, ASXL1, NOTCH2, ZFP36L2, FAM171B, SLC35F5, CD93 and SLAMF1 generated fit MANAs shown in the lower panel. There was no difference in the number of clonal fit MANAs between responding and non-responding tumors. NDB: No durable benefit, DCB: durable clinical benefit.

FIG. 14 (includes FIGS. 14A-14D). HLA class I genetic variation and association with response to immune checkpoint blockade. The number of HLA class I germline alleles is shown in (A), with no differences in the degree in homozygosity found between responders and non-responders. HLA class I somatic mutations were infrequent. HLA class I germline zygosity and somatic HLA class I LOH events were combined to calculate the unique number of HLA class I alleles on cancer cells. We identified one tumor with homozygous loss of HLA-B in patient CGLU310 who achieved durable clinical benefit from anti-PD1 therapy without evidence of disease progression 14 months after treatment initiation, suggesting that response may be attributed to NK-cell mediated cell lysis in the setting of HLA class I homozygous deletion. There was no evidence of biallellic inactivation of β2-microglobulin in cohort 1. Tumors with reduced antigen presentation potential (<5 unique tumor HLA class I alleles) were linked to worse outcome (log rank p=0.08; B), this observation was more prominent when the number of HLA class I alleles in the tumor was combined with TMB. Patients with low TMB and reduced antigen presentation potential had a significantly shorter overall survival (log rank p=0.01; C). Tumors with lower antigen presentation capacity showed a significantly lower level of CD8+ T cell density (Mann Whitney p=0.005; D).

FIG. 15. Frequency of loss of heterozygosity at a chromosomal arm level in 11 tumor types. We investigated whether there is an enrichment for chromosome 6p-contains the HLA class I loci-LOH events in NSCLC compared to the background arm-level allelic imbalance of the same tumor type and across tumor types. Chromosome 6p losses were not more frequent compared to other chromosomal arm level deletions (on the contrary the degree of chromosome 6p LOH was lower compared to other chromosomal arms deletions in lung tumors, p=0.037). In contrast, when chromosome 6p LOH events were compared between lung tumors and 9 tumor types (BLCA, BRCA, COAD, GBM, HNSC, KIRC, OV, READ and SKCM, n=3,674), we found that LOH events involving chromosome 6p that contains the HLA class I loci are more frequent in lung cancer (17.3% vs. 8.2%, p<0.001), without any evidence for positive selection of these events in advanced stage disease. BLCA; bladder urothelial carcinoma, BRCA; breast invasive carcinoma, COAD; colon adenocarcinoma, GBM; glioblastoma, HNSC; head and neck squamous cell carcinoma, KIRC; kidney clear cell carcinoma, LUAD; lung adenocarcinoma, LUSC; lung squamous cell carcinoma, OV; ovarian cancer, READ; SKCM; skin cutaneous melanoma.

FIG. 16. Correlation between tumor mutation burden and degree of germline HLA homozygosity and somatic HLA LOH by stage. Kruskal-Wallis one-way analysis of variance was applied to assess the correlation of germline homozygosity in HLA class I genes with tumor mutation burden in 6 tumor types (BLCA, BRCA, COAD, HNSCC, KIRC, LUAD and LUSC, n=3,601). Germline HLA zygosity was not correlated with TMB for the vast majority of tumors examined with the exception of bladder cancer (p=0.02). Germline and tumor HLA class I status was combined to determine the number of unique HLA class I alleles in each tumor. The number of unique HLA class I alleles appeared to correlate with TMB such that tumors with a higher number of unique HLA class I alleles in the tumor, that would therefore have a more intact antigen presentation capacity, harbored a lower non-synonymous mutation load for BLCA (p=0.02) and HNSCC (p=0.07). When tumors heterozygous for all three HLA class I loci (6 HLA class I alleles) where compared to tumors that were homozygous in all three HLA class I loci (3 HLA class I alleles), a higher TMB was noted in the tumors with the more intact antigen presentation capacity (Wilcoxon p=0.05 for BLCA, p=0.09 for BRCA, p=0.01 for HNSCC, p=0.01 for LUAD).

FIG. 17 (includes FIGS. 17A-17I). HLA class I distribution by supertype and association with TMB and outcome. Individual HLA-I alleles were classified into discrete supertypes, based upon similar peptide-anchorbinding specificities. HLA-A supertype distribution is shown in (A) for cases in cohort 1. TMB did not differ among different HLA-A supertypes (B) and there was no association with overall survival (C). The same observations held true for HLA-B supertype analyses (D-F). Germline HLA class I variation was not associated with outcome (G), however there was a trend towards longer overall survival for TMB high tumors with maximal germline HLA class I heterozygosity (H). Cases with maximal germline HLA class I heterozygosity were found to have a less clonal TCR repertoire (I).

FIG. 18 (includes FIGS. 18A-18C). Multivariable model for prediction of outcome to immune checkpoint blockade. cTMB, RTK mutations, molecular smoking signature and HLA germline variation were combined in a multivariable Cox proportional hazards regression model and a risk score was calculated for each case based on the weighted contribution of each parameter (A). Patients with a higher risk score had a significantly shorter overall survival in cohort 1 (13 vs. 38 months, HR=3.29, 95% CI: 1.77-6.14, log rank p=0.0001; B) and progression-free survival in cohort 2 (3 vs. 8 months, HR=2.73, 95% CI 1.15-6.45, log rank p=0.017; C).

### DETAILED DESCRIPTION

This document provides methods and materials for assessing and/or treating a mammal having a cancer. For example, this document provides methods and materials for identifying a mammal having a cancer as being likely to be responsive to a particular cancer treatment (e.g., by detecting a cTMB of one or more cells such as cancer cells from the mammal), and, optionally, treating the mammal. In some cases, the methods and materials described herein can be used to predict response to a particular cancer treatment (e.g., a cancer immunotherapy). For example, a sample obtained from a mammal (e.g., a human) having a cancer can be assessed to determine if the mammal is likely to be responsive to a particular cancer treatment (e.g., a cancer immunotherapy) based, at least in part, on the cTMB of the sample and/or on a multivariable model including the cTMB, the presence of one or more mutations in one or more nucleic acid sequences encoding a RTK polypeptide, the ability to present one or more antigens (e.g., HLA germline variation), and/or the presence of a smoking-related mutational signature in the sample.

In some cases, the methods and materials described herein can be used to treat a mammal having a cancer. For example, a mammal having a cancer identified as being likely to be responsive to a particular cancer treatment based, at least in part, on the cTMB of the sample from the mammal, can be treated with that particular cancer treatment as described herein. In some cases, a mammal having a cancer identified as being likely to be responsive to a cancer immunotherapy based, at least in part, on the cTMB of the sample from the mammal, can be treated with a cancer immunotherapy as described herein. In some cases, the methods and materials described herein can be used to improve progression-free survival. In some cases, the methods and materials described herein can be used to improve disease-free (e.g., relapse-free) survival. In some cases, the methods and materials described herein can be used to improve overall survival.

When treating a mammal having a cancer as described herein, the treatment can be effective to treat the cancer (e.g., to reduce one or more symptoms of the cancer). In some cases, the number of cancer cells present within a mammal can be reduced using the materials and methods described herein. In some cases, the size (e.g., volume) of one or more tumors present within a mammal can be reduced using the materials and methods described herein. In some cases, the size (e.g., volume) of one or more tumors present within a mammal does not increase.

When treating a mammal having a cancer as described herein, the treatment can be effective to treat the cancer (e.g., to reduce one or more symptoms of the cancer) with reduced or eliminated complications associate with that treatment. For example, when the treatment is a cancer immunotherapy, the cancer immunotherapy can be administered to a mammal having cancer, and identified as being likely to be responsive to a cancer immunotherapy (e.g., by detecting a cTMB of one or more cells such as cancer cells from the mammal), with reduced or eliminated toxicity from the cancer immunotherapy. For example, when the treatment is a cancer immunotherapy, the cancer immunotherapy can be administered to a mammal having cancer, and identified as being likely to be responsive to a cancer immunotherapy (e.g., by detecting a cTMB of one or more cancer cells from the mammal), with reduced or eliminated infection from the cancer immunotherapy.

Any type of mammal having a cancer can be assessed and/or treated as described herein. Examples of mammals that can be assessed and/or treated as described herein include, without limitation, primates (e.g., humans and monkeys), dogs, cats, horses, cows, pigs, sheep, rabbits, mice, and rats. In some cases, a human having a cancer can be assessed to determine if the human is likely to be responsive to a particular cancer treatment based, at least in part, on the cTMB of the sample and, optionally, can be treated with that particular cancer treatment as described herein.

A mammal having any type of cancer (e.g., a cancer including one or more cancer cells) can be assessed and/or treated as described herein. In some cases, a cancer can include one or more tumors (e.g., one or more solid tumors). In some cases, a cancer can be a blood cancer. Examples of cancers that can be assessed and/or treated as described herein include, without limitation, lung cancers (e.g., non-small cell lung cancers such as lung squamous cell carcinoma and lung adenocarcinoma), breast cancers (e.g., breast carcinomas such as breast invasive carcinoma), prostate cancers, ovarian cancers, gastric cancers (e.g., gastroesophageal cancers), endometrial cancers, bladder cancers (e.g., bladder carcinomas such as bladder urothelial carcinoma), colon cancers (e.g., colon adenocarcinomas), brain cancers (e.g., glioblastomas), head and neck cancers (e.g., head and neck squamous cell carcinomas), kidney cancers (e.g., kidney clear cell carcinomas), and skin cancers (e.g., melanomas such as skin cutaneous melanoma).

In some cases, a mammal can be identified as having a cancer. Any appropriate method can be used to identify a mammal as having a cancer. For example, imaging techniques and biopsy techniques can be used to identify mammals (e.g., humans) as having cancer.

A mammal having a cancer can be assessed as described herein to determine whether or not it is likely to respond to a particular cancer treatment (e.g., a cancer immunotherapy). For example, a sample (e.g., a sample including one or more cancer cells) obtained from the mammal can be assessed for the cTMB as described herein, and the cTMB of one or more cancer cells from that mammal can be used to determine whether or not that mammal is likely to respond to a particular cancer treatment.

Any appropriate sample from a mammal (e.g., a human) having a cancer can be assessed as described herein. In some cases, a sample can be a biological sample. For example, a sample can be a tumor sample. In some cases, a tumor sample can contain at least a portion of a tumor. In some cases, a sample can contain one or more cancer cells. Examples of samples that can be assessed as described herein include, without limitation, tissue samples (e.g., colon tissue samples, rectum tissue samples, and skin tissue samples), stool samples, cellular samples (e.g., buccal samples), and fluid samples (e.g., blood, serum, plasma, urine, and saliva). A sample can be a fresh sample or a fixed sample. In some cases, a sample can be an embedded (e.g., paraffin embedded or OCT embedded) sample. In some cases, a sample can be processed (e.g., processed to isolate and/or extract one or more biological molecules such as nucleic acids and polypeptides).

In some cases, a cTMB of one or more cells (e.g., one or more cancer cells) from a mammal can be used to identify that mammal as being likely to be responsive to a cancer immunotherapy. As used herein a cTMB is a TMB that is adjusted for tumor purity. In some cases, a cTMB can include an increased number of mutations (e.g., as compared to a TMB that has not been corrected as described herein and/or as compared to a sample having low tumor purity). For example, a higher cTMB score can be used to identify that mammal as being likely to be responsive to a cancer immunotherapy. In some cases, a higher cTMB score can be a score that is within the top 20-30% of cTMB scores in a given cohort. For example, mammals having a cTMB score that is within the top 20-30% of cTMB scores in a given cohort can be identified as likely to be responsive to a cancer immunotherapy.

Any appropriate method can be used to obtain a cTMB. For example, a TMB (e.g., an observed TMB (obsTMB)) of a sample (e.g., a sample obtained from a mammal) can be adjusted, based at least in part on the tumor purity of the sample, to obtain a cTMB. A TMB can be determined using any appropriate method. For example, whole exome sequencing and targeted next-generation sequencing can be used to determine a TMB. As used herein, “tumor purity” refers to the percentage of cells in a sample (e.g., a sample obtained from a mammal) that are cancer cells. The tumor purity of a sample can be obtained using any appropriate method. For example, whole exome sequencing, and/or targeted next-generation sequencing can be used to determine the tumor purity of a sample. In some cases, a cTMB can be corrected for tumor purity using correction factors for particular tumor purity values. Correction factors for particular tumor purity values can be as described in Table 4. For example, a cTMB can be determined using the equation

cTMB=r(α)*obsTMB

where r is the correction factor and a is the tumor purity. In some cases, a cTMB can be corrected for tumor purity as described in Example 1.

A cTMB can include any number of mutations. In some cases, the number of mutations found in a cell can be referred to as the mutational load of the cell. In some cases, a mutational signature can include from about 1 mutation to about several thousands of mutations. For example, a cTMB can include from about 5 mutations to about 100 mutations. In some cases, a cTMB can include at least about 20 mutations.

A cTMB can include any appropriate mutational signature (e.g., can include any mutations found in a cell, such as a cancer cell, from a mammal). As used herein a “mutational signature” is a characteristic combination of mutations. A mutational signature can include any appropriate types of mutations. In some cases, a mutation can be a somatic mutation. In some cases, a mutation can be an activating mutation. In some cases, a mutation can be a loss of function mutation (e.g., an inactivating mutation). Examples of types of mutations that can be included in a mutational signature can include, without limitation, substitutions such as transversions (e.g., point mutations such as C>A transversions), insertions (e.g., in-frame insertions or frameshift insertions), deletions (e.g., gene deletions such as in-frame deletions or frameshift deletions and/or chromosomal deletions), insertion/deletions (indels; e.g., in-frame indels or frameshift indels), and truncating mutations. A mutation that can be included in a mutational signature can be any appropriate location within the genome of a cell (e.g., a cancer cell). In some cases, a mutation included in a mutational signature can be in a coding sequence (e.g., a nucleotide sequence that encodes a polypeptide). In some cases, a mutation included in a mutational signature can be in non-coding sequence. In some cases, a mutation included in a mutational signature can be in a splice site. In some cases, a mutation included in a mutational signature can be in regulatory region (e.g., a nucleotide sequence that controls expression of a polypeptide such as a promoter sequence or an enhancer sequence). When a mutation that can be included in a mutational signature is in a coding sequence (or a regulatory region that control expression of that coding sequence), the mutation can be in any appropriate coding sequence. In some cases, a mutation that can be included in a mutational signature can be in a coding sequence (or a regulatory region that control expression of that coding sequence) that encodes a RTK polypeptide. In some cases, a mutation that can be included in a mutational signature can be in a coding sequence (or a regulatory region that control expression of that coding sequence) that encodes a polypeptide involved in DNA damage repair (DDR). In some cases, a mutation that can be included in a mutational signature can be in a coding sequence (or a regulatory region that control expression of that coding sequence) that encodes a polypeptide involved in the WNT-β-catenin pathway. In some cases, a mutation that can be included in a mutational signature can be in a coding sequence (or a regulatory region that control expression of that coding sequence) that encodes a polypeptide involved in an immune-related pathway (e.g., the IFNγ pathway). In some cases, a mutation that can be included in a mutational signature can be in a coding sequence (or a regulatory region that can control expression of that coding sequence) that encodes a polypeptide involved in the PI3K-AKT-mTOR pathway. Examples of nucleic acid (coding sequences or regulatory regions that control expression of that coding sequence) that can include one or more mutations in a mutational signature can include, without limitation, EGFR, ERBB2, MET, FGFR1, IGF1R, ARID1A, KEAP1, JAK1, JAK2, KRAS, STK11, PTEN, MDM2, and MDM4 nucleic acid. In some cases, a mutation that can be included in a mutational signature and can be used to identify that mammal as being likely to be responsive to a cancer immunotherapy can be as described in Example 1. In some cases, a mutation that can be included in a mutational signature and can be used to identify that mammal as being likely to be responsive to a cancer immunotherapy can be as described in one or more examples, Tables and/or Figures herein.

Any appropriate method can be used to detect one or more mutations in the genome of a cell (e.g., a cancer cell). In some cases, one or more mutations can be detected in the genome of a cell using sequencing techniques (e.g., PCR-based sequencing such as Next-Generation PCR-based sequencing and Sanger sequencing), DNA hybridization techniques, and/or restriction enzyme digestion methods.

In some cases, the presence or absence of one or more mutations in one or more nucleic acid sequences encoding a RTK polypeptide (e.g., a RTK nucleic acid) can be used to identify that mammal as being likely to be responsive to a cancer immunotherapy. For example, detecting one or more mutations in one or more nucleic acid sequences encoding a RTK polypeptide in the genome of one or more cells (e.g., one or more cancer cells) from a mammal can be used to identify that mammal as being likely to be responsive to a cancer immunotherapy. A mutation included in nucleic acid sequence encoding a RTK polypeptide can be a somatic mutation or a germline mutation. A mutation in nucleic acid sequence encoding a RTK polypeptide can be an activating mutation or a loss of function mutation (e.g., an inactivating mutation). Examples of types of mutations that can be present in nucleic acid sequence encoding a RTK polypeptide can include, without limitation, substitutions such as transversions (e.g., C>A transversions), insertions (e.g., in-frame insertions or frameshift insertions), deletions (e.g., in-frame deletions or frameshift deletions), insertion/deletions (indels; e.g., in-frame indels or frameshift indels), amplifications, and truncating mutations. Examples of nucleic acid sequences that can encoding a RTK polypeptide can include, without limitation, EGFR, ERBB2, MET, FGFR1, and IGF1R nucleic acids. For example, one or more point mutations in EGFR nucleic acid (e.g., point mutations in EGFR exon 21 such as L858R), one or more point mutations in ERBB2 nucleic acid (e.g., point mutations in ERBB2 exon 19 such as E770 A771insAYVM), one or more point mutations in MET nucleic acid, one or more point mutations in FGFR1 nucleic acid, and/or one or more point mutations in IGF1R nucleic acid; an amplification of FGFR1 nucleic acid and/or an amplification of IGF1R nucleic acid; both one or more point mutations in and an amplification of EGFR nucleic acid, both one or more point mutations in and an amplification of ERBB2 nucleic acid, and/or both one or more point mutations in and an amplification of MET nucleic acid; an in-frame deletion in EGFR nucleic acid (e.g., in-frame deletions in EGFR exon 19 such as 745KELREA>T, E746 A750del, and L747_T751del); an in-frame insertion in EGFR nucleic acid (e.g., frame insertions in EGFR exon 20 such as N771 H773dup); and/or an in-frame insertion in ERBB2 nucleic acid (e.g., frame insertions in ERBB2 exon 20 such as 776G>VC) can be used to identify that mammal as being likely to be responsive to a cancer immunotherapy. In some cases, a mutation in nucleic acid sequence encoding a RTK polypeptide that can be used to identify that mammal as being likely to be responsive to a cancer immunotherapy can be as described in Example 1. In some cases, a mutation in nucleic acid sequence encoding a RTK polypeptide that can be used to identify that mammal as being likely to be responsive to a cancer immunotherapy can be as described in Tables 3, 5, 6 and/or 7.

Any appropriate method can be used to detect one or more mutations in the genome of a cell (e.g., a cancer cell). In some cases, one or more mutations can be detected in the genome of a cell using sequencing techniques (e.g., PCR-based sequencing such as Next-Generation PCR-based sequencing and Sanger sequencing), DNA hybridization techniques, and/or restriction enzyme digestion methods.

In some cases, the ability of one or more cells (e.g., one or more cancer cells) from a mammal to present one or more antigens (e.g., one or more tumor antigens such as MANAs) can be used to identify that mammal as being likely to be responsive to a cancer immunotherapy. For example, detecting one or more mutations that can reduce the antigen presentation potential of one or more cells (e.g., one or more cancer cells) from a mammal can be used to identify that mammal as being likely to be responsive to a cancer immunotherapy. As used herein a mutation that can reduce antigen presentation potential is a mutation in the genome of a cell (e.g., a cancer cell) that reduce the ability of that cell to present one or more antigens on its surface (e.g., as compared to a cell that does not have that particular mutation in its genome). In some cases, one or more mutations in nucleic acid encoding an antigen presenting polypeptide (e.g., MHC class I polypeptides) can reduce the ability of that cell to present one or more antigens on its surface. Any appropriate genomic event can reduce the antigen presentation potential of a cell (e.g., cancer cell). Examples of genomic events that can reduce the antigen presentation potential of a cell (e.g., cancer cell) can include, without limitation, a loss of homozygosity of an HLA locus. For example, a cancer cell whose genome has a homozygous loss of at least one HLA class I locus (e.g., a homozygous loss of HLA-B) can have a reduced antigen presentation potential. In some cases, a genomic event that can reduce the antigen presentation potential of a cell (e.g., a cancer cell) can be as described in Example 1. In some cases, a genomic event that can reduce the antigen presentation potential of a cell (e.g., a cancer cell) can be as described in Table 11.

Any appropriate method can be used to determine the ability of one or more cells (e.g., one or more cancer cells) from a mammal to present one or more antigens. In some cases, immunohistochemistry techniques, whole exome sequencing, targeted next generation sequencing, or expression analyses can be used to determine the ability of one or more cells from a mammal to present one or more antigens.

In some cases, the presence of a smoking-related mutational signature in one or more cells (e.g., one or more cancer cells) from a mammal can be used to identify that mammal as being likely to be responsive to a cancer immunotherapy. As used herein, a smoking-related mutational signature includes one or more (e.g., one, two, three, four, five, six, or more) mutations that are C>A transversions in the genome of a cell (e.g., a cancer cell) from a mammal. A smoking-related mutational signature can include one or more C>A transversions in any appropriate nucleic acid sequence within the genome of a cell. In some cases, a C>A transversion can be in a coding sequence (or a regulatory region that can control expression of that coding sequence). In some cases, a C>A transversion can be a in a non-coding sequence. In some cases, a smoking-related mutational signature can be as described in Example 1.

Any appropriate method can be used to determine the presence or absence of a smoking-related mutational signature in one or more cells (e.g., one or more cancer cells) from a mammal. In some cases, the presence or absence of a C>A transversion can be detected using sequencing techniques (e.g., PCR-based sequencing such as Next-Generation PCR-based sequencing and Sanger sequencing), DNA hybridization techniques, and/or restriction enzyme digestion methods.

In some cases, a cTMB (and, optionally, the presence of one or more mutations in one or more nucleic acid sequences encoding a RTK polypeptide, the ability to present one or more antigens, and/or the presence of a smoking-related mutational signature) in one or more cells (e.g., one or more cancer cells) from a mammal can be used to determine whether or not that mammal is likely to respond to a particular cancer treatment (e.g., a cancer immunotherapy). For example, a cTMB including the presence of one or more particular mutations in one or more particular nucleic acid sequences encoding a RTK polypeptide, the ability to present one or more antigens, and/or the presence of a smoking-related mutational signature in one or more cells (e.g., one or more cancer cells) from a mammal can be used to determine whether or not that mammal is likely to respond to a cancer immunotherapy.

When a cTMB (and, optionally, the presence of one or more mutations in one or more nucleic acid sequences encoding a RTK polypeptide, the ability to present one or more antigens, and/or the presence of a smoking-related mutational signature) in one or more cells (e.g., one or more cancer cells) from a mammal can be used to determine that a cancer is likely to respond to a cancer immunotherapy, the cTMB can include any appropriate one or more mutations. For example, a cTMB and, optionally, the presence of one or more mutations in one or more nucleic acid sequences encoding a RTK polypeptide, the ability to present one or more antigens, and/or the presence of a smoking-related mutational signature can be used to determine that a cancer is likely to respond to a cancer immunotherapy. In some cases, a cTMB that can be used as described herein to determine that a cancer is likely to respond to a cancer immunotherapy can be a cTMB that includes one or more mutations in a nucleic acid that can encode ARID1A, one or more inactivating mutations in nucleic acid that can encode KEAP1, and/or one or more C>A transversions (e.g., a smoking-related mutational signature).

When a cTMB (and, optionally, the presence of one or more mutations in one or more nucleic acid sequences encoding a RTK polypeptide, the ability to present one or more antigens, and/or the presence of a smoking-related mutational signature) in one or more cells (e.g., one or more cancer cells) from a mammal can be used to determine that a cancer is not likely to respond to a cancer immunotherapy, the cTMB can include any appropriate one or more mutations. For example, a cTMB and, optionally, the presence of one or more mutations in one or more nucleic acid sequences encoding a RTK polypeptide, the ability to present one or more antigens, and/or the presence of a smoking-related mutational signature can be used to determine that a cancer is not likely to respond to a cancer immunotherapy. In some cases, a cTMB that can be used as described herein to determine that a cancer is not likely to respond to a cancer immunotherapy can be a cTMB that includes one or more activating mutations in nucleic acid that can encode EGFR, one or more activating mutations in nucleic acid that can encode ERBB2, one or more activating mutations in nucleic acid that can encode MET, one or more activating mutations in nucleic acid that can encode FGFR1, one or more activating mutations in nucleic acid that can encode IGF1R, one or more activating mutations in nucleic acid that can encode MDM2/MDM4, and/or a homozygous loss of at least one HLA class I locus. For example, a cTMB having a mutational signature that includes one or more activating point mutations in nucleic acid encoding EGFR, one or more activating point mutations in nucleic acid encoding ERBB2, amplification of nucleic acid encoding MET, amplification of nucleic acid encoding FGFR1, amplification of nucleic acid encoding IGF1R, one or more activating point mutations in nucleic acid encoding MDM2/MDM4, and homozygous loss of at least one HLA class I locus can be used to determine that a cancer is not likely to respond to a cancer immunotherapy.

A mammal (e.g., a human) having a cancer can be administered, or instructed to self-administer, any one or more (e.g., 1, 2, 3, 4, 5, 6, or more) cancer treatments. A cancer treatment can include any appropriate cancer treatment. In some cases, a cancer treatment can include surgery. In some cases, a cancer treatment can include radiation therapy. In some cases, a cancer treatment can include administration of a pharmacotherapy such as a chemotherapy, hormone therapy, targeted therapy, and/or cytotoxic therapy. Examples of cancer treatments include, without limitation, administration of one or more receptor tyrosine kinase inhibitors (e.g., erlotinib), administration of one or more PD1/PD-L1 inhibitors (e.g., nivolumab, pembrolizumab, atezolizumab, avelumab, and durvalumab), administration of one or more immunotherapies (e.g., alemtuzumab, ipilimumab, nivolumab, ofatumumab, and rituximab), administration of one or more platinum compounds (e.g., a cisplatin or carboplatin), administration of one or more taxanes (e.g., paclitaxel, docetaxel, or an albumin bound paclitaxel such as nab-paclitaxel), administration of altretamine, administration of capecitabine, administration of cyclophosphamide, administration of etoposide (vp-16), administration of gemcitabine, administration of ifosfamide, administration of irinotecan (cpt-11), administration of liposomal doxorubicin, administration of melphalan, administration of pemetrexed, administration of topotecan, administration of vinorelbine, administration of one or more luteinizing-hormone-releasing hormone (LHRH) agonists (such as goserelin and leuprolide), administration of one or more anti-estrogen therapies (such as tamoxifen), administration of one or more aromatase inhibitors (such as letrozole, anastrozole, and exemestane), administration of one or more angiogenesis inhibitors (such as bevacizumab), administration of one or more poly(ADP)-ribose polymerase (PARP) inhibitors (such as olaparib, rucaparib, and niraparib), administration of external beam radiation therapy, administration of brachytherapy, administration of radioactive phosphorus, and administration of any combinations thereof.

In cases where a mammal (e.g., a human) is identified as having a cancer that is likely to be responsive to a cancer immunotherapy based, at least in part, on the cTMB of the sample from the mammal, the mammal can be treated with one or more (e.g., 1, 2, 3, 4, 5, 6, or more) cancer immunotherapies. In some cases, a cancer immunotherapy can be a cellular immunotherapy (e.g., a dendritic cell therapy or a chimeric antigen receptor (CAR)-T cell therapy). In some cases, a cancer immunotherapy can be an antibody therapy (e.g., a monoclonal antibody therapy). In some cases, a cancer immunotherapy can be a cytokine therapy (e.g., interferon therapy or interleukin therapy). In some cases, a cancer immunotherapy can activate one or more cell death mechanisms (e.g., antibody-dependent cell-mediated cytotoxicity (ADCC) or the complement system). In some cases, a cancer immunotherapy can target one or more (e.g., 1, 2, 3, 4, 5, 6, or more) immune checkpoint molecules. An immune checkpoint molecule can be an inhibitory checkpoint molecule. Examples of immune checkpoint molecules that can be targeted by a cancer immunotherapy can include, without limitation, cytotoxic T-lymphocyte-associated protein 4 (CTLA4, also known as cluster of differentiation 152 (CD152)), programmed cell death protein 1 (PD-1, also known as cluster of differentiation 279 (CD279)), and programmed death-ligand 1 (PD-L1, also known as cluster of differentiation 274 (CD274) and B7 homolog 1 (B7-H1)). Examples of cancer immunotherapies that can be administered to a mammal identified as having a cancer that is likely to be responsive to a cancer immunotherapy based, at least in part, on the cTMB of a sample from the mammal can include, without limitation, alemtuzumab, atezolizumab, avelumab, ipilimumab, ofatumumab, nivolumab, pembrolizumab, rituximab, and durvalumab.

In cases where a mammal (e.g., a human) is identified as having a cancer that is likely to be responsive to a cancer immunotherapy based, at least in part, on the cTMB of the sample from the mammal, the mammal can be treated with a cancer immunotherapy and also can be administered any one or more (e.g., 1, 2, 3, 4, 5, 6, or more) additional cancer treatments (e.g., one or more cancer treatments that are not cancer immunotherapies). A cancer treatment can include any appropriate cancer treatment. A cancer treatment can include any appropriate cancer treatment. In some cases, a cancer treatment can include surgery. In some cases, a cancer treatment can include radiation therapy. In some cases, a cancer treatment can include administration of a pharmacotherapy such as a chemotherapy, hormone therapy, targeted therapy, and/or cytotoxic therapy. Examples of chemotherapeutic agents that can be administered to a mammal having a cancer can include, without limitation, pemetrexed, platinum-based compounds, taxanes, and combinations thereof.

In cases where a mammal having cancer is treated with one or more (e.g., 1, 2, 3, 4, 5, 6, or more) cancer immunotherapies and is treated with one or more (e.g., 1, 2, 3, 4, 5, 6, or more) additional cancer treatments (e.g., one or more cancer treatments that are not cancer immunotherapies), the one or more cancer immunotherapies and the one or more additional cancer treatments can be administered at the same time or independently. For example, one or more cancer immunotherapies can be administered first, and the one or more additional cancer treatments (e.g., one or more cancer treatments that are not cancer immunotherapies) administered second, or vice versa.

The invention will be further described in the following examples, which do not limit the scope of the invention described in the claims.

### EXAMPLES

### Example 1: Genomic Drivers of Response to Immune Checkpoint Blockade in Non-Small Cell Lung Cancer

This Example describes an integrated approach where an improved measure for TMB, corrected for tumor purity, is combined with genomic alterations in RTK genes, genome-wide mutational signatures, and HLA class I genetic variation to capture the multifaceted nature of the tumor-immune system crosstalk and more accurately predict outcome for immune checkpoint blockade.

Results

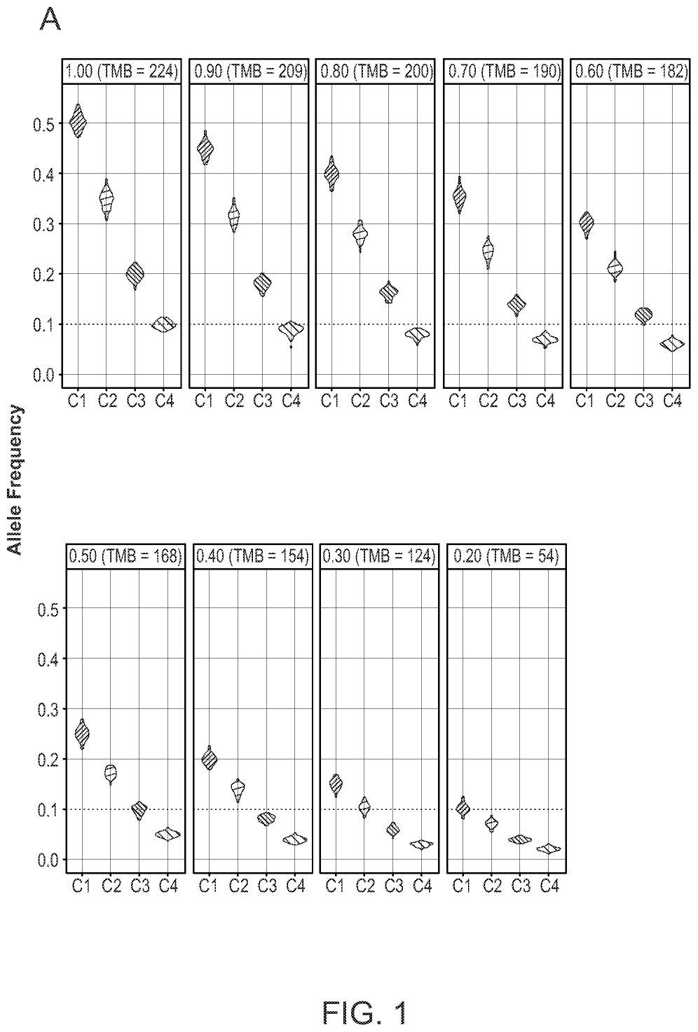

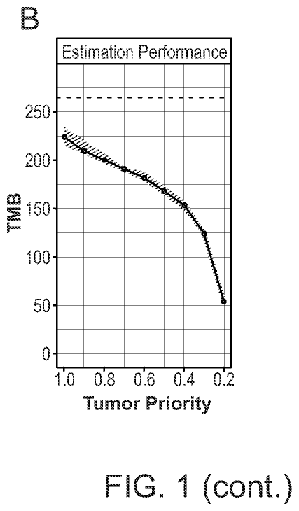

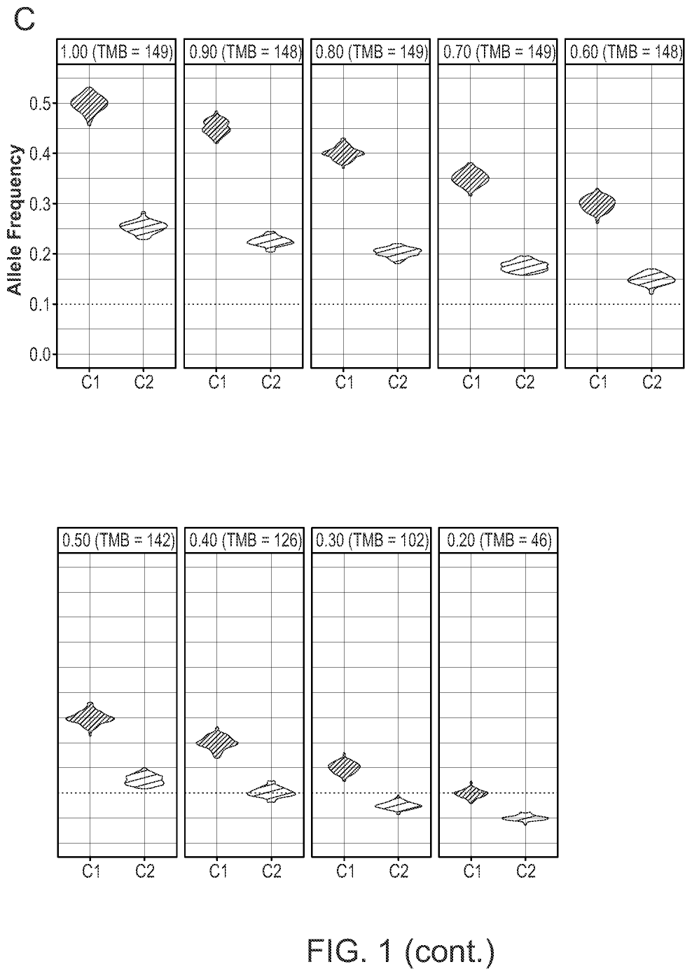

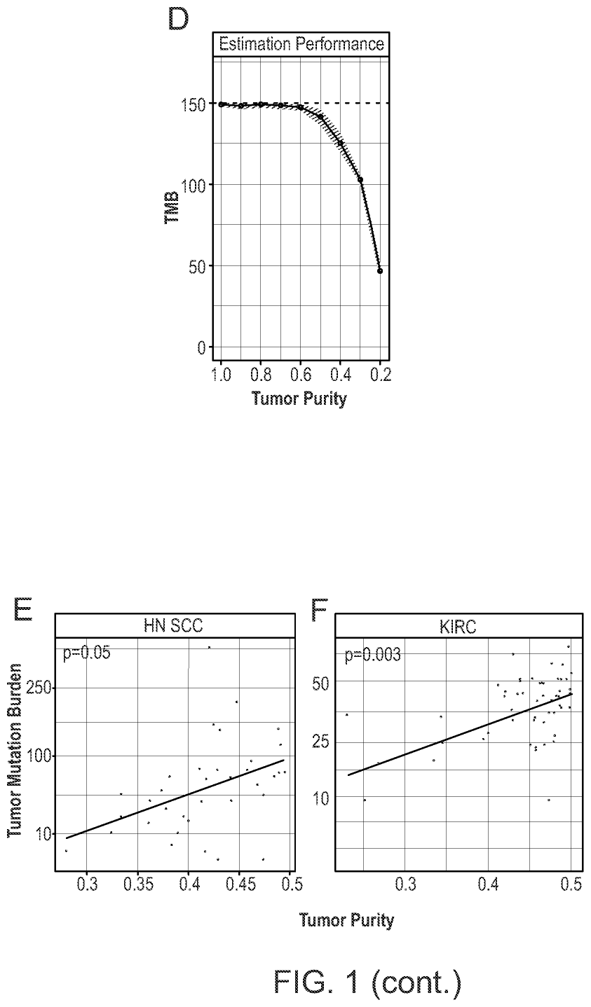

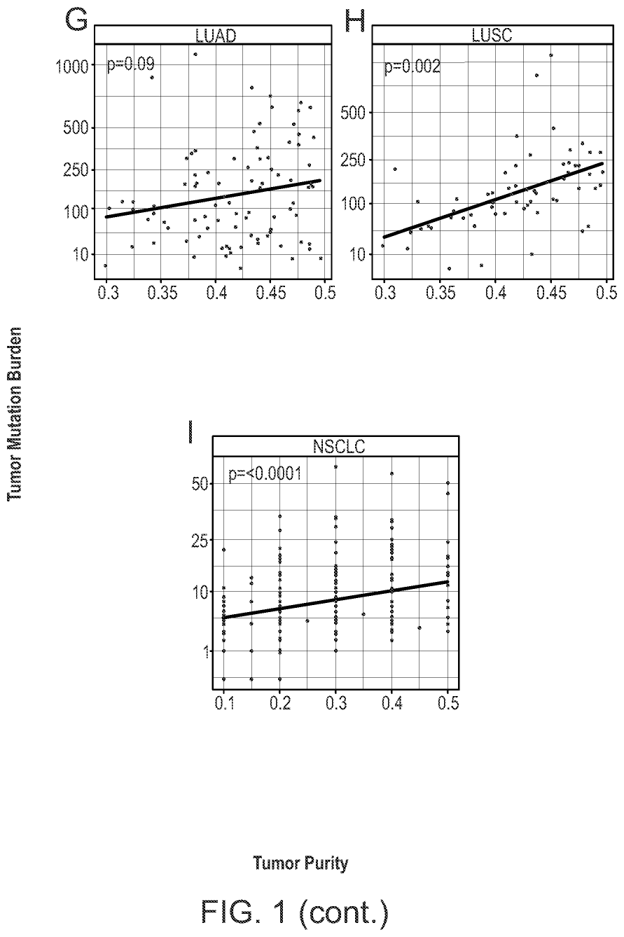

TMB is an emerging predictive biomarker of response to immune checkpoint blockade, however its broad implementation in clinical decision making has been hindered by complexities with establishing a robust predictive power. Low tumor purity, mainly due to sampling, may greatly affect TMB assessments, resulting in falsely low TMB in low tumor cellularity samples, especially for tumors with a higher fraction of subclonal mutations. Furthermore, the estimation of tumor purity itself may be challenging as pathologic assessments are frequently imprecise and have limited reproducibility (Viray et al., Archives of pathology & laboratory medicine 137:1545-1549 (2013)). To determine tumor purity for cohorts 1 and 2, both a mutant allele frequency based and a copy-number based approach were employed. To determine the tumor purity needed to accurately determine TMB in the setting of different clonal composition backgrounds, simulation analyses were performed and the tumor purity required to establish reliable TMB assessments was determined, and that TMB also depends on intratumoral clonal heterogeneity (FIG. 1). On the lower spectrum of tumor purity, TMBs of clonally heterogeneous TMB-high and clonally homogeneous TMB-low tumors become indiscernible, underlining the need to correct TMB for tumor purity.

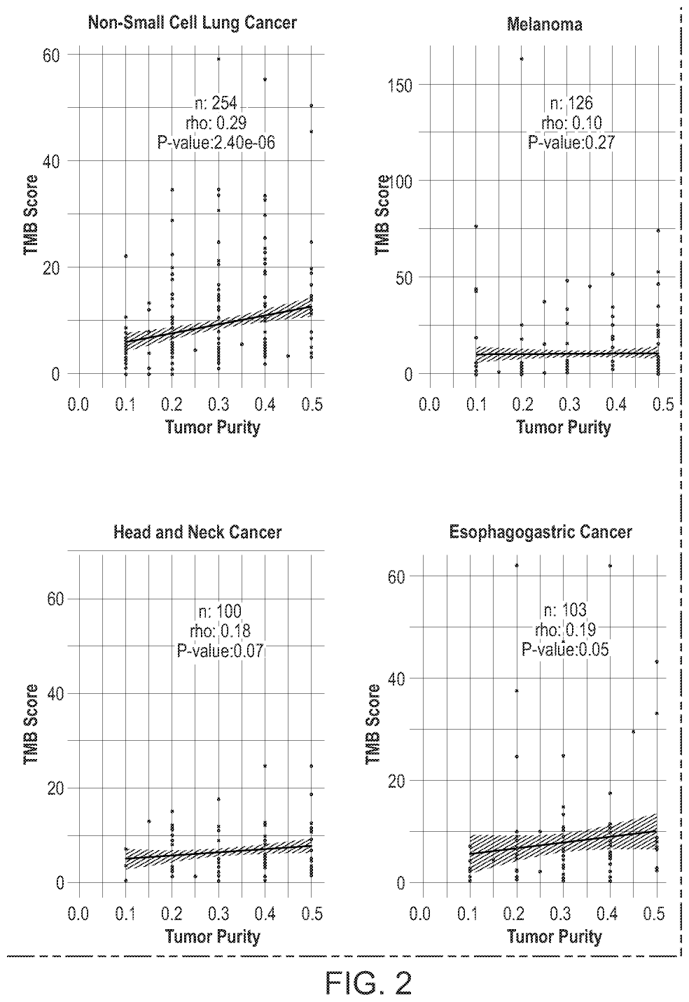

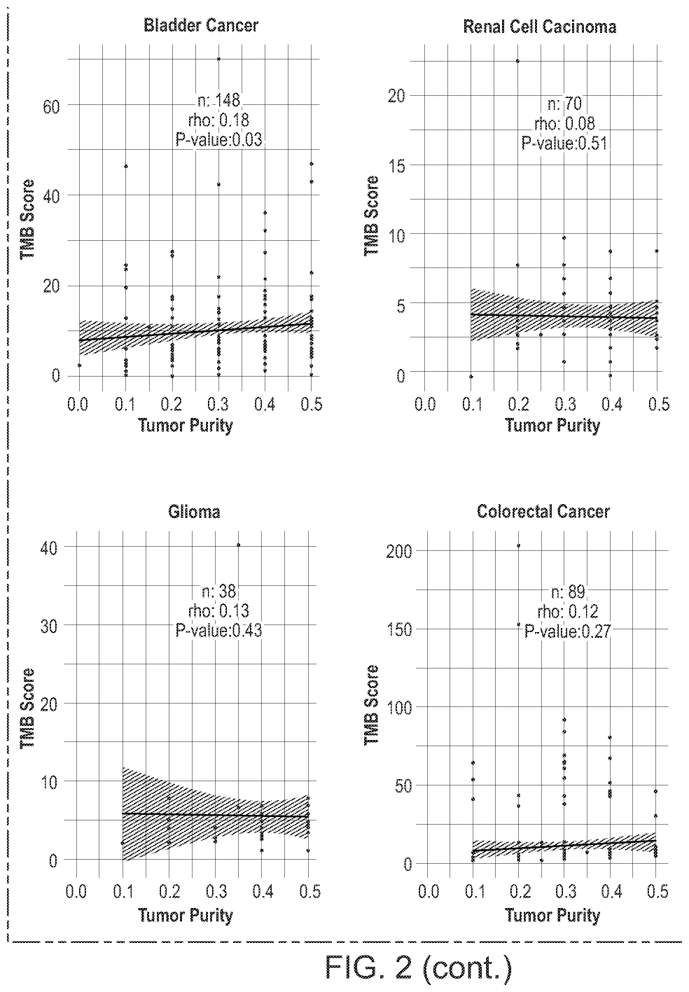

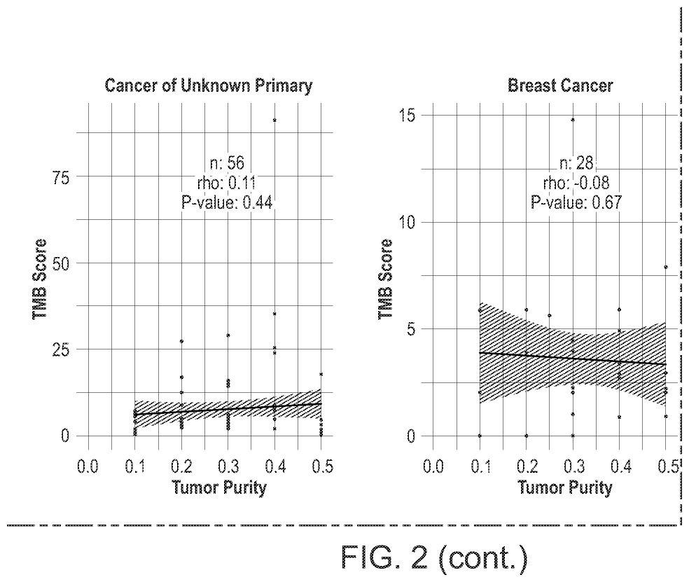

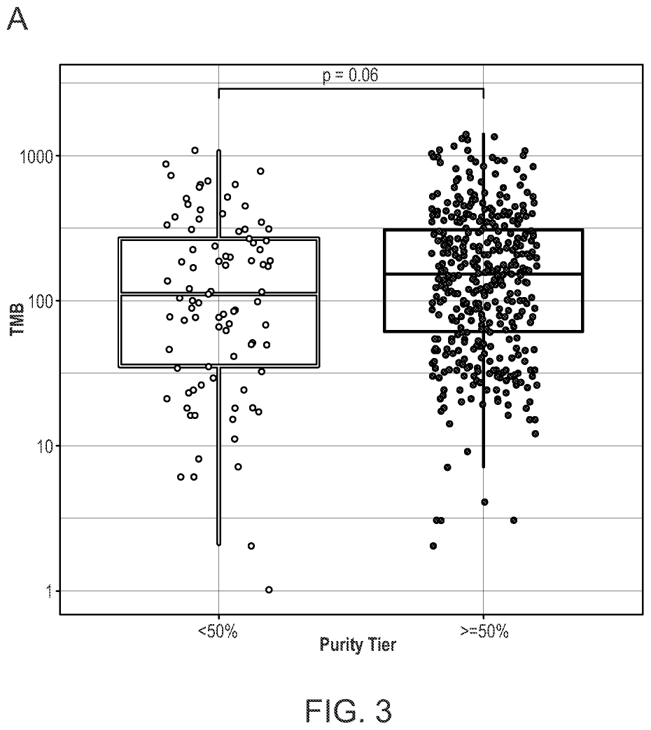

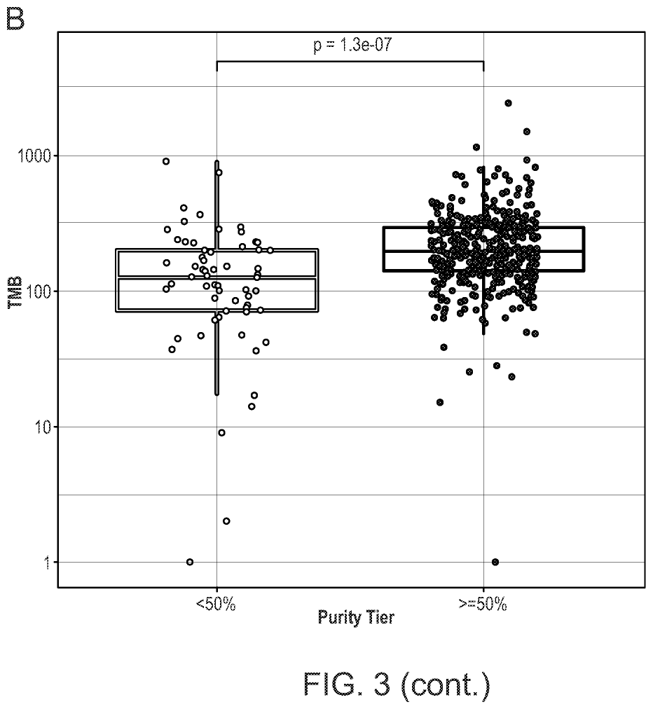

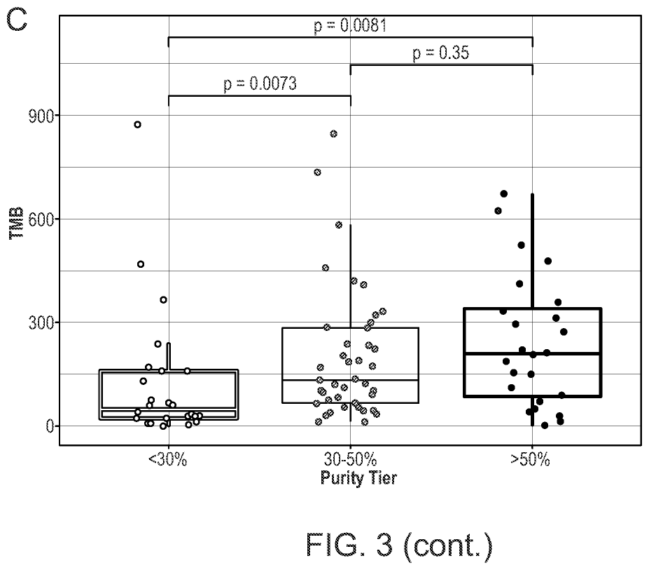

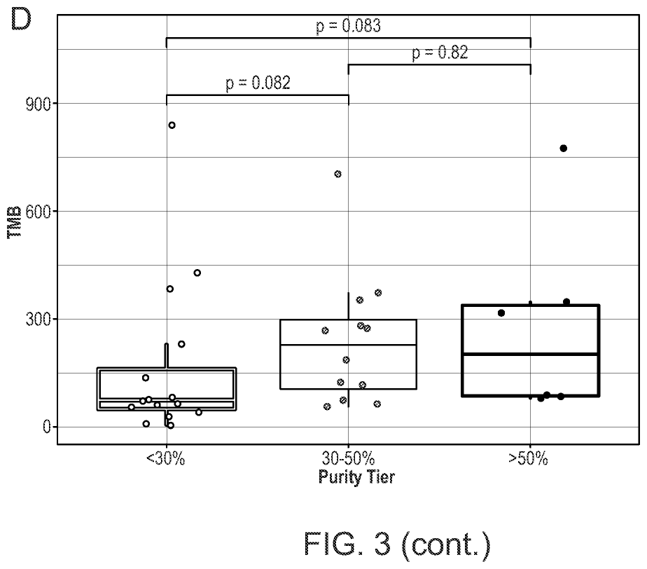

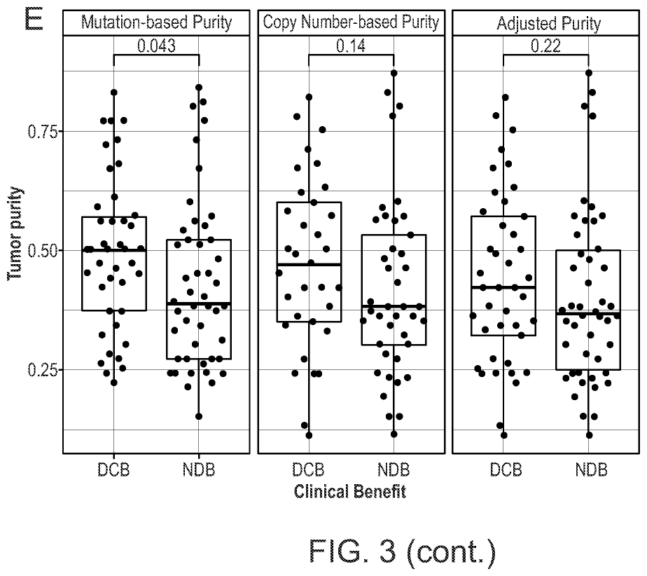

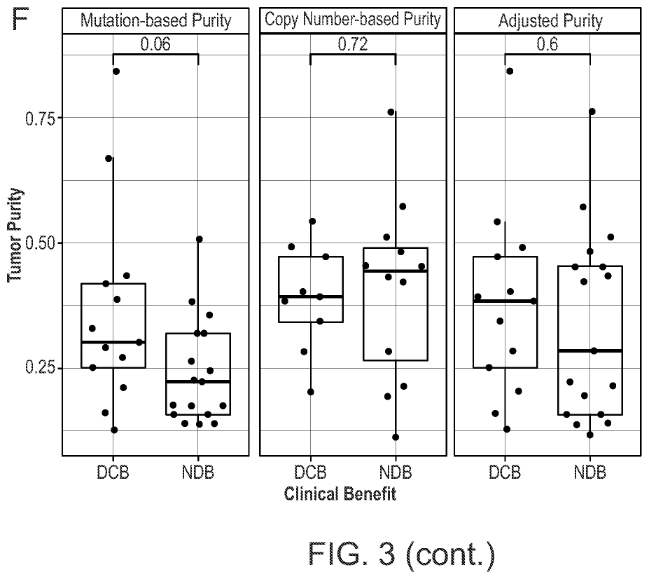

To substantiate these findings, tumor whole exome sequencing data from 3,788 TCGA samples from 7 tumor types (bladder carcinoma, breast carcinoma, colon adenocarcinoma, head and neck squamous cell carcinoma, kidney clear cell carcinoma, NSCLC and melanoma) were analyzed and a correlation between TMB and tumor purity was found, with a lower number of alterations observed in samples with low tumor purity (FIG. 1). Focusing on lung adenocarcinomas (LUAD, n=493) and squamous cell carcinomas (LUSC, n=464), it was found that the correlation between TMB and tumor purity was particularly pronounced for samples with tumor purity below 50% (Pearson's R=0.18, p=0.09 and R=0.39, p=0.002 in LUAD and LUSC respectively; FIG. 1). Given that targeted NGS approaches enable deeper sequencing coverage and may therefore mitigate the effect of tumor purity on analysis of low tumor purity highly clonally heterogeneous tumors, the correlation between tumor purity and TMB was evaluated in a large cohort of tumors sequenced with targeted next-generation sequencing (Samstein et al., Nature genetics, doi:10.1038/s41588-018-0312-8 (2019)). A significant correlation between tumor purity and TMB estimates was identified, particularly in NSCLC (FIG. 2), suggesting that tumor purity remains a limiting factor for accurately estimating TMB even in the setting of higher sequencing depth. To further examine TMB and identify other biomarkers of response to ICB, whole exome sequencing was performed on 104 matched tumor/normal pairs from NSCLC patients treated with ICB. Eighty-nine cases that passed strict quality control measures (Methods) were further analyzed (cohort 1, Tables 1-3) and a published cohort of 34 NSCLC patients treated with anti-PD1 blockadel (cohort 2) was analyzed for independent validation. In the two immunotherapy treated NSCLC cohorts, the correlation between TMB and tumor purity was particularly pronounced for tumor purity less than 30% (p=0.008 and P=0.08 for overall comparisons of TMB across tumor purity tiers for cohort 1 and cohort 2 respectively; FIG. 3). These findings suggest that observed TMB values may largely deviate from the true TMB in low purity tumors.

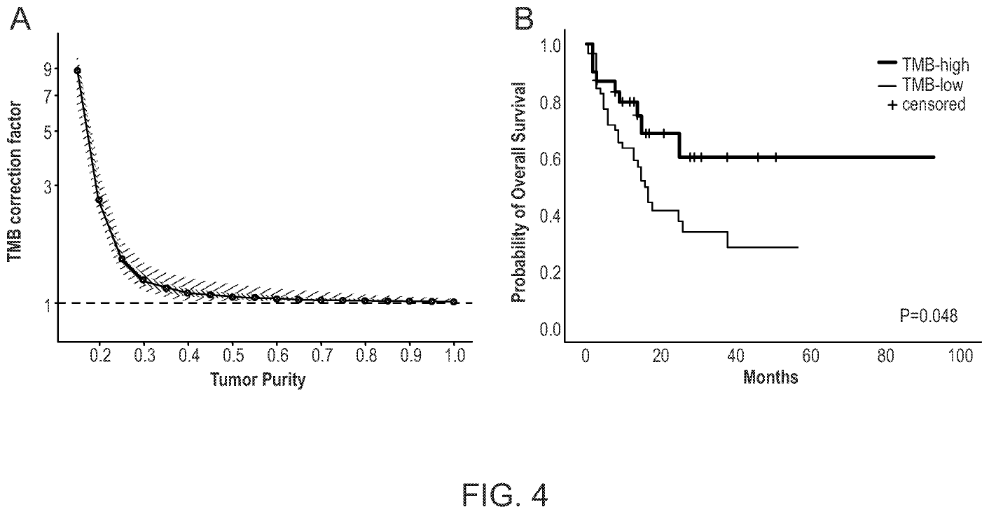

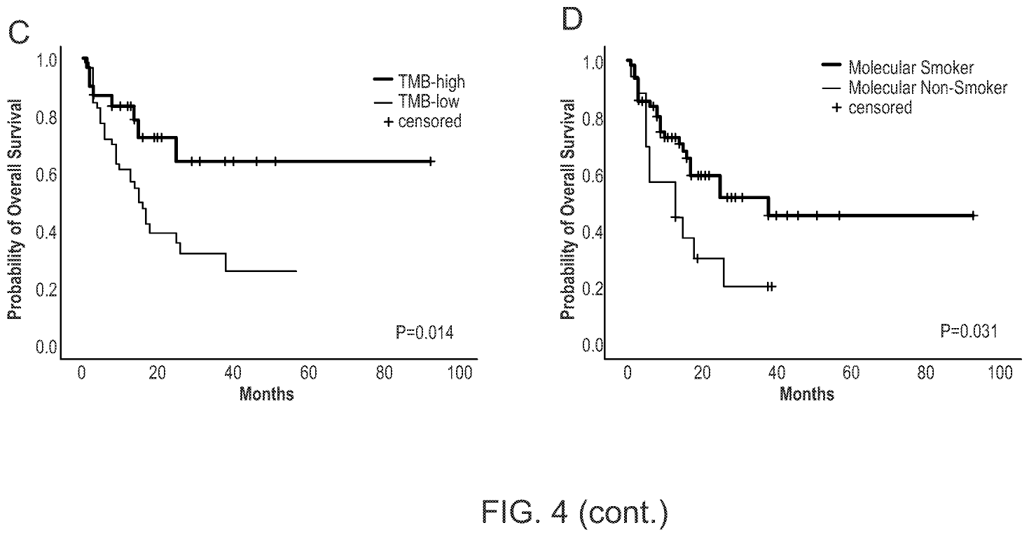

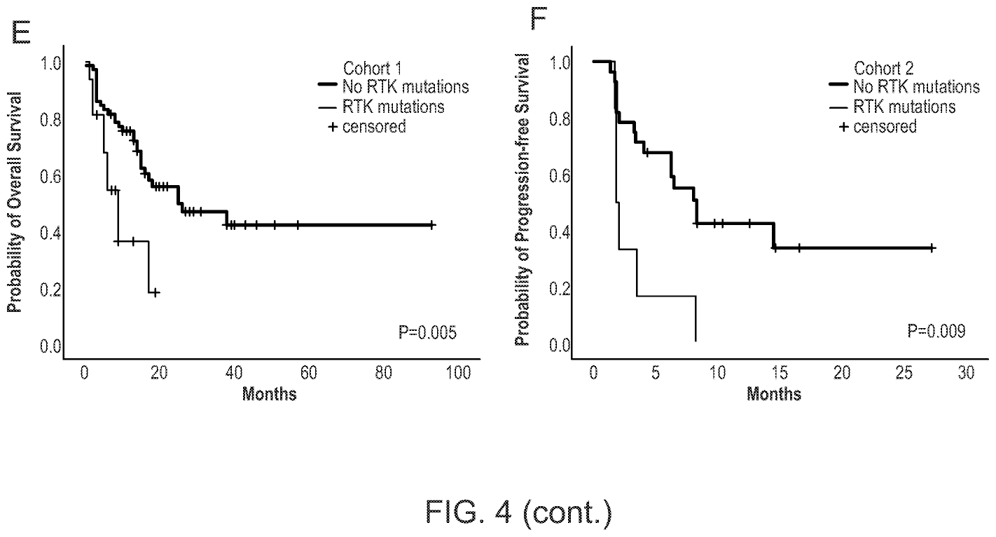

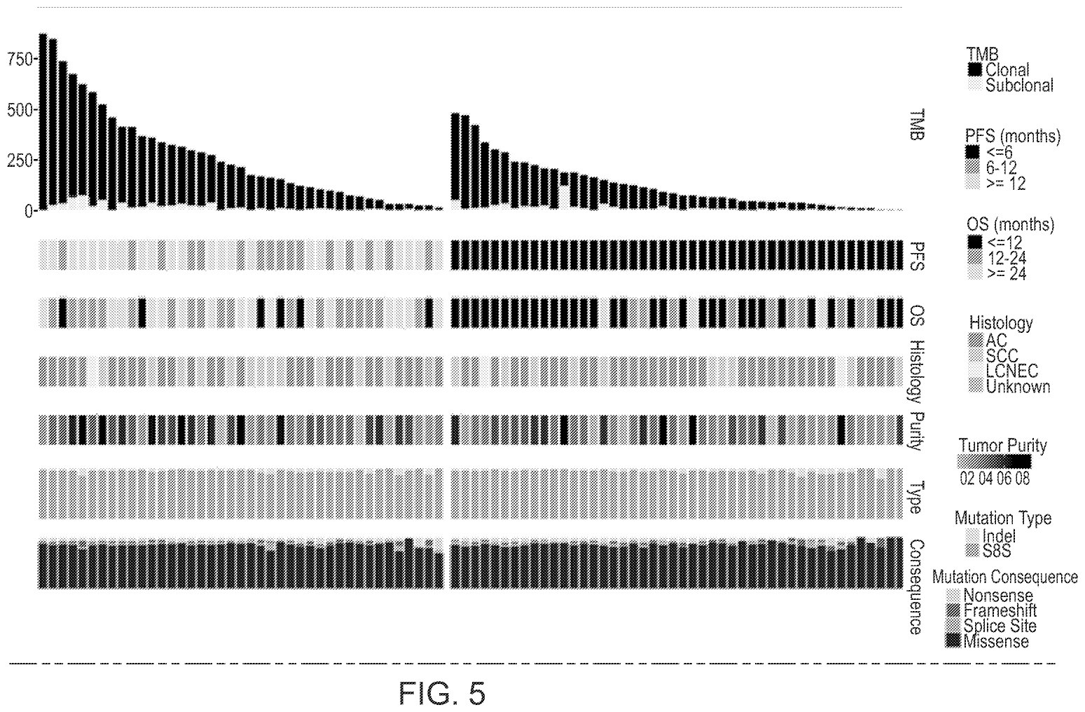

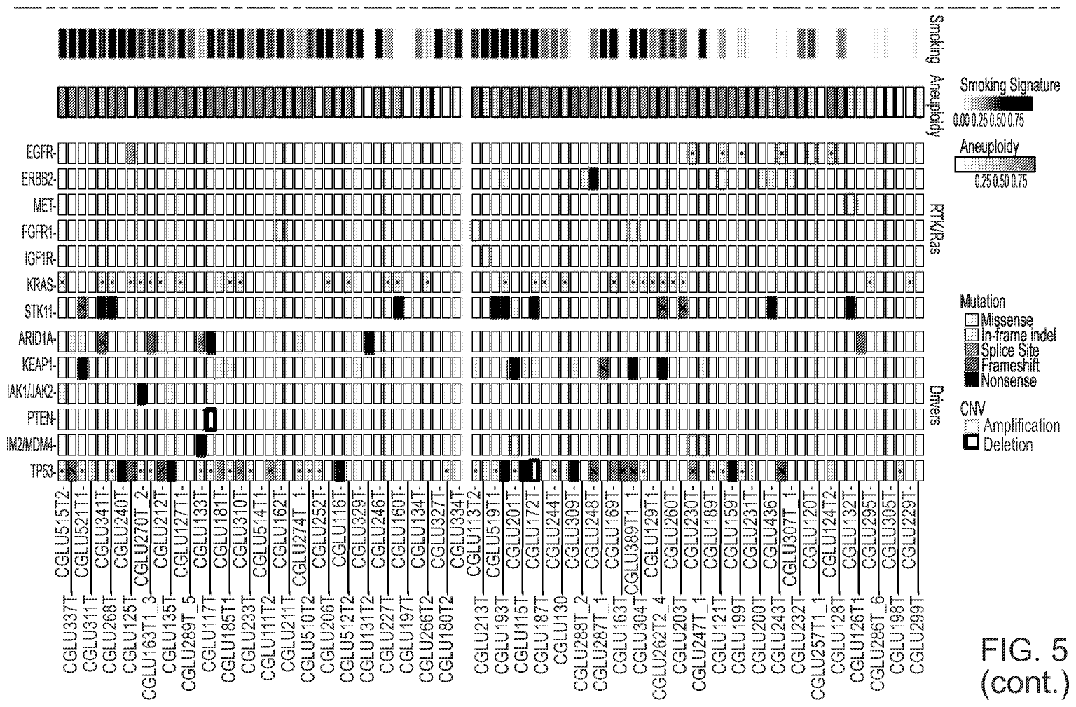

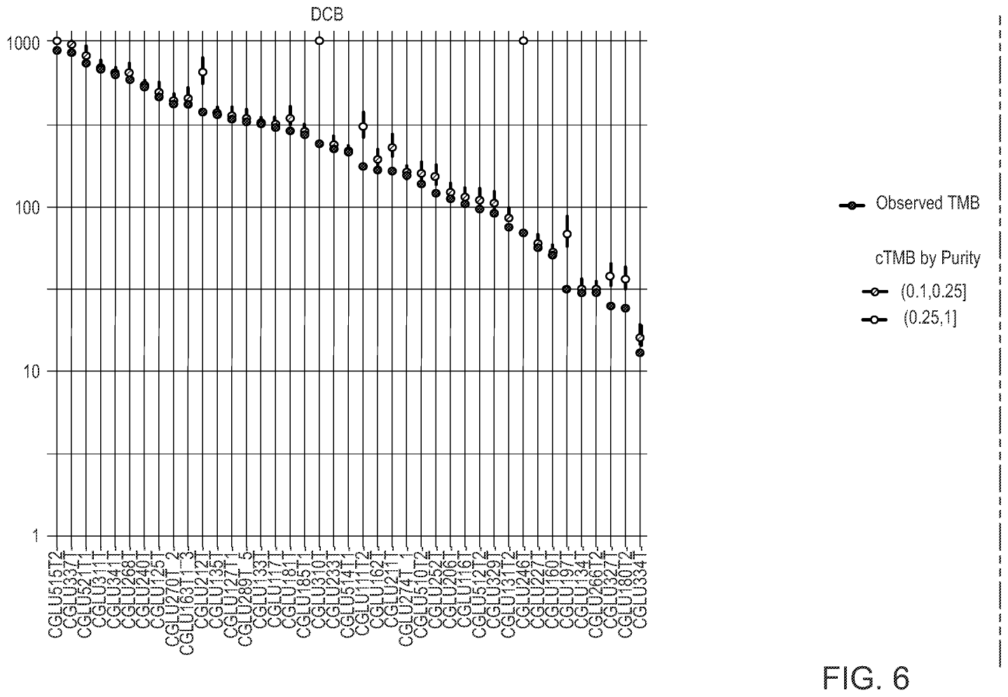

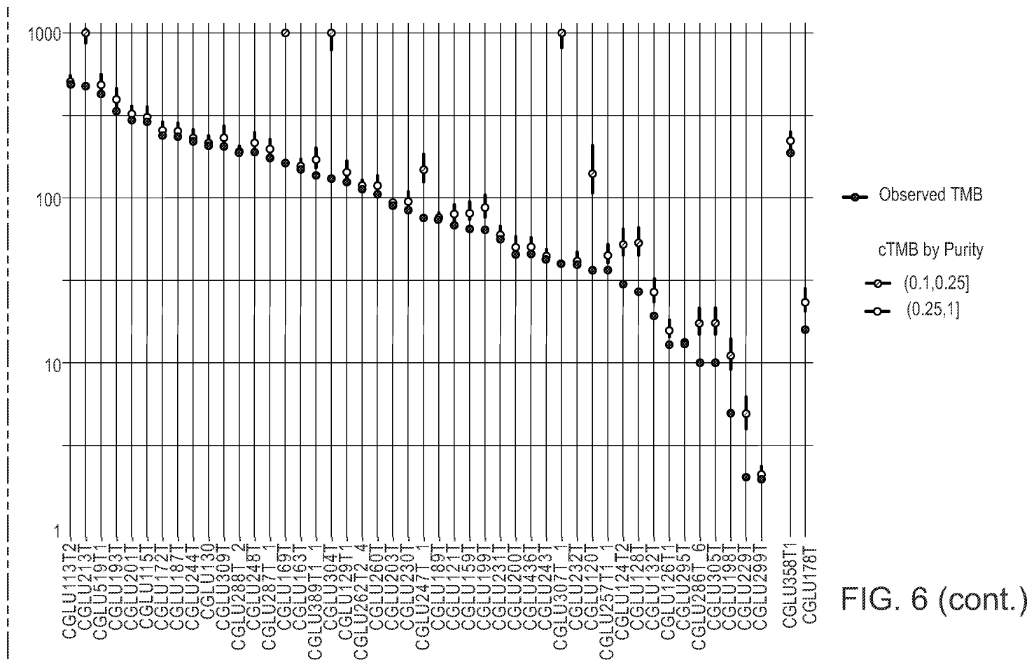

To overcome this limitation of TMB measurements, an approach was developed to estimate corrected TMB values (cTMB) for each tumor based on tumor purity. First, 20,000 tumors were simulated with various levels of intra-tumoral heterogeneity, TMB, and depth of coverage using a reference set from TCGA. In silico dilutions of these simulated tumors were then used to model the observed TMB resulting from characterization of each simulated tumor sample at various levels of tumor purity. For each simulated tumor a correction factor was generated for different purity tiers (FIG. 4A and Table 4). An analysis of the observed TMB in cohort 1 revealed that patients with durable clinical benefit to ICB had significantly higher observed tumor mutation burden compared to patients with non-durable clinical benefit (Mann-Whitney p=0.002, FDR-adjusted p=0.012, FIG. 5, Table 5). There was a substantial overlap in the range of observed TMB between the two groups (FIG. 5), and observed TMB only marginally predicted overall survival (log rank p=0.048, FIG. 4B). Using the developed correction factors for different purity tiers, cTMB values were determined for the tumors in the cohort (Table 4). Corrected TMB more accurately predicted overall survival (log rank p=0.014, FIG. 4C), suggesting that the observed TMB may be largely underestimated in low tumor purity samples and result in misclassification of patients with these tumors (FIG. 6).

TABLE 4
Correction factor for observed TMB by tumor purity tier

| Tumor Purity | Correction Factor | Correction Factor CI (95%) |
|---|---|---|
| 1 | 1.01 | (1.00-1.07) |
| 0.99 | 1.02 | (1.00-1.07) |
| 0.98 | 1.02 | (1.00-1.07) |
| 0.97 | 1.02 | (1.00-1.07) |
| 0.96 | 1.02 | (1.00-1.07) |
| 0.95 | 1.02 | (1.00-1.07) |
| 0.94 | 1.02 | (1.00-1.07) |
| 0.93 | 1.02 | (1.00-1.07) |
| 0.92 | 1.02 | (1.00-1.07) |
| 0.91 | 1.02 | (1.00-1.08) |
| 0.9 | 1.02 | (1.00-1.08) |
| 0.89 | 1.02 | (1.00-1.08) |
| 0.88 | 1.02 | (1.00-1.08) |
| 0.87 | 1.02 | (1.00-1.08) |
| 0.86 | 1.02 | (1.00-1.08) |
| 0.85 | 1.02 | (1.00-1.08) |
| 0.84 | 1.02 | (1.00-1.09) |
| 0.83 | 1.02 | (1.00-1.09) |
| 0.82 | 1.02 | (1.00-1.09) |
| 0.81 | 1.02 | (1.00-1.09) |
| 0.8 | 1.02 | (1.00-1.09) |
| 0.79 | 1.02 | (1.00-1.10) |
| 0.78 | 1.02 | (1.00-1.10) |
| 0.77 | 1.03 | (1.00-1.10) |
| 0.76 | 1.03 | (1.00-1.10) |
| 0.75 | 1.03 | (1.00-1.10) |
| 0.74 | 1.03 | (1.00-1.11) |
| 0.73 | 1.03 | (1.00-1.11) |
| 0.72 | 1.03 | (1.00-1.11) |
| 0.71 | 1.03 | (1.00-1.11) |
| 0.7 | 1.03 | (1.00-1.12) |
| 0.69 | 1.03 | (1.00-1.12) |
| 0.68 | 1.03 | (1.00-1.12) |
| 0.67 | 1.03 | (1.00-1.13) |
| 0.66 | 1.04 | (1.00-1.13) |
| 0.65 | 1.04 | (1.00-1.13) |
| 0.64 | 1.04 | (1.00-1.14) |
| 0.63 | 1.04 | (1.00-1.14) |
| 0.62 | 1.04 | (1.00-1.14) |
| 0.61 | 1.04 | (1.00-1.15) |
| 0.6 | 1.04 | (1.00-1.15) |
| 0.59 | 1.05 | (1.00-1.15) |
| 0.58 | 1.05 | (1.01-1.16) |
| 0.57 | 1.05 | (1.01-1.16) |
| 0.56 | 1.05 | (1.01-1.17) |
| 0.55 | 1.05 | (1.01-1.17) |
| 0.54 | 1.06 | (1.01-1.18) |
| 0.53 | 1.06 | (1.01-1.18) |
| 0.52 | 1.06 | (1.01-1.19) |
| 0.51 | 1.06 | (1.01-1.19) |
| 0.5 | 1.06 | (1.01-1.20) |
| 0.49 | 1.07 | (1.01-1.20) |
| 0.48 | 1.07 | (1.01-1.21) |
| 0.47 | 1.07 | (1.01-1.22) |
| 0.46 | 1.08 | (1.02-1.22) |
| 0.45 | 1.08 | (1.02-1.23) |
| 0.44 | 1.09 | (1.02-1.24) |
| 0.43 | 1.09 | (1.02-1.25) |
| 0.42 | 1.1 | (1.02-1.26) |
| 0.41 | 1.1 | (1.03-1.26) |
| 0.4 | 1.11 | (1.03-1.27) |
| 0.39 | 1.12 | (1.03-1.29) |
| 0.38 | 1.12 | (1.04-1.30) |
| 0.37 | 1.13 | (1.04-1.31) |
| 0.36 | 1.14 | (1.05-1.33) |
| 0.35 | 1.15 | (1.05-1.34) |
| 0.34 | 1.17 | (1.06-1.37) |
| 0.33 | 1.19 | (1.08-1.39) |
| 0.32 | 1.21 | (1.09-1.42) |
| 0.31 | 1.23 | (1.10-1.44) |
| 0.3 | 1.25 | (1.11-1.47) |
| 0.29 | 1.3 | (1.15-1.54) |
| 0.28 | 1.35 | (1.19-1.62) |
| 0.27 | 1.41 | (1.23-1.69) |
| 0.26 | 1.46 | (1.27-1.76) |
| 0.25 | 1.51 | (1.31-1.83) |
| 0.24 | 1.74 | (1.48-2.15) |
| 0.23 | 1.96 | (1.65-2.47) |
| 0.22 | 2.19 | (1.81-2.79) |
| 0.21 | 2.41 | (1.98-3.11) |
| 0.2 | 2.63 | (2.15-3.42) |
| 0.19 | 3.87 | (2.93-5.69) |
| 0.18 | 5.1 | (3.70-7.96) |
| 0.17 | 6.34 | (4.48-10.23) |
| 0.16 | 7.57 | (5.25-12.50) |
| 0.15 | 8.81 | (6.03-14.77) |
| 0.14 | 17.57 | (9.55-2011.81) |
| 0.13 | 26.34 | (13.08-4008.86) |
| 0.12 | 35.1 | (16.61-6005.91) |
| 0.11 | 43.87 | (20.14-8002.95) |
| 0.1 | 52.63 | (23.67-10000.00) |

TABLE 5
Differences in clinical and genomic characteristics between responders and non-
responders.

| | DCB (n = 41) | NDB (n = 46) | | |
|---|---|---|---|---|
| Characteristic | mean ± SE | mean ± SE | p value | FDR p value |
| Age | 66.46 ± 1.48 | 64.04 ± 1.50 | 0.286 | 0.650 |
| Gender (female vs male) | | | 1.000 | 1.000 |
| Histotype | | | 0.460 | 0.868 |
| TMB | 270.41 ± 36.65 | 128.19 ± 18.18 | 0.002 | 0.012 |
| TMB (high vs low) | | | 0.003 | 0.015 |
| Clonal TMB | 293.94 ± 39.25 | 130.98 ± 18.40 | <0.001 | 0.005 |
| Clonal TMB (high vs low) | | | 0.001 | 0.008 |
| Fraction of Clonal Mutations (%) | 95 ± 1 | 92 ± 2 | 0.422 | 0.650 |
| Adjusted Tumor Purity (%) | 44 ± 3 | 40 ± 3 | 0.193 | 0.579 |
| Molecular Smoking Signature (%)* | 49 ± 5 | 28 ± 5 | 0.003 | 0.027 |
| Hotspot Mutations | 1.56 ± 0.26 | 1.30 ± 0.16 | 0.588 | 0.706 |
| RTK mutations (yes vs no) | | | <0.001 | 0.003 |
| Fraction of Genome with LOH (%) | 32 ± 2 | 29 ± 3 | 0.317 | 0.650 |
| Fraction of Genome with Allelic Imbalance (%) | 66 ± 3 | 58 ± 4 | 0.367 | 0.650 |
| Genome Entropy | 1.85 ± 0.08 | 1.62 ± 0.10 | 0.192 | 0.579 |
| Fit MANAs | 18.51 ± 3.19 | 7.63 ± 1.23 | 0.012 | 0.050 |
| HLA class I germline alleles | | | 0.920 | 0.920 |
| HLA class I tumor alleles | | | 0.483 | 0.669 |
| Maximal germline HLA heterozygosity (yes vs no) | | | 1.000 | 1.000 |

*only tumors with a TMB equal or greater than 20 were included,
**fraction of genome with LOH, fraction of genome with allelic imbalance, entropy, clonal TMB, fraction of clonal mutations were calculated only for tumors with succeful copy number analyses (n = 74). Differences in continuous variables were assessed with the Mann-Whitney test, differences in categorical variables were assessed with the Fisher's exact test and differences between nominal variables were assessed with chi square, followed by FDR correction.

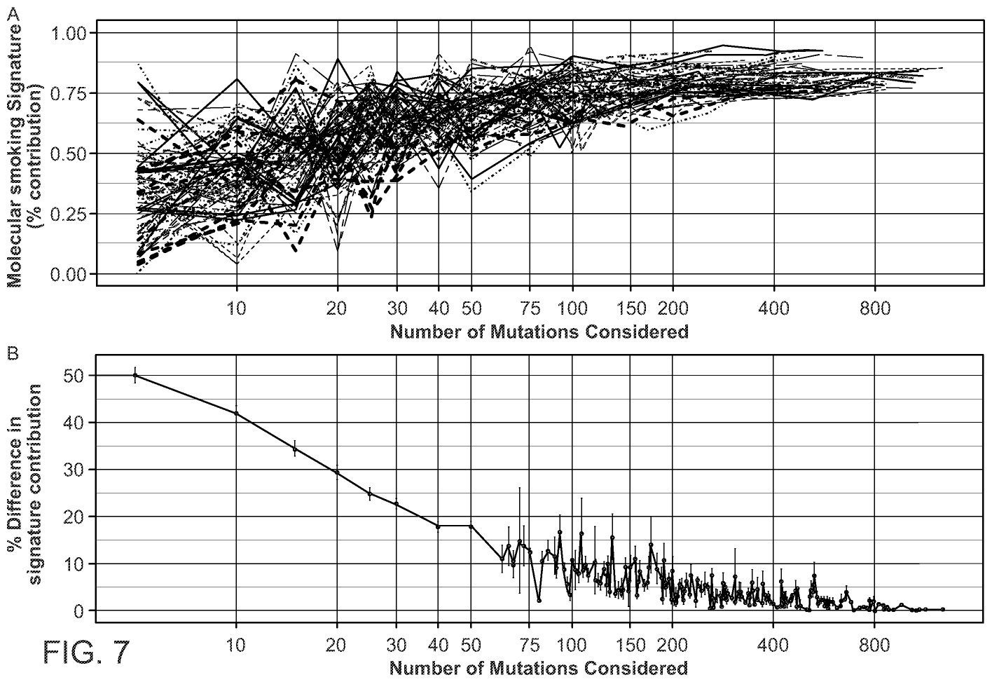

The approach was further refined by interrogating mutational signatures as smoking-related C>A transversions have been identified in NSCLC patients with clinical benefit from ICB (Miao et al., Nature genetics 50:1271-1281 (2018); and Forde et al., The New England journal of medicine, 378:1976-1986 (2018)). The number of mutations needed to accurately estimate the contribution of the C>A rich molecular smoking signature were evaluated. In silico dilution experiments of whole exome mutational profiles of 985 TCGA NSCLC tumors were performed and it was found that a minimum of 20 non-synonymous mutations would be required to predict the presence of a dominant smoking signature (FIG. 7). An analysis of tumor samples with at least 20 mutations revealed an enrichment of the molecular smoking signature in patients with durable clinical benefit (Mann-Whitney p=0.003, FDR-adjusted p=0.027, FIG. 5, Table 5). The molecular smoking signature more accurately predicted overall survival than observed TMB (log rank p=0.031, FIG. 4B), suggesting that the smoking-associated mutational processes are the likely cause of high mutation load and therefore, for samples with low tumor purity, mutational signatures could serve as a proxy for TMB.

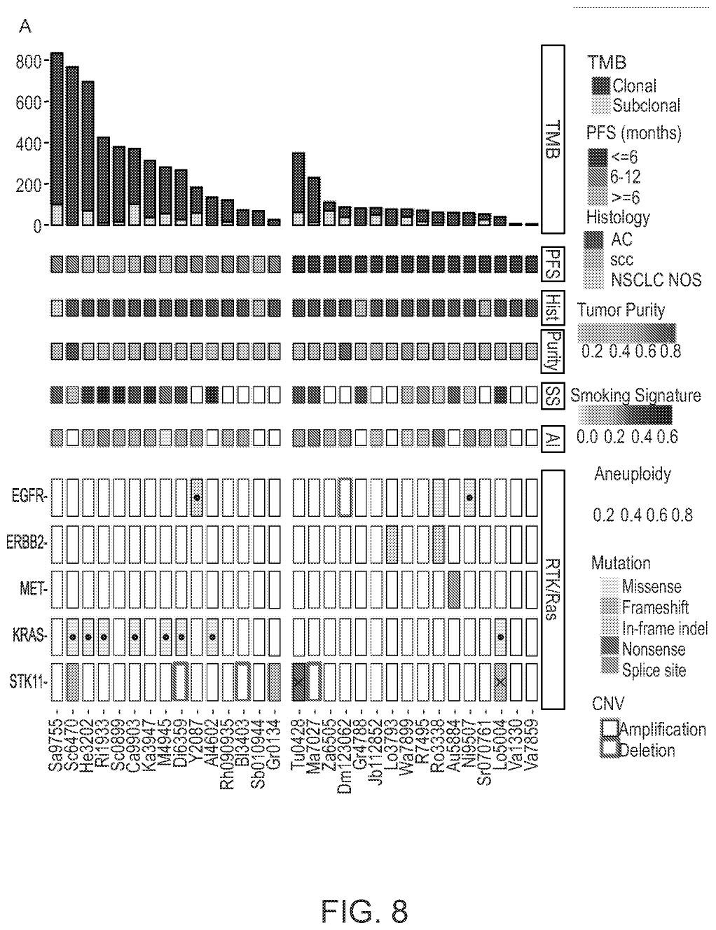

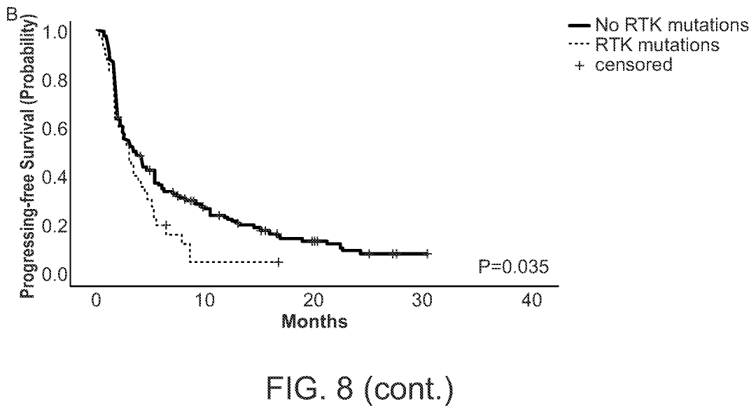

Genomic alterations in driver genes that were selectively associated with responding or non-responding tumors after accounting for the mutation load of a given tumor were identified. Such an adjustment is crucial given the higher probability of passenger mutations in driver genes in tumors with a high tumor mutation burden. A significant enrichment in activating mutations in receptor tyrosine kinase (RTK) genes were found in patients who did not derive durable clinical benefit from immune checkpoint blockade (Mann-Whitney p<0.001, FDR-adjusted p=0.002, FIG. 5, Table 5). The RTK superfamily of cell-surface receptors serve as mediators of cell signaling by extra-cellular growth factors and these oncogenes can be activated by point mutations, amplifications (FGFR1, IGF1R) or both (EGFR, ERBB2, MET). EGFR exon 19 in-frame deletions (745KELREA>T, E746_A750del, L747_T751del), exon 20 in-frame insertions (N771_H773dup) and exon 21 point mutations (L858R) as well as ERBB2 exon 19 (E770_A771insAYVM) and exon 20 (776G>VC) in-frame insertions were exclusively found in nonresponding tumors in cohort 1 (FIG. 5). Similarly, EGFR, ERBB2, MET and IGF1R amplifications were only observed in non-responding tumors, and FGFR1 amplifications were detected in 2 non-responding and one responding tumor (FIG. 5). The distribution of activating RTK mutations was independent of TMB (Mann-Whitney p=0.33) and remained significantly associated with clinical response to immune checkpoint blockade after correction for TMB (logistic regression p=0.04, Table 7). A significantly lower CD8+ T cell infiltration was found in tumors with activating RTK mutations (CD8+ T cell density of 7.36±2.5 vs. 15.16±2.5 for tumors with and without activating RTK mutations, p=0.036), indicating that RTK signaling may be linked to intratumoral T cell depletion. RTK activating mutations conferred reduced survival (log rank p=0.005, FIG. 4C) and these observations were validated in cohort 2, where an enrichment in activating mutations in RTK genes was found in non-responding tumors and resulted in worse progression-free survival (log rank p=0.009, FIG. 4D, FIG. 8). Analysis of a third independent cohort of 240 NSCLC patients treated with ICB and where tumors were analyzed with targeted NGS confirmed these findings, revealing that RTK activating mutations in EGFR, ERBB2, MET, FGFR1 and IGF1R were enriched in non-responding tumors (Fisher's exact p=0.027). RTK alterations were associated with shorter progression-free survival (log rank p=0.035; FIG. 8) independent of TMB (Mann-Whitney p=0.11 for TMB differences between responding and nonresponding tumors).

TABLE 7
Gene enrichment analysis in patients with differential responses
to immune checkpoint blockade.

| Gene/ | DCB | NDB | | FDR | TMB adjusted |
|---|---|---|---|---|---|
| Gene group | count | count | p value | p value | p value |
| RTK mutations | 1 | 15 | <0.001 | 0.002 | 0.040 |
| KRAS | 16 | 13 | 0.364 | 0.545 | 0.660 |
| STK11 | 5 | 8 | 0.559 | 0.559 | 0.153 |
| ARID1A | 9 | 1 | 0.005 | 0.024 | 0.062 |
| KEAP1 | 4 | 9 | 0.240 | 0.439 | 0.074 |
| JAK1/JAK2 | 3 | 0 | 0.101 | 0.302 | 0.391 |
| PTEN | 1 | 0 | 0.471 | 0.559 | NE |
| MDM2/MDM4 | 0 | 3 | 0.244 | 0.439 | NE |
| TP53 | 24 | 23 | 0.519 | 0.559 | 0.552 |

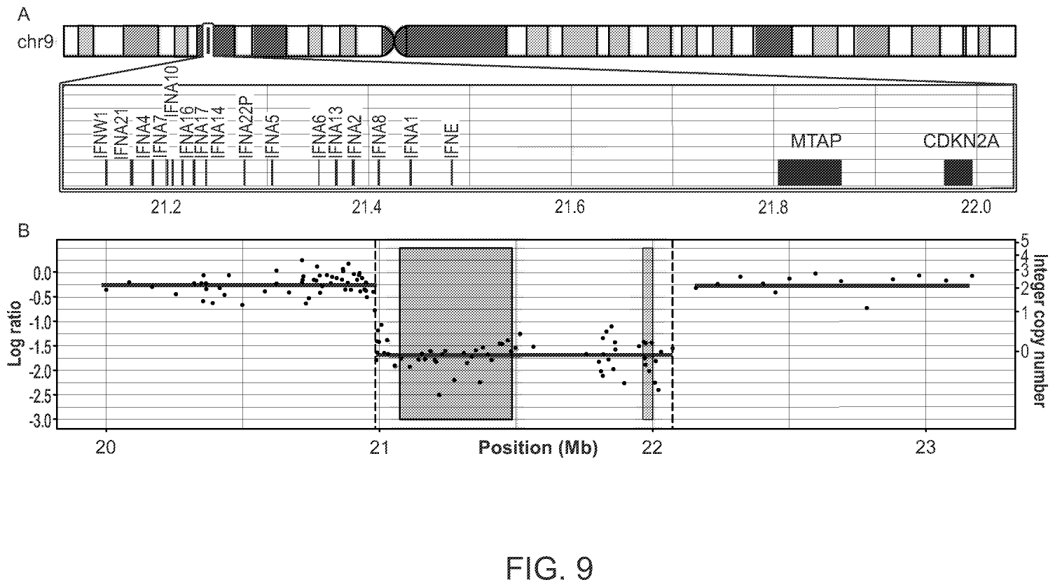

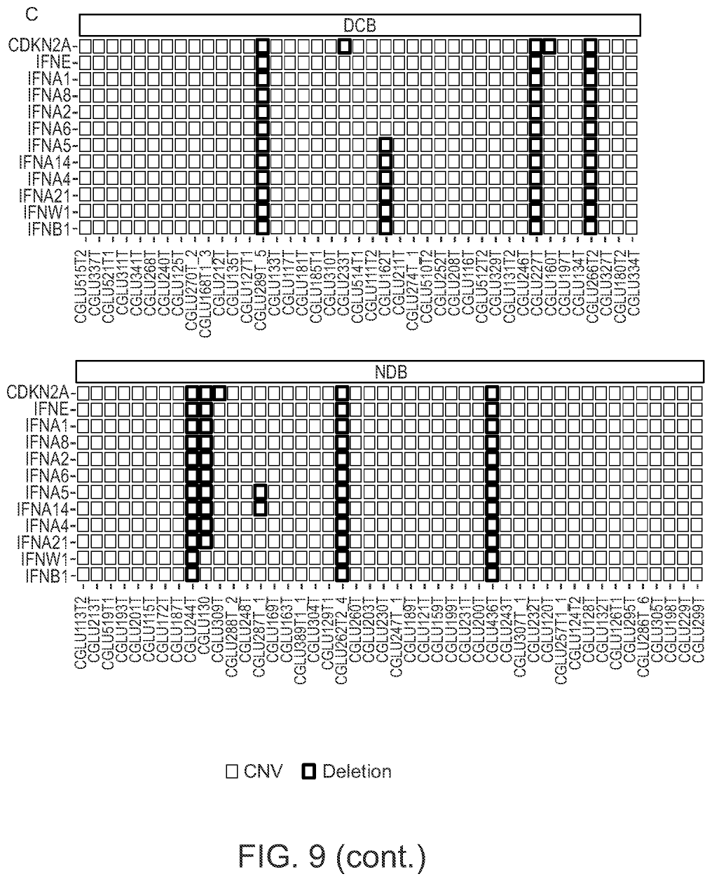

Recurrent alterations in ARID1A were found in patients with durable clinical benefit (Mann-Whitney p=0.005, FDR-adjusted p=0.024), with a trend towards statistical significance after correction for TMB (p=0.062, FIG. 5, Table 7). KEAP1 mutations, in particular inactivating mutations and loss of the wild type allele, were more commonly found in patients with non-durable clinical benefit however this observation did not reach statistical significance (p=0.074, FIG. 5, Table 7). A homozygous deletion in PTEN was found in one patient with a short-lived response to immune checkpoint blockade and MDM2/MDM4 amplifications were identified in 3 patients with non-durable clinical benefit (FIG. 5). Loss-of-function mutations were not detected in JAK1 or JAK2 nor was an enrichment of co-occurring KRAS and inactivating STK11 mutations in non-responding tumors detected (FIG. 5). Additionally, homozygous deletions were observed in IFN-γ pathway genes but their frequency was similar in responding and non-responding tumors and these deletions co-occurred with loss of the CDKN2A tumor suppressor gene in all but two of the nine cases in which they were present (FIG. 9). CDKN2A and the group of IFN-γ pathway genes are on chromosome 9p 917 Kb apart, and therefore IFN-γ deletions may be co-occurring passengers in the setting of a driver CDKN2A deletion.

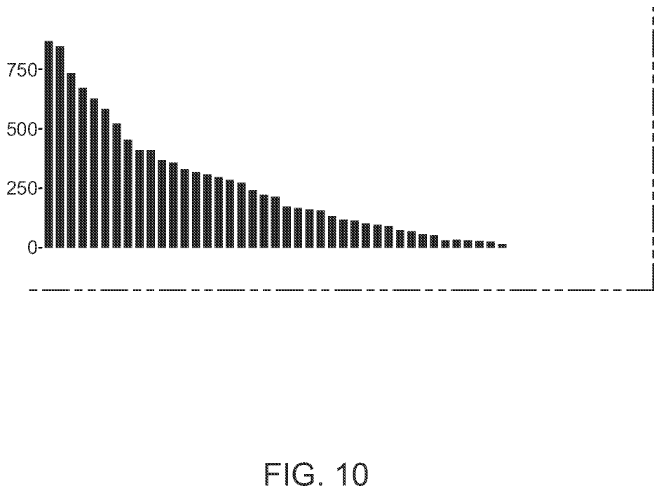

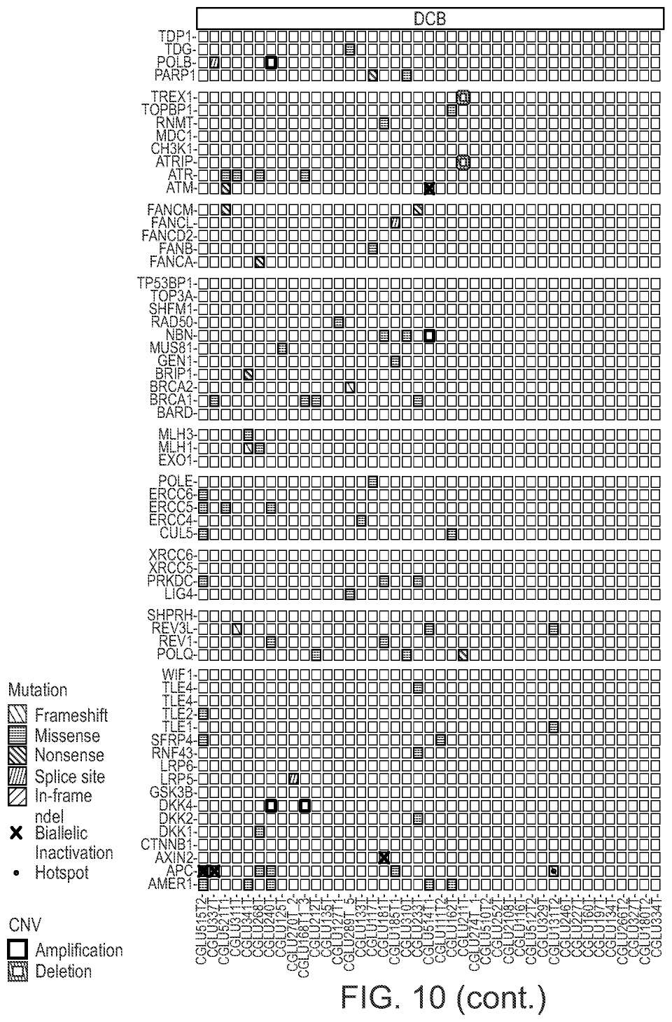

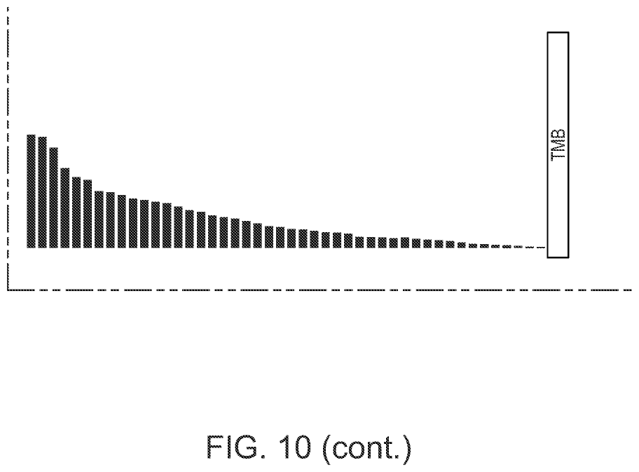

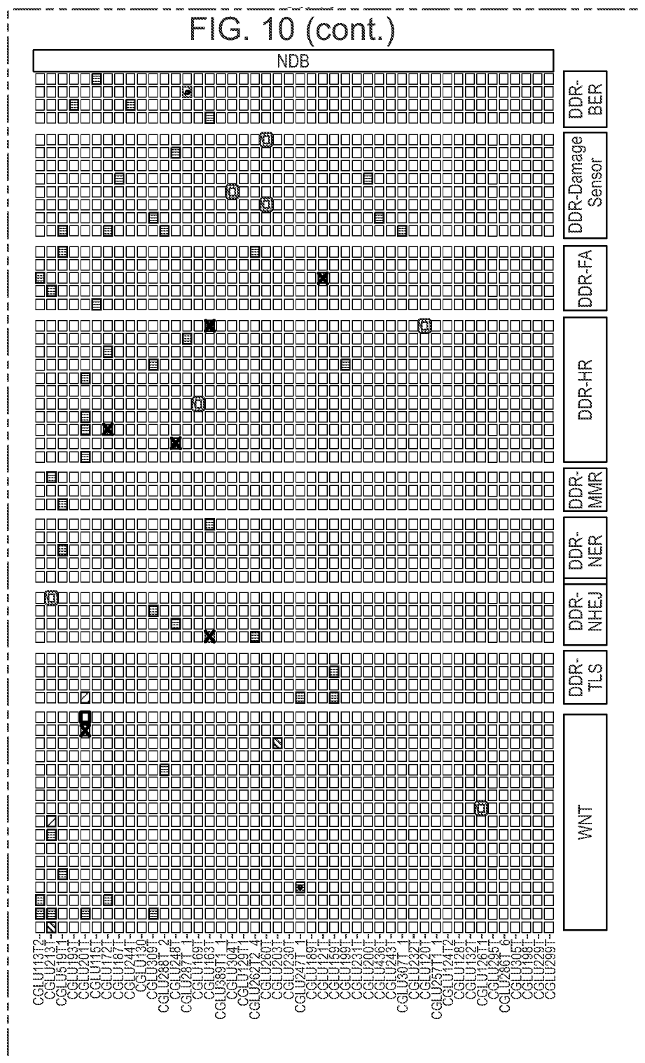

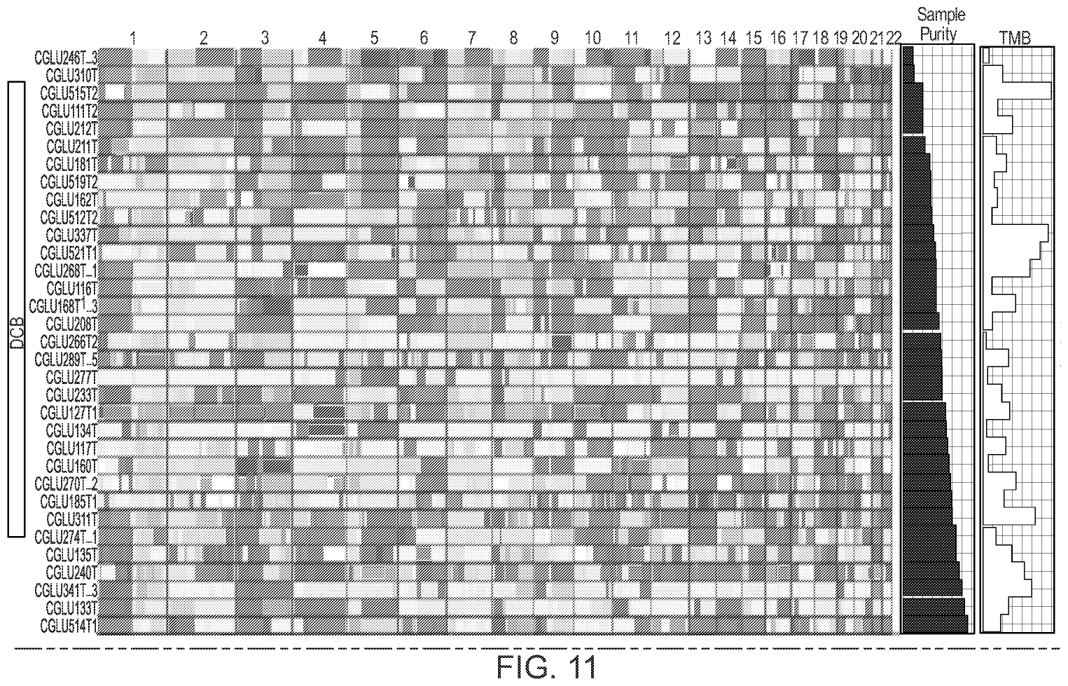

A pathway-focused approach was followed in order to identify enrichment or mutual exclusivity of genomic alterations in oncogenic processes or signaling pathways. DNA damage repair (DDR) genes and the WNT-β-catenin pathway were considered. One responding TMB-high tumor was identified with biallellic inactivation of MLH1, but an overall enrichment was not identified in deleterious somatic DDR gene mutations in responding tumors (FIG. 10). Similarly, a gain-of-function CTNNBJ hotspot mutation was detected in a non-responding tumor but no additional differences in activating mutations in the WNT pathway between responders and non-responders were detected (FIG. 10). Genome-wide copy number analyses were employed to investigate differences in tumor aneuploidy (Methods), however no significant differences were found in the fraction of the genome with allelic imbalance or LOH between patients with durable and non-durable clinical benefit (FIG. 11).

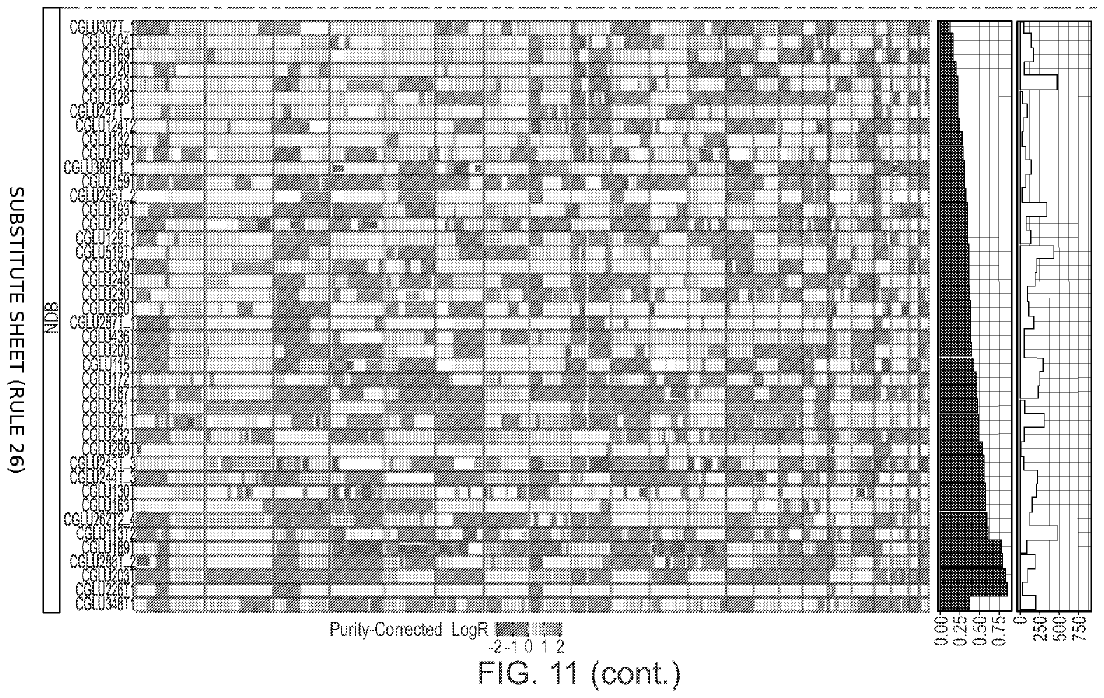

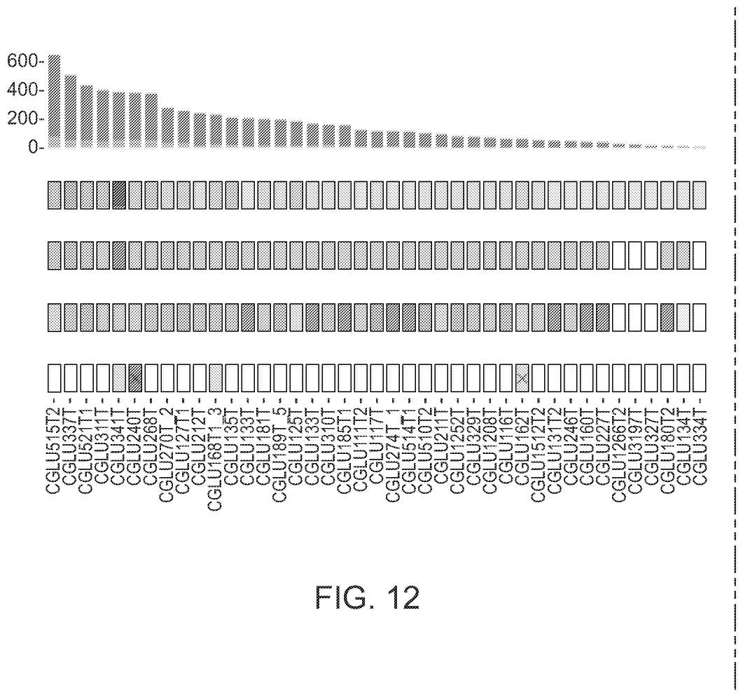

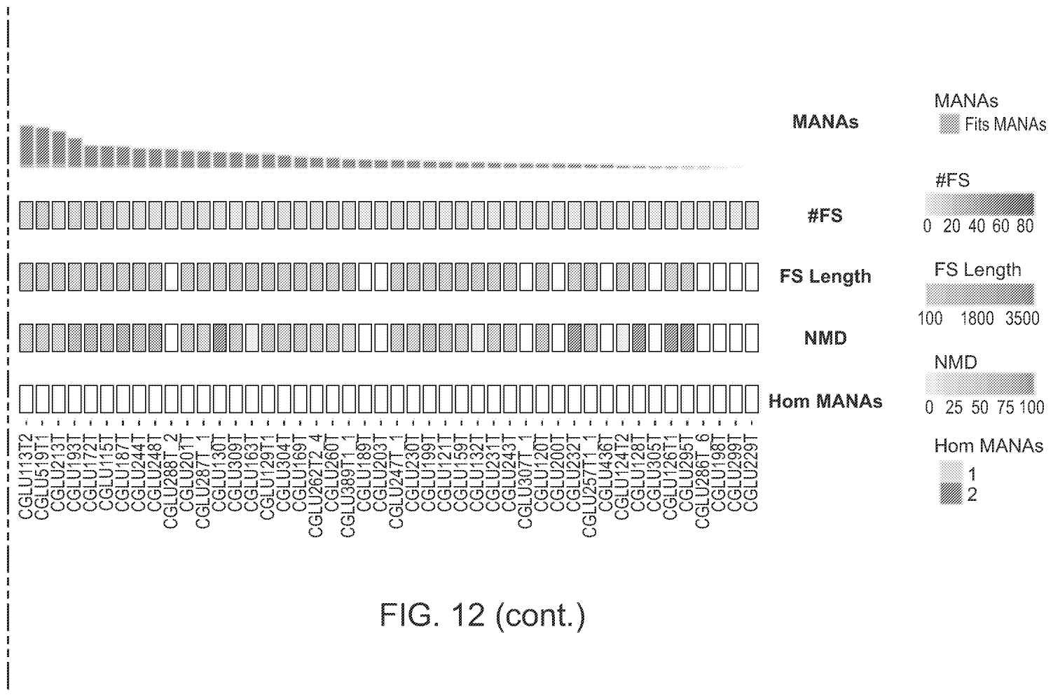

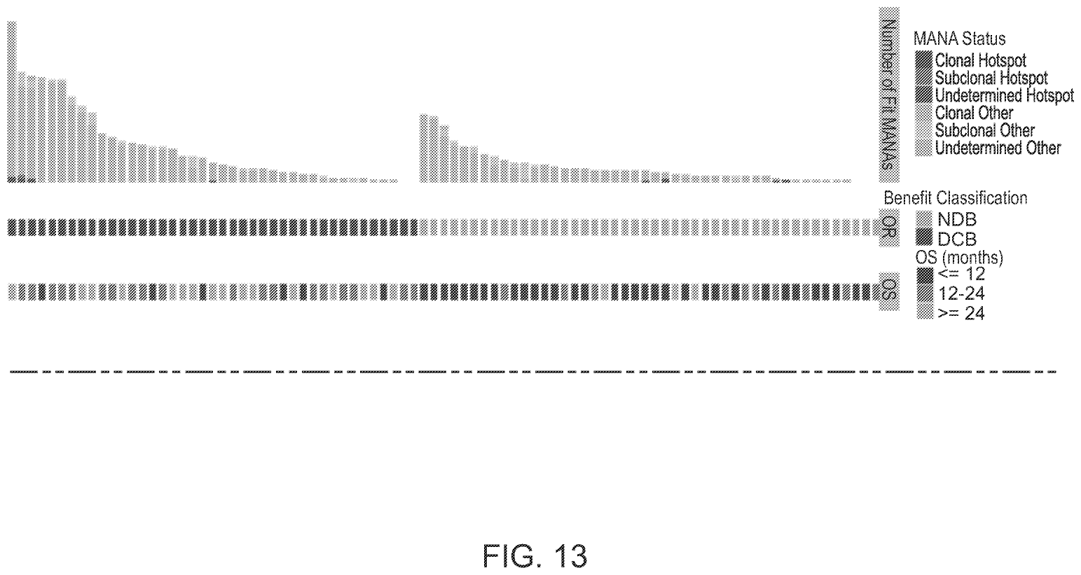

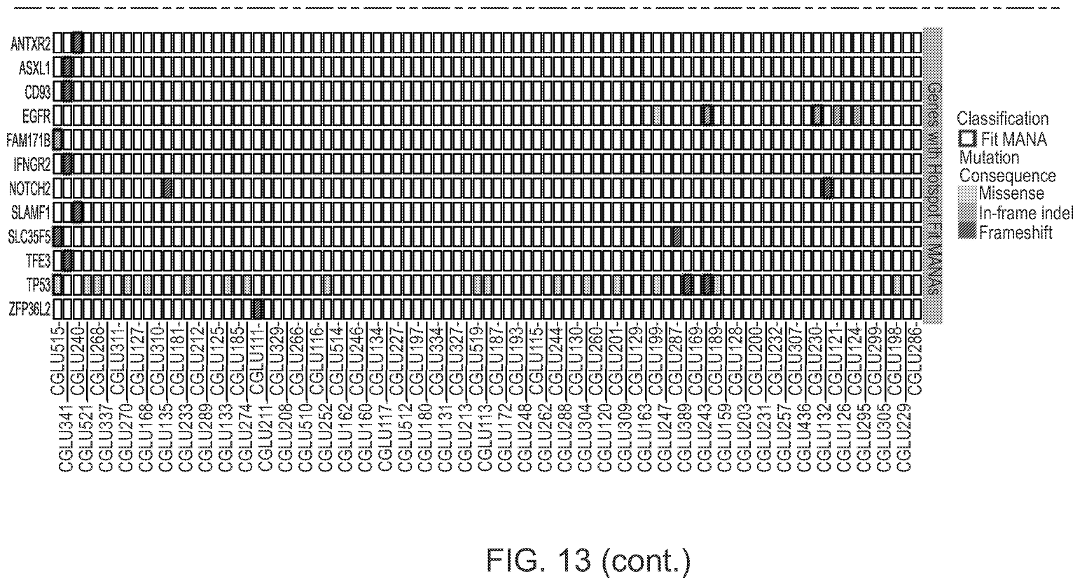

A strong correlation was found between TMB and predicted MANA load (R=0.98, p<0.001). As only a small fraction of predicted MANAs are immunogenic, neoantigens that have predicted MHC affinities ≤50 nM and for which the corresponding wild-type peptide does not bind MHC class I (affinity >1000 nM) were focused on as these “fit” neoantigens are most likely to be identified as non-self by the immune system and potentiate an anti-tumor immune response. A higher number of fit MANAs was found in responding vs. non-responding tumors (Mann-Whitney p=0.01, FDR-adjusted p=0.05; FIG. 12 and Table 5). Neoantigens stemming from frameshift alterations were further focused on, as conceptually these could generate multiple immunogenic neoantigens. Responding tumors showed a trend for a higher number of MANAs predicted to be derived from frameshift mutations (Mann-Whitney p=0.08; FIG. 12). The potential of hotspot mutations in driver and other genes to generate fit MANAs was then studied as such alterations may be less likely to be eliminated as a means of immune escape. A subset of clonal hotspot frameshifts and in-frame indels generated fit MANAs (FIG. 13) and patients harboring fit hotspot MANAs showed a trend towards longer overall survival (log rank p=0.1).

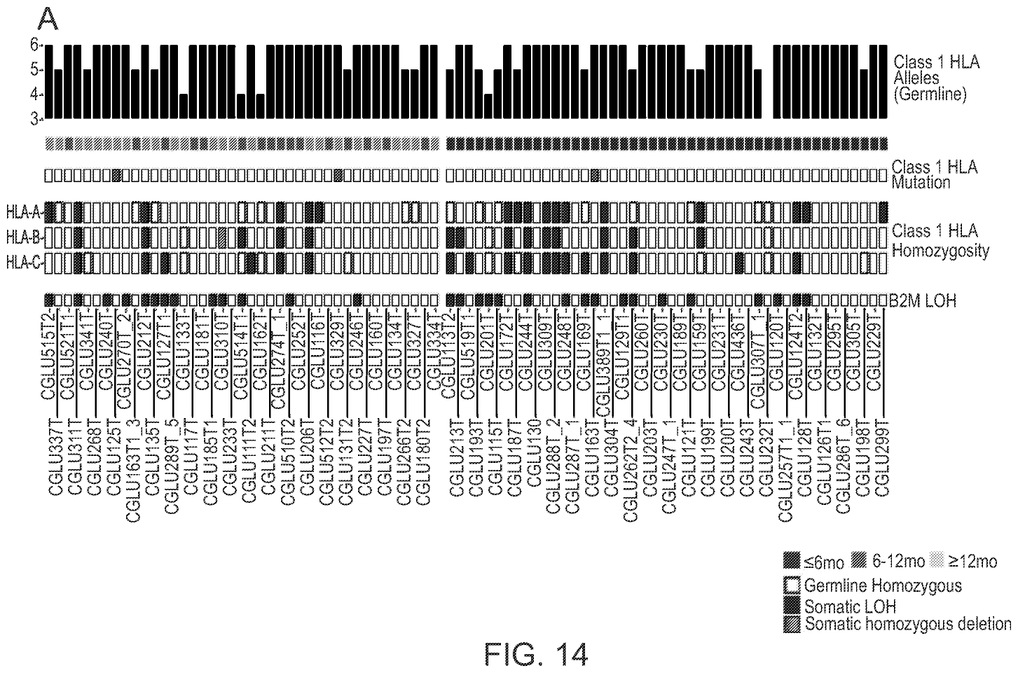

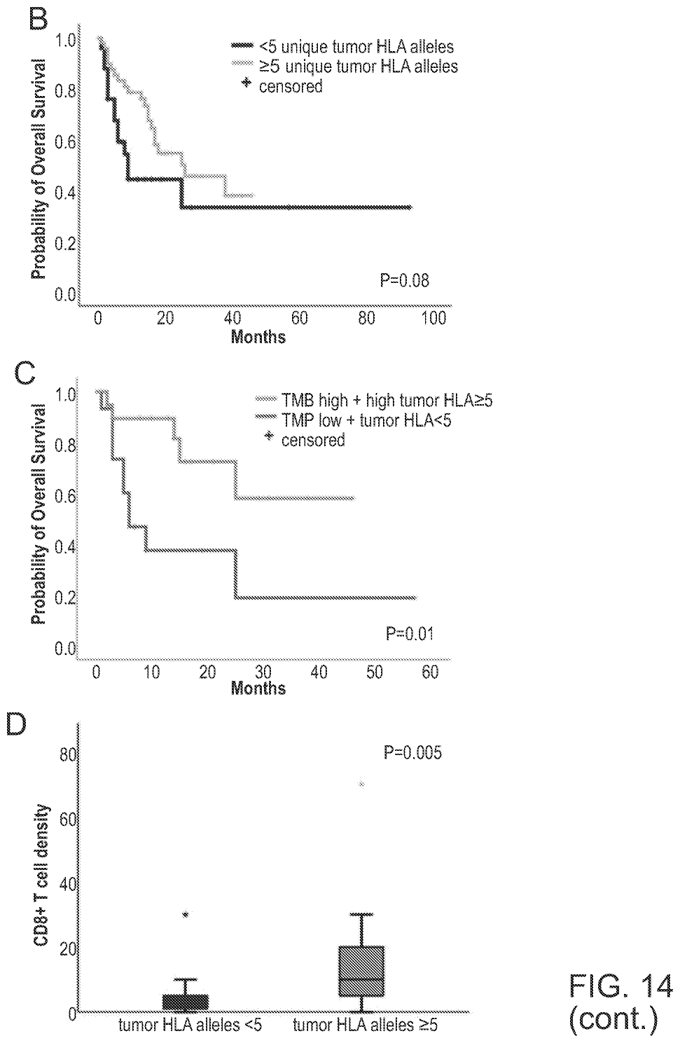

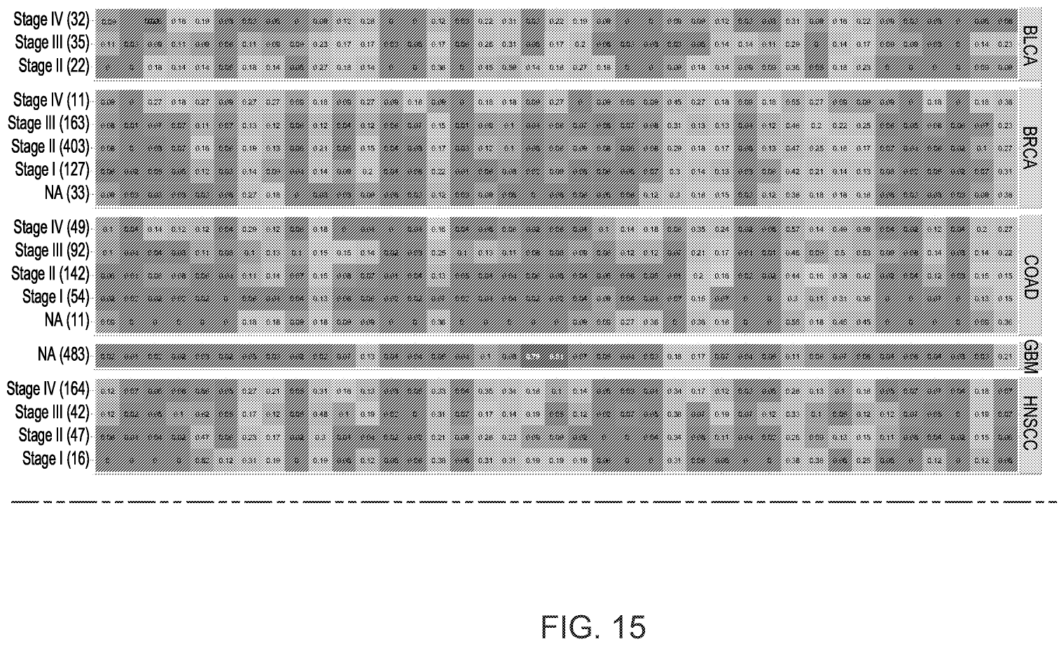

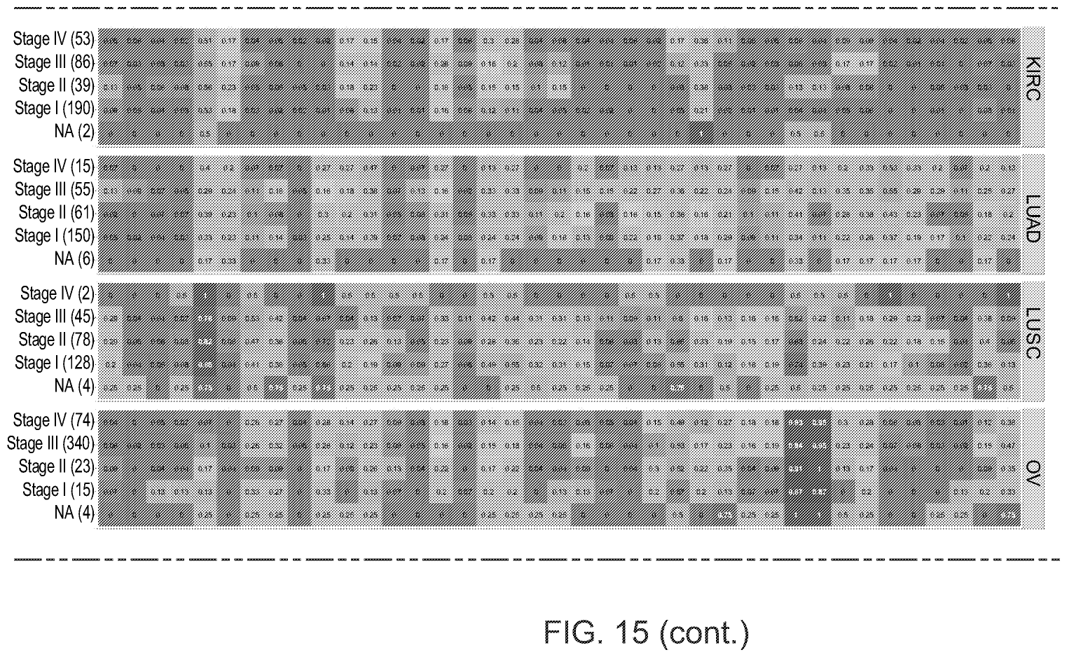

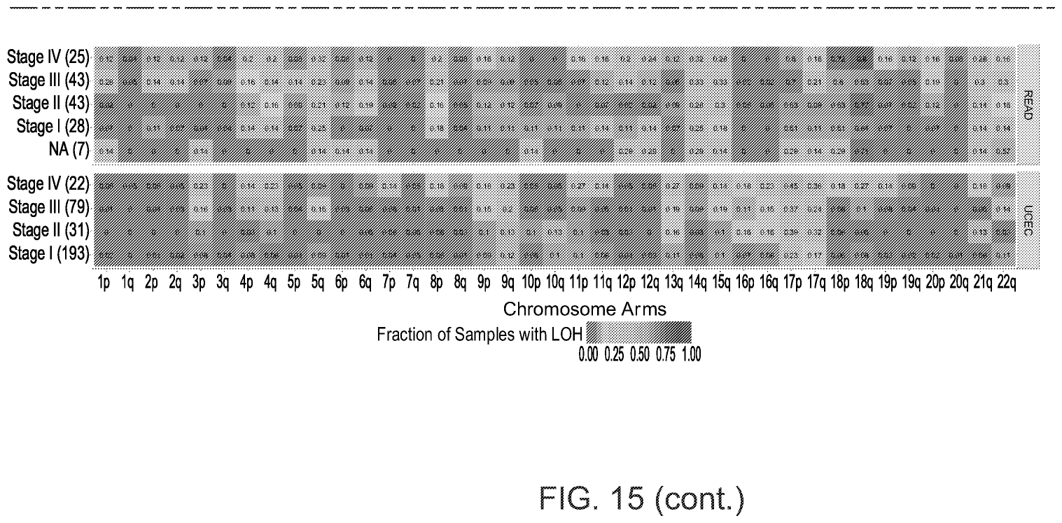

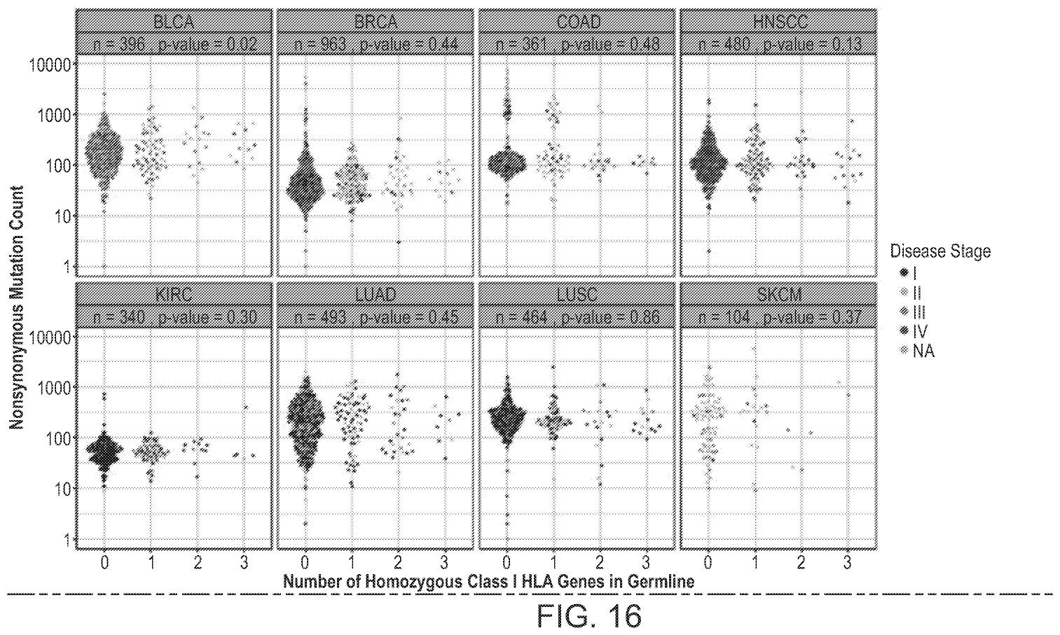

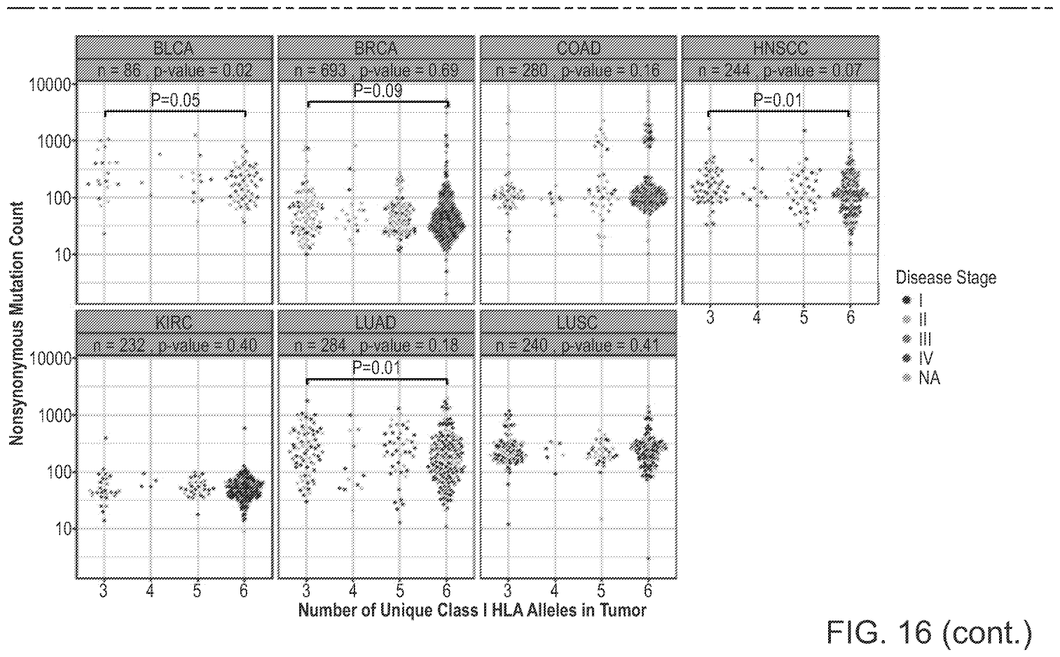

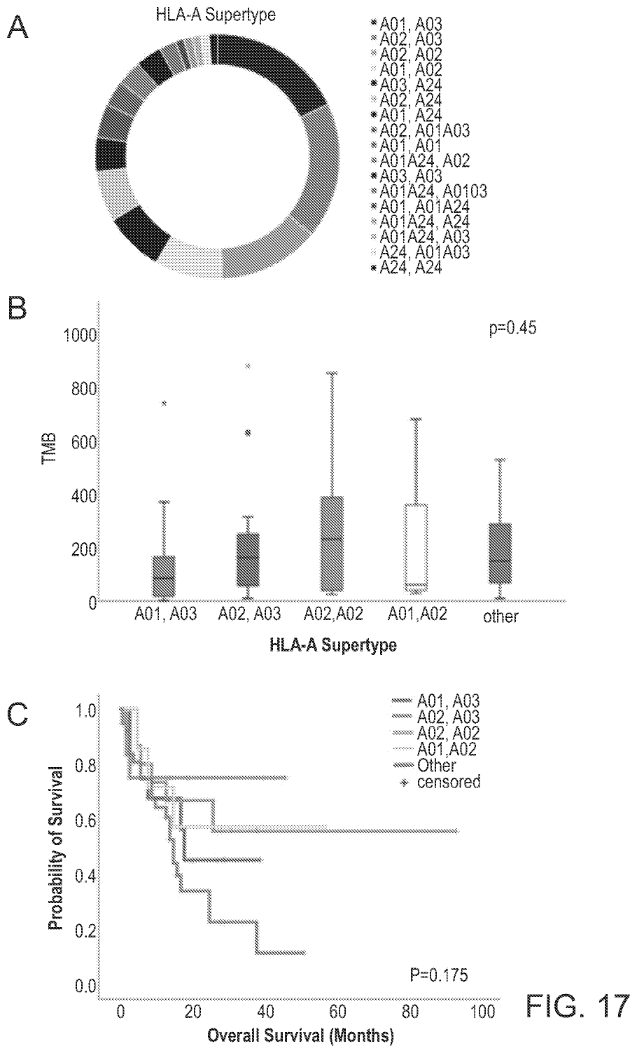

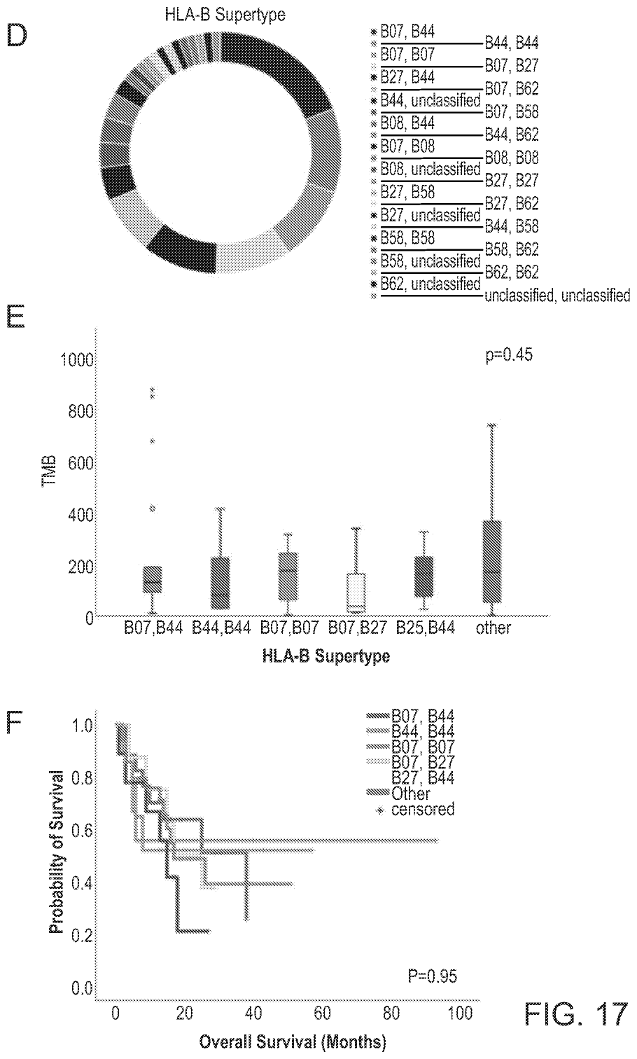

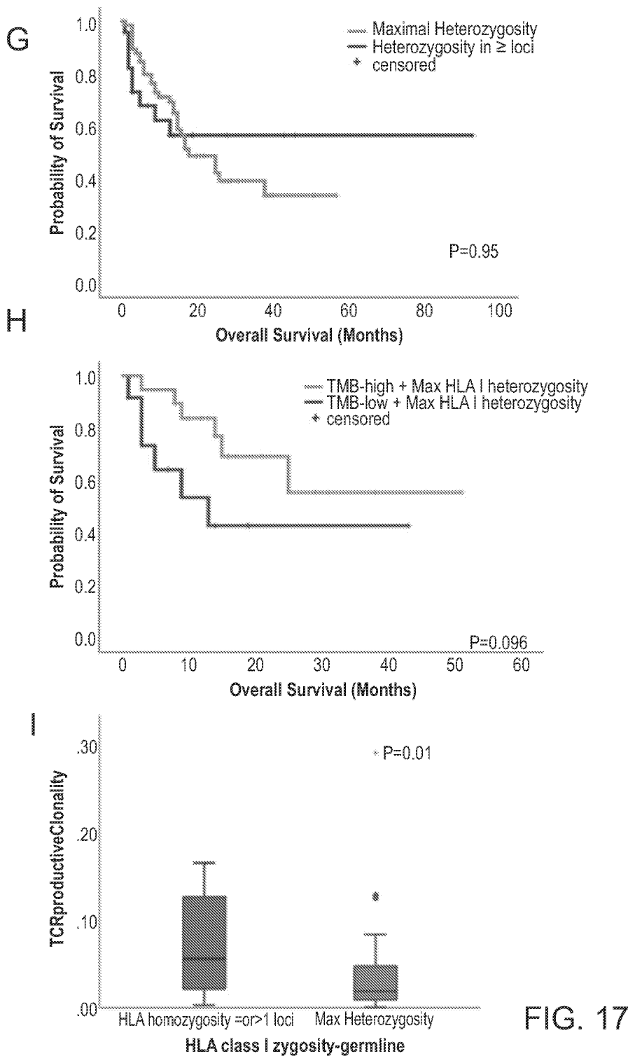

Antigen presentation deficiency may lead to immune escape through both HLA class I germline homozygosity and somatic loss of heterozygosity (LOH). In the cohort, 22 cases were homozygous for at least one HLA class I locus in their germline, and somatic HLA LOH occurred in 27 tumors (FIG. 14A and Table 11). Mutations in HLA class I genes were rare (only seen in 3 cases). Through analysis of 3,601 TCGA samples, no enrichment was found in LOH of chromosome 6p that contains the HLA class I loci compared to background arm-level allelic imbalance in NSCLC, but the degree of 6p LOH was higher in NSCLC compared to other tumor types (p<0.001, FIG. 15). The β2-microglobulin locus was frequently lost by LOH, however concurrent inactivating mutations were detected, rendering this an infrequent mechanism of immune evasion in our cohort (FIG. 14A). Conceptually, tumors with increased mutation burden would be more likely to be recognized by the immune system but may overcome this evolutionary disadvantage through HLA haplotype loss and diminished presentation potential of neoantigens. While germline HLA zygosity was not correlated with TMB for the vast majority of tumors examined, combined germline and tumor HLA status was correlated with TMB such that tumors with a lower non-synonymous mutation load harbored a more intact antigen presentation capacity (FIG. 16). No association was found between TMB and HLA class I supertypes, and germline HLA class I variation was not associated with outcome (FIG. 17). HLA class I germline zygosity and somatic HLA class I LOH events were combined to determine the effect of unique number of HLA class I alleles on response to ICB. Tumors with reduced antigen presentation potential were linked to worse outcome to ICB (FIG. 14B) and importantly, when antigen presentation capacity and TMB were combined, NSCLCs with low TMB and reduced antigen presentation potential had a significantly shorter overall survival (log rank p=0.01, FIG. 14C). Tumors with lower antigen presentation capacity showed a significantly lower level of CD8+ T cell density (Mann Whitney p=0.005, FIG. 14D), suggesting that these tumors may present a less diverse neoantigen repertoire to cytotoxic T cells and therefore have the potential to become partially invisible to the immune system. Furthermore, cases with maximal HLA class I heterozygosity were found to have a less clonal TCR repertoire (p=0.01, FIG. 17), suggesting that HLA variation determines the selection and clonal expansion of neoantigen-specific T cells.

TABLE 11
HLA class I genomic variation

| | HLA-A | HLA-A | HLA-B | HLA-B | HLA-C | HLA-C | | HLA-A | HLA-B | HLA-C |
|---|---|---|---|---|---|---|---|---|---|---|
| Patient ID | Allele 1 | Allele 2 | Allele 1 | Allele 2 | Allele 1 | Allele 2 | HLA mutation | LOH | LOH | LOH |
| CGLU111 | HLA-A02:01 | HLA-A02:02 | HLA-B07:02 | HLA-B42:01 | HLA-C07:02 | HLA-C17:01 | — | NA | FALSE | TRUE |
| CGLU113 | HLA-A02:01 | HLA-A02:01 | HLA-B07:02 | HLA-B57:01 | HLA-C06:02 | HLA-C07:02 | — | NA | TRUE | TRUE |
| CGLU115 | HLA-A02:01 | HLA-A02:01 | HLA-B08:01 | HLA-B78:01 | HLA-C07:01 | HLA-C16:01 | — | NA | FALSE | FALSE |
| CGLU116 | HLA-A01:01 | HLA-A31:01 | HLA-B51:02 | HLA-B52:01 | HLA-C12:02 | HLA-C15:02 | — | TRUE | FALSE | FALSE |
| CGLU117 | HLA-A24:02 | HLA-A26:01 | HLA-B08:01 | HLA-B35:02 | HLA-C04:01 | HLA-C07:02 | — | FALSE | FALSE | FALSE |
| CGLU120 | HLA-A29:02 | HLA-A30:01 | HLA-B15:03 | HLA-B42:01 | HLA-C02:10 | HLA-C17:01 | — | FALSE | FALSE | FALSE |
| CGLU121 | HLA-A03:01 | HLA-A03:01 | HLA-B07:02 | HLA-B27:05 | HLA-C02:02 | HLA-C07:02 | — | NA | FALSE | FALSE |
| CGLU124 | HLA-A02:01 | HLA-A24:02 | HLA-B40:02 | HLA-B41:01 | HLA-C02:02 | HLA-C17:01 | — | TRUE | FALSE | TRUE |
| CGLU125 | HLA-A24:02 | HLA-A25:01 | HLA-B57:01 | HLA-B58:01 | HLA-C06:02 | HLA-C07:01 | HLA-A24:02 | NA | NA | NA |
| | | | | | | | (p.A64fs) | | | |
| CGLU126 | HLA-A29:02 | HLA-A30:01 | HLA-B13:02 | HLA-B44:03 | HLA-C06:02 | HLA-C16:01 | — | FALSE | FALSE | FALSE |
| CGLU127 | HLA-A02:01 | HLA-A30:01 | HLA-B39:01 | HLA-B42:01 | HLA-C07:02 | HLA-C17:01 | — | FALSE | FALSE | TRUE |
| CGLU128 | HLA-A03:01 | HLA-A24:02 | HLA-B44:02 | HLA-B55:01 | HLA-C03:03 | HLA-C05:01 | — | TRUE | FALSE | FALSE |
| CGLU129 | HLA-A02:01 | HLA-A03:01 | HLA-B08:01 | HLA-B44:03 | HLA-C07:01 | HLA-C16:01 | — | FALSE | FALSE | FALSE |
| CGLU130 | HLA-A02:01 | HLA-A23:01 | HLA-B44:02 | HLA-B49:01 | HLA-C07:01 | HLA-C07:04 | — | FALSE | FALSE | FALSE |
| CGLU131 | HLA-A25:01 | HLA-A26:14 | HLA-B18:01 | HLA-B38:01 | HLA-C12:03 | HLA-C12:03 | — | NA | NA | NA |
| CGLU132 | HLA-A23:01 | HLA-A68:02 | HLA-B07:02 | HLA-B42:01 | HLA-C07:02 | HLA-C17:01 | — | FALSE | FALSE | FALSE |
| CGLU133 | HLA-A02:01 | HLA-A03:01 | HLA-B07:02 | HLA-B07:02 | HLA-C07:02 | HLA-C07:02 | — | FALSE | NA | NA |
| CGLU134 | HLA-A01:01 | HLA-A02:01 | HLA-B44:02 | HLA-B44:03 | HLA-C04:01 | HLA-C05:01 | — | TRUE | FALSE | TRUE |
| CGLU135 | HLA-A02:01 | HLA-A02:01 | HLA-B44:02 | HLA-B49:01 | HLA-C05:01 | HLA-C07:01 | — | NA | FALSE | FALSE |
| CGLU159 | HLA-A01:01 | HLA-A02:05 | HLA-B08:01 | HLA-B49:01 | HLA-C07:01 | HLA-C07:01 | — | TRUE | TRUE | NA |
| CGLU160 | HLA-A02:01 | HLA-A03:01 | HLA-B07:02 | HLA-B15:01 | HLA-C03:04 | HLA-C07:02 | — | FALSE | FALSE | FALSE |
| CGLU162 | HLA-A01:01 | HLA-A01:01 | HLA-B08:01 | HLA-B18:01 | HLA-C07:01 | HLA-C07:01 | — | NA | FALSE | NA |
| CGLU163 | HLA-A03:01 | HLA-A26:01 | HLA-B15:01 | HLA-B45:01 | HLA-C03:03 | HLA-C06:02 | HLA-C03:03 | FALSE | FALSE | FALSE |
| | | | | | | | (p.V271M) | | | |
| CGLU168 | HLA-A02:01 | HLA-A02:01 | HLA-B44:02 | HLA-B44:03 | HLA-C04:01 | HLA-C05:01 | — | NA | NA | FALSE |
| CGLU169 | HLA-A24:02 | HLA-A24:02 | HLA-B39:06 | HLA-B41:02 | HLA-C07:02 | HLA-C17:01 | — | NA | FALSE | TRUE |
| CGLU172 | HLA-A23:01 | HLA-A66:03 | HLA-B15:03 | HLA-B44:03 | HLA-C02:10 | HLA-C04:01 | — | TRUE | TRUE | TRUE |
| CGLU178 | HLA-A01:03 | HLA-A29:01 | HLA-B07:05 | HLA-B73:01 | HLA-C15:05 | HLA-C15:05 | — | NA | NA | NA |
| CGLU180 | HLA-A01:01 | HLA-A33:01 | HLA-B14:02 | HLA-B52:01 | HLA-C08:02 | HLA-C12:02 | — | NA | NA | NA |
| CGLU181 | HLA-A23:01 | HLA-A30:01 | HLA-B07:02 | HLA-B42:01 | HLA-C07:02 | HLA-C17:01 | — | FALSE | FALSE | FALSE |
| CGLU185 | HLA-A02:01 | HLA-A66:03 | HLA-B15:01 | HLA-B44:03 | HLA-C01:02 | HLA-C04:01 | — | FALSE | FALSE | FALSE |
| CGLU187 | HLA-A02:01 | HLA-A30:01 | HLA-B13:02 | HLA-B57:01 | HLA-C06:02 | HLA-C06:02 | — | TRUE | FALSE | NA |
| CGLU189 | HLA-A02:01 | HLA-A31:01 | HLA-B07:02 | HLA-B15:01 | HLA-C03:04 | HLA-C07:02 | — | FALSE | FALSE | FALSE |
| CGLU193 | HLA-A02:01 | HLA-A02:01 | HLA-B44:02 | HLA-B45:01 | HLA-C05:01 | HLA-C06:02 | — | NA | FALSE | FALSE |
| CGLU197 | HLA-A02:01 | HLA-A03:01 | HLA-B07:02 | HLA-B41:02 | HLA-C07:02 | HLA-C17:01 | — | NA | NA | NA |
| CGLU198 | HLA-A32:01 | HLA-A33:03 | HLA-B15:17 | HLA-B44:03 | HLA-C07:01 | HLA-C07:01 | — | NA | NA | NA |
| CGLU199 | HLA-A02:07 | HLA-A11:01 | HLA-B15:01 | HLA-B46:01 | HLA-C01:02 | HLA-C12:02 | — | TRUE | FALSE | TRUE |
| CGLU200 | HLA-A03:01 | HLA-A68:01 | HLA-B07:02 | HLA-B35:03 | HLA-C04:01 | HLA-C07:02 | — | FALSE | FALSE | FALSE |
| CGLU201 | HLA-A01:01 | HLA-A24:02 | HLA-B08:01 | HLA-B08:01 | HLA-C07:01 | HLA-C07:01 | — | TRUE | NA | NA |
| CGLU203 | HLA-A01:01 | HLA-A24:02 | HLA-B37:01 | HLA-B40:01 | HLA-C03:04 | HLA-C06:02 | — | FALSE | FALSE | FALSE |
| CGLU208 | HLA-A02:01 | HLA-A29:02 | HLA-B35:03 | HLA-B44:03 | HLA-C04:01 | HLA-C16:01 | — | TRUE | TRUE | TRUE |
| CGLU211 | HLA-A68:02 | HLA-A74:01 | HLA-B07:05 | HLA-B15:10 | HLA-C03:04 | HLA-C15:05 | — | FALSE | FALSE | FALSE |
| CGLU212 | HLA-A03:01 | HLA-A25:01 | HLA-B15:01 | HLA-B44:02 | HLA-C03:03 | HLA-C05:01 | — | TRUE | TRUE | TRUE |
| CGLU213 | HLA-A02:01 | HLA-A29:02 | HLA-B14:02 | HLA-B57:01 | HLA-C06:02 | HLA-C08:02 | — | FALSE | TRUE | FALSE |
| CGLU227 | HLA-A02:05 | HLA-A30:02 | HLA-B27:05 | HLA-B49:01 | HLA-C02:02 | HLA-C07:01 | — | FALSE | FALSE | FALSE |
| CGLU229 | HLA-A26:01 | HLA-A31:01 | HLA-B07:02 | HLA-B51:01 | HLA-C07:02 | HLA-C14:02 | — | NA | NA | NA |
| CGLU230 | HLA-A11:01 | HLA-A24:02 | HLA-B15:21 | HLA-B38:02 | HLA-C04:03 | HLA-C07:27 | — | FALSE | FALSE | FALSE |
| CGLU231 | HLA-A26:01 | HLA-A68:01 | HLA-B38:01 | HLA-B44:02 | HLA-C07:04 | HLA-C12:03 | — | FALSE | FALSE | FALSE |
| CGLU232 | HLA-A02:01 | HLA-A02:01 | HLA-B44:02 | HLA-B44:02 | HLA-C05:01 | HLA-C05:01 | — | NA | NA | NA |
| CGLU233 | HLA-A02:11 | HLA-A33:03 | HLA-B15:18 | HLA-B40:06 | HLA-C07:04 | HLA-C15:02 | — | FALSE | FALSE | FALSE |
| CGLU240 | HLA-A11:03 | HLA-A24:02 | HLA-B35:01 | HLA-B52:01 | HLA-C03:03 | HLA-C07:02 | — | FALSE | FALSE | FALSE |
| CGLU243 | HLA-A02:07 | HLA-A33:03 | HLA-B46:01 | HLA-B58:01 | HLA-C01:02 | HLA-C03:02 | — | FALSE | FALSE | FALSE |
| CGLU244 | HLA-A25:01 | HLA-A33:03 | HLA-B18:01 | HLA-B44:02 | HLA-C07:04 | HLA-C12:03 | — | TRUE | TRUE | TRUE |
| CGLU246 | HLA-A03:01 | HLA-A32:01 | HLA-B18:01 | HLA-B40:01 | HLA-C03:04 | HLA-C07:01 | — | FALSE | FALSE | FALSE |
| CGLU247 | HLA-A01:01 | HLA-A31:01 | HLA-B07:02 | HLA-B40:01 | HLA-C03:04 | HLA-C07:02 | — | FALSE | FALSE | FALSE |
| CGLU248 | HLA-A02:01 | HLA-A03:01 | HLA-B07:02 | HLA-B37:01 | HLA-C06:02 | HLA-C07:02 | — | TRUE | FALSE | TRUE |
| CGLU252 | HLA-A03:01 | HLA-A23:01 | HLA-B07:02 | HLA-B44:03 | HLA-C04:01 | HLA-C07:02 | — | NA | NA | NA |
| CGLU257 | HLA-A01:01 | HLA-A02:01 | HLA-B07:02 | HLA-B14:02 | HLA-C07:02 | HLA-C08:02 | — | NA | NA | NA |
| CGLU260 | HLA-A02:01 | HLA-A29:02 | HLA-B44:02 | HLA-B44:03 | HLA-C05:01 | HLA-C16:01 | — | FALSE | NA | FALSE |
| CGLU262 | HLA-A02:01 | HLA-A02:01 | HLA-B40:01 | HLA-B51:01 | HLA-C03:04 | HLA-C05:01 | — | NA | TRUE | TRUE |
| CGLU266 | HLA-A02:01 | HLA-A02:01 | HLA-B40:01 | HLA-B44:02 | HLA-C03:04 | HLA-C05:01 | — | NA | FALSE | FALSE |
| CGLU268 | HLA-A01:01 | HLA-A02:01 | HLA-B07:02 | HLA-B57:01 | HLA-C06:02 | HLA-C07:02 | — | FALSE | FALSE | FALSE |
| CGLU270 | HLA-A02:01 | HLA-A24:02 | HLA-B07:02 | HLA-B40:01 | HLA-C03:04 | HLA-C07:02 | — | FALSE | FALSE | FALSE |
| CGLU274 | HLA-A26:01 | HLA-A68:01 | HLA-B07:02 | HLA-B51:01 | HLA-C07:02 | HLA-C15:06 | — | TRUE | TRUE | TRUE |
| CGLU286 | HLA-A01:01 | HLA-A32:01 | HLA-B07:02 | HLA-B18:01 | HLA-C07:01 | HLA-C07:02 | — | NA | NA | NA |
| CGLU287 | HLA-A01:01 | HLA-A03:01 | HLA-B07:02 | HLA-B44:02 | HLA-C05:01 | HLA-C07:02 | — | FALSE | FALSE | FALSE |
| CGLU288 | HLA-A02:01 | HLA-A03:01 | HLA-B07:02 | HLA-B51:01 | HLA-C02:02 | HLA-C07:02 | — | TRUE | TRUE | TRUE |
| CGLU289 | HLA-A02:01 | HLA-A24:02 | HLA-B27:05 | HLA-B44:02 | HLA-C01:02 | HLA-C05:01 | — | FALSE | FALSE | FALSE |
| CGLU295 | HLA-A24:02 | HLA-A29:02 | HLA-B07:02 | HLA-B38:01 | HLA-C07:02 | HLA-C12:03 | — | FALSE | FALSE | FALSE |
| CGLU299 | HLA-A30:02 | HLA-A68:01 | HLA-B15:03 | HLA-B27:03 | HLA-C02:10 | HLA-C07:02 | — | TRUE | FALSE | FALSE |
| CGLU304 | HLA-A30:02 | HLA-A68:02 | HLA-B07:02 | HLA-B18:01 | HLA-C05:01 | HLA-C15:05 | — | FALSE | FALSE | FALSE |
| CGLU305 | HLA-A02:01 | HLA-A03:01 | HLA-B07:02 | HLA-B15:01 | HLA-C03:03 | HLA-C07:02 | — | NA | NA | NA |
| CGLU307 | HLA-A02:01 | HLA-A02:01 | HLA-B07:02 | HLA-B15:01 | HLA-C03:03 | HLA-C07:02 | — | NA | FALSE | FALSE |
| CGLU309 | HLA-A03:01 | HLA-A29:02 | HLA-B14:02 | HLA-B41:01 | HLA-C08:02 | HLA-C17:01 | — | TRUE | TRUE | TRUE |
| CGLU310 | HLA-A02:01 | HLA-A23:01 | HLA-B35:01 | HLA-B53:01 | HLA-C04:01 | HLA-C16:01 | — | FALSE | FALSE | FALSE |
| CGLU311 | HLA-A02:01 | HLA-A36:01 | HLA-B45:01 | HLA-B53:01 | HLA-C04:01 | HLA-C16:01 | — | TRUE | TRUE | TRUE |
| CGLU327 | HLA-A02:01 | HLA-A02:01 | HLA-B40:01 | HLA-B48:01 | HLA-C04:01 | HLA-C08:01 | — | NA | NA | NA |
| CGLU329 | HLA-A02:01 | HLA-A31:01 | HLA-B07:02 | HLA-B40:01 | HLA-C05:01 | HLA-C07:02 | HLA-A02:01 | NA | NA | NA |
| | | | | | | | (p.T323A) | | | |
| CGLU334 | HLA-A03:01 | HLA-A25:01 | HLA-B39:01 | HLA-B51:01 | HLA-C03:03 | HLA-C12:03 | — | NA | NA | NA |
| CGLU337 | HLA-A02:01 | HLA-A02:01 | HLA-B35:03 | HLA-B44:02 | HLA-C04:01 | HLA-C16:04 | — | NA | FALSE | FALSE |
| CGLU341 | HLA-A02:01 | HLA-A03:01 | HLA-B18:01 | HLA-B49:01 | HLA-C07:01 | HLA-C07:01 | — | FALSE | FALSE | NA |
| CGLU348 | HLA-A02:01 | HLA-A30:01 | HLA-B15:01 | HLA-B53:01 | HLA-C01:02 | HLA-C04:01 | — | TRUE | FALSE | TRUE |
| CGLU389 | HLA-A23:01 | HLA-A66:03 | HLA-B44:03 | HLA-B81:01 | HLA-C04:01 | HLA-C18:01 | — | TRUE | TRUE | TRUE |
| CGLU436 | HLA-A01:01 | HLA-A02:01 | HLA-B08:01 | HLA-B44:02 | HLA-C05:01 | HLA-C07:01 | — | FALSE | FALSE | TRUE |
| CGLU510 | HLA-A02:05 | HLA-A30:01 | HLA-B50:01 | HLA-B51:01 | HLA-C06:02 | HLA-C15:02 | — | FALSE | FALSE | FALSE |
| CGLU512 | HLA-A03:01 | HLA-A26:01 | HLA-B18:01 | HLA-B38:01 | HLA-C05:01 | HLA-C12:03 | — | FALSE | FALSE | FALSE |
| CGLU514 | HLA-A03:01 | HLA-A03:01 | HLA-B38:01 | HLA-B51:01 | HLA-C12:03 | HLA-C12:03 | — | NA | TRUE | NA |
| CGLU515 | HLA-A02:01 | HLA-A11:01 | HLA-B07:02 | HLA-B44:03 | HLA-C07:02 | HLA-C16:01 | — | TRUE | FALSE | FALSE |
| CGLU519 | HLA-A24:02 | HLA-A68:01 | HLA-B13:01 | HLA-B15:25 | HLA-C04:03 | HLA-C07:01 | — | FALSE | FALSE | TRUE |
| CGLU521 | HLA-A01:01 | HLA-A03:01 | HLA-B07:02 | HLA-B57:01 | HLA-C06:02 | HLA-C07:02 | — | FALSE | FALSE | FALSE |

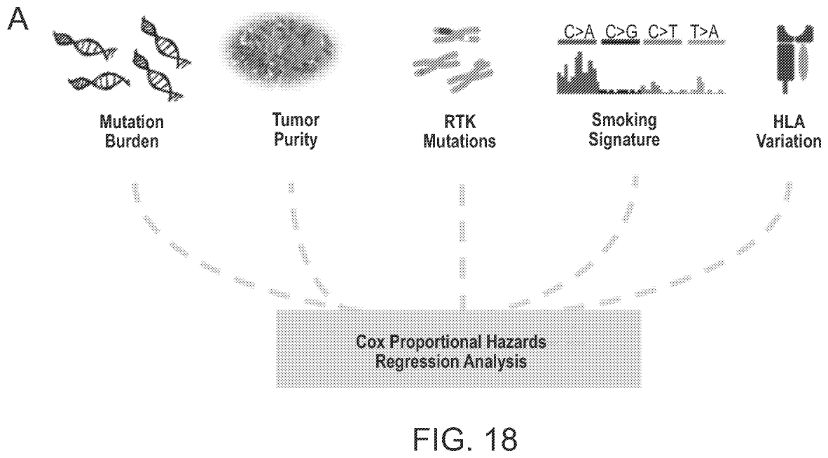

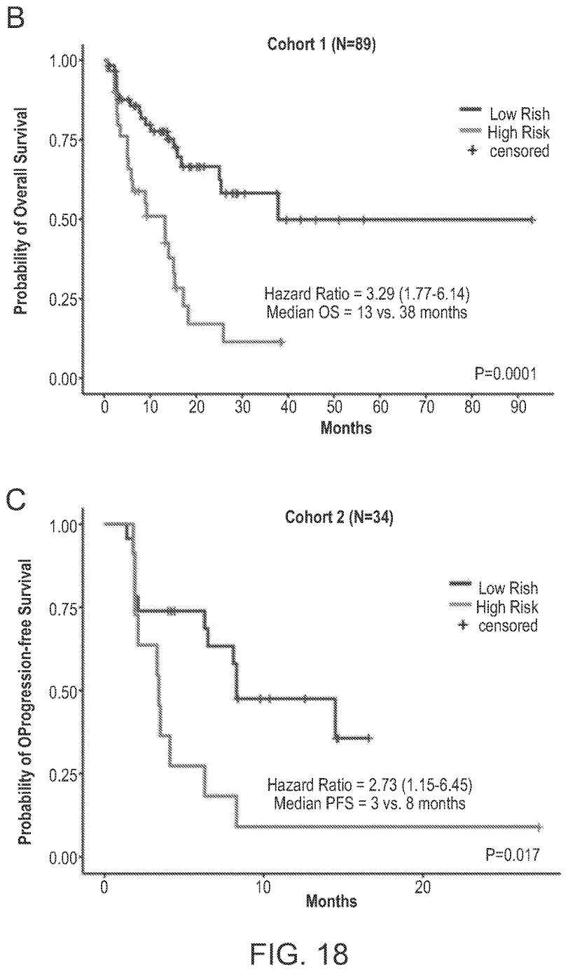

Given the importance of specific individual features identified, cTMB, molecular smoking signature, RTK activating mutations, and HLA genetic variation were combined in a multi-parameter predictor of outcome (FIG. 18A). Multivariate Cox proportional hazards regression analysis was applied to evaluate the combined contribution of these molecular features in predicting overall survival in our cohort, followed by independent validation of the model in cohort 2 (Table 12). A risk score was calculated as the exponential of the sum of product of mean-centered covariate values and their corresponding coefficient estimates and used to classify patients in high and low risk groups (Methods). Patients classified in the high risk category had a significantly shorter overall survival compared to patients at low risk for disease progression (median OS 13 vs. 38 months, log rank p=0.0001, HR=3.29, 95% CI: 1.77-6.14; FIG. 18B) and these findings were independently validated in cohort 2 (median PFS 3 vs. 8 months, log rank p=0.017, HR=2.73, 95% CI: 1.15-6.45; FIG. 18C).

TABLE 12
Multivariable Cox Proportional Hazards Regression Analysis.

| | Multivariate Cox Proportional | | | |
|---|---|---|---|---|
| | Hazards Model | | | |
| | | Hazard | | p |
| Variable | Coefficient | Ratio | 95% CI | value |
| cTMB | −0.001 | 0.999 | 0.998-1.000 | 0.111 |
| Molecular Smoking | −0.547 | 0.579 | 0.301-1.112 | 0.101 |
| Signature | | | | |
| RTK activating mutation | 0.981 | 2.667 | 1.237-5.750 | 0.012 |
| Unique HLA class I | 0.718 | 2.050 | 0.2765-15.242 | 0.483 |
| alleles-germline | | | | |
| (3-4 vs 5-6) | | | | |

The predictive value of individual biomarkers of response to immunotherapy such as PD-L1 expression and TMB have modest predictive utility across a plethora of studies These analyses showed that the complexities of the predictive value of TMB may be in part attributed to tumor purity and developed a new approach to generate corrected TMB values that more accurately predicted outcome for ICB. These findings are of particular importance for metastatic NSCLC where the majority of tumor samples are obtained by bronchoscopy or core needle biopsies and are therefore subject to tumor purity limitations. While targeted next-generation sequencing may alleviate the tumor purity effect given the higher coverage compared to whole exome sequencing, our findings suggest that TMB values should only be interpreted after taking into consideration the tumor purity of the sample analyzed.

This study found a significant enrichment in activating RTK genomic alterations in non-responding tumors which identified patients with an inferior outcome from immune checkpoint blockade in three independent NSCLC cohorts. This study also found that activating genomic alterations in RTK genes including EGFR, HER2, MET, FGFR1 and IGF1R can be linked to primary resistance to immune checkpoint blockade independent of mutation burden.

Key molecular features identified in this study were combined into a predictive classifier for NSCLC patients treated with ICB. Previous attempts to combine biomarkers have focused on a limited number of features such as TMB and chromosomal imbalance (Roh et al., Science translational medicine 9:3560 (2017)), TMB and immune cell gene expression profiles (Cristescu et al., Science 362:3593 (2018)) or HLA variation and TMB (Chowell et al., Science 359:582-587 (2018); and McGranahan et al., Cell 171:1259-1271 (2017)). The multivariable model described herein incorporates an improved measure of TMB through correction of tumor purity, RTK mutations, molecular smoking signature and HLA genetic variation, highlighting the need for development of integrative platforms that capture the complexities of the cancer-immune system crosstalk.

Methods

Cohort Characteristics

Matched tumor-normal exome sequencing data was obtained from 3,788 patients in TCGA (cancergenome.nih.gov), as outlined in the TCGA publication guidelines cancergenome.nih.gov/publications/publicationguidelines, focusing on tumors that would be relevant for immunotherapy. Cohort 1 consisted of 104 NSCLC patients treated with immune checkpoint blockade at Johns Hopkins Sidney Kimmel Cancer Center and the Nederlands Kanker Instituut. Of these, 15 cases were not included in the final analyses because of tumor purity <10% or absence of matched normal samples. The studies were approved by the Institutional Review Board (IRB) and patients provided written informed consent for sample acquisition for research purposes. Clinical characteristics for all patients are summarized in Table 1. Exome data from a published cohort of NSCLC patients treated with PD1 blockade (cohort 2) were obtained and analyzed to validate key findings from cohort 1 as described elsewhere (see, e.g., Rizvi et al., Science, 348:124-128 (2015); and Wood et al., Science translational medicine 10:7939 (2018)). A publicly available cohort of 240 NSCLC patients treated with ICB was obtained through CBioPortal for Cancer Genomics (MSK, JCO 2018; available online at cbioportal.org/study?id=nsclc_pd1_msk_2018) and used to validate the association of RTK mutations with outcome (cohort 3). A publicly available cohort of 1,661 tumors analyzed by targeted next-generation sequencing was obtained through CBioportal for Cancer Genomics (MSKCC, Nat Genet 51(2):202-206 (2019)) to validate the correlation between TMB and tumor purity in the setting of higher sequencing depth.

Treatment and Assessment of Clinical Response

Eighty patients were treated with anti-PD1 therapy, 7 patients received combination anti-PD1 and anti-CTLA4 therapy and 2 patients were treated with chemotherapy and anti-PD1 therapy. Response was defined as durable clinical benefit if complete, partial response or stable disease was achieved with a duration >6 months. Responding and non-responding tumors, therefore refer to durable clinical benefit and non-durable clinical benefit respectively. Progression-free survival (PFS) and overall survival (OS) were defined as the time elapsed between the date of treatment initiation and the date of disease progression or death from disease, or the date of death, respectively. Ultimately, overall survival was used to determine long-term outcome for cohort 1. Overall survival was not available for cohorts 2 and 3, therefore progression-free survival was used. Response assessments and outcome are shown in detail in Table 1.

TABLE 1
Summary of clinical and tumor sample characteristics.

| | | | | | | | Time- | | | | | PFS | | OS |
|---|---|---|---|---|---|---|---|---|---|---|---|---|---|---|
| | Age | | Stage | | | | point | Path- | | | | censor | | censor |
| | at | | at | | | Ana- | at which | ologic | | | | (0 = | | (0 = |
| | ICB | | ICB | | | tomic | sample | Tumor | | | | censored, | | censored, |
| | initi- | Gen- | initi- | Smoking | | Loca- | was | Purity | | Clinical | | 1 = prog- | | 1 = |
| Patient ID | ation | der | ation | Status | Histology | tion | obtained | (%) | Treatment | Benefit | PFS | ressed) | OS | DOD) |
| CGLU111 | 71 | M | IV | Former | Squamous | liver | prior to | 30% | Anti-PD1 | DCB | 40 | 0 | 40 | 0 |
| | | | | Smoker | Cell | | ICB | | (nivolumab) | | | | | |
| | | | | | Carcinoma | | | | | | | | | |
| CGLU113 | 63 | M | IV | Current | Squamous | R4 | prior to | 40- | Anti-PD1 | NDB | 1 | 1 | 2 | 1 |
| | | | | Smoker | Cell | lymph | | 60% | | | | | | |
| | | | | | Carcinoma | node | ICB | | (nivolumab) | | | | | |
| CGLU115 | 63 | F | IV | Former | Squamous | lung | prior to | 70% | Anti-PD1 | NDB | 2 | 1 | 3 | 0 |
| | | | | Smoker | Cell | | ICB | | (nivolumab) | | | | | |
| | | | | | Carcinoma | | | | | | | | | |
| CGLU116 | 56 | M | IV | Former | Squamous | lung | prior to | NA | Dual ICB (anti- | DCB | 13 | 1 | 17 | 1 |
| | | | | Smoker | Cell | | ICB | | PD1 + | | | | | |
| | | | | | Carcinoma | | | | anti-CTLA4) | | | | | |
| CGLU117 | 56 | M | IV | Current | Adeno- | adrenal | prior to | 80% | Anti-PD1 | DCB | 8 | 1 | 14 | 1 |
| | | | | Smoker | carcinoma | | ICB | | (nivolumab) | | | | | |
| CGLU120 | 58 | F | IV | Former | Adeno- | lung | prior to | 60- | Anti-PD1 | NDB | 6 | 1 | 17 | 1 |
| | | | | Smoker | carcinoma | | ICB | 70% | (nivolumab) | | | | | |
| CGLU121 | 47 | F | IV | Never | Adeno- | lung | prior to | NA | Anti-PD1 | NDB | 1 | 1 | 9 | 1 |
| | | | | Smoker | carcinoma | | ICB | | (nivolumab) | | | | | |
| CGLU124 | 50 | F | IV | Never | Adeno- | lymph | prior to | 20- | Anti-PD1 | NDB | 2 | 1 | 6 | 1 |
| | | | | Smoker | carcinoma | node | ICB | 30% | (nivolumab) | | | | | |
| CGLU125 | 76 | F | IV | Former | Adeno- | lung | prior to | 20- | Anti-PD1 | DCB | 23 | 1 | 51 | 0 |
| | | | | Smoker | carcinoma | | ICB | 30% | (nivolumab) | | | | | |
| CGLU126 | 73 | F | IV | Never | LCNEC | lung | prior to | 90% | Anti-PD1 | NDB | 5 | 1 | 15 | 1 |
| | | | | Smoker | | | ICB | | (nivolumab) | | | | | |
| CGLU127 | 59 | F | IV | Former | Adeno- | lung | prior to | 70% | Anti-PD1 | DCB | 10 | 1 | 25 | 1 |
| | | | | Smoker | carcinoma | | ICB | | (nivolumab) | | | | | |
| CGLU128 | 65 | F | IV | Never | Adeno- | adrenal | prior to | 20- | Anti-PD1 | NDB | 5 | 1 | 38 | 1 |
| | | | | Smoker | carcinoma | | ICB | 30% | (nivolumab) | | | | | |
| CGLU129 | 62 | M | IV | Former | Adeno- | soft | prior to | 30- | Anti-PD1 | NDB | 2 | 1 | 16 | 1 |
| | | | | Smoker | carcinoma | tissue | ICB | 40% | (nivolumab) | | | | | |
| CGLU130 | 74 | F | IV | Former | Adeno- | N/A | prior to | 40% | Anti-PD1 | NDB | 4 | 1 | 5 | 0 |
| | | | | Smoker | carcinoma | | ICB | | (nivolumab) | | | | | |
| CGLU131 | 57 | M | IV | Never | Adeno- | lung | prior to | 50% | Anti-PD1 | DCB | 7 | 1 | 13 | 1 |
| | | | | Smoker | carcinoma | | ICB | | (nivolumab) | | | | | |
| CGLU132 | 63 | M | IV | Never | Adeno- | lung | prior to | 20% | Anti-PD1 | NDB | 2 | 1 | 6 | 1 |
| | | | | Smoker | carcinoma | | ICB | | (nivolumab) | | | | | |
| CGLU133 | 61 | M | IV | Former | Squamous | pleural | prior to | 80% | Anti-PD1 | DCB | 93 | 0 | 93 | 0 |
| | | | | Smoker | Cell | nodule | ICB | | (nivolumab) | | | | | |
| | | | | | Carcinoma | | | | | | | | | |
| CGLU134 | 72 | F | IV | Former | Adeno- | lung | prior to | NA | Anti-PD1 | DCB | 57 | 0 | 57 | 0 |
| | | | | Smoker | carcinoma | | ICB | | (nivolumab) | | | | | |
| CGLU135 | 59 | M | IV | Former | Squamous | lung | prior to | NA | Anti-PD1 | DCB | 23 | 1 | 46 | 0 |
| | | | | Smoker | Cell | | ICB | | (nivolumab) | | | | | |
| | | | | | Carcinoma | | | | | | | | | |
| CGLU159 | 63 | F | IV | Former | Squamous | pleura | prior to | 20% | Anti-PD1 | NDB | 3 | 1 | 5 | 1 |
| | | | | Smoker | Cell | | ICB | | (nivolumab) | | | | | |
| | | | | | Carcinoma | | | | | | | | | |
| CGLU160 | 87 | M | IV | Former | Adeno- | lung | prior to | 50% | Anti-PD1 | DCB | 13 | 0 | 13 | 1 |
| | | | | Smoker | carcinoma | | ICB | | (nivolumab) | | | | | |
| CGLU162 | 78 | F | IV | Former | Squamous | lung | prior to | 40% | Anti-PD1 | DCB | 7 | 1 | 7 | 0 |
| | | | | Smoker | Cell | | ICB | | (nivolumab) | | | | | |
| | | | | | Carcinoma | | | | | | | | | |
| CGLU163 | 72 | M | IV | Former | Squamous | lung | prior to | 50% | Anti-PD1 | NDB | 3 | 1 | 39 | 0 |
| | | | | Smoker | Cell | | ICB | | (nivolumab) | | | | | |
| | | | | | Carcinoma | | | | | | | | | |
| CGLU168 | 88 | M | IV | Former | Adeno- | lung | prior to | 80% | Anti-PD1 | DCB | 7 | 1 | 13 | 0 |
| | | | | Smoker | carcinoma | | ICB | | (nivolumab) | | | | | |
| CGLU169 | 62 | F | IV | Former | Adeno- | lymph | prior to | 20% | Anti-PD1 | NDB | 1 | 0 | 1 | 1 |
| | | | | Smoker | carcinoma | node | ICB | | (nivolumab) | | | | | |
| CGLU172 | 58 | F | IV | Former | Adeno- | lung | prior to | 90% | Anti-PD1 | NDB | 4 | 1 | 9 | 1 |
| | | | | Smoker | carcinoma | | ICB | | (nivolumab) | | | | | |
| CGLU178 | 68 | M | IV | Never | Other | lung | prior to | 20% | Anti-PD1 | N/A | 1 | 0 | 1 | 0 |
| | | | | Smoker | | | ICB | | (nivolumab) | | | | | |
| CGLU180 | 76 | M | IV | Former | Other | lung | prior to | 20% | Anti-PD1 | DCB | 11 | 0 | 11 | 0 |
| | | | | Smoker | | | ICB | | (nivolumab) | | | | | |
| CGLU181 | 55 | M | IV | Former | Squamous | lung | prior to | 30- | Anti-PD1 | DCB | 7 | 1 | 16 | 0 |
| | | | | Smoker | Cell | | ICB | 50% | (nivolumab) | | | | | |
| | | | | | Carcinoma | | | | | | | | | |
| CGLU185 | 56 | F | IV | Former | Adeno- | lymph | prior to | NA | Anti-PD1 | DCB | 12 | 1 | 38 | 0 |
| | | | | Smoker | carcinoma | node | ICB | | (nivolumab) | | | | | |
| CGLU187 | 59 | F | IV | Former | Adeno- | N/A | prior to | 30% | Anti-PD1 | NDB | 2 | 1 | 2 | 1 |
| | | | | Smoker | carcinoma | | ICB | | (nivolumab) | | | | | |
| CGLU189 | 46 | F | IV | Never | Adeno- | lymph | prior to | 80% | Anti-PD1 | NDB | 2 | 1 | 26 | 1 |
| | | | | Smoker | carcinoma | node | ICB | | (nivolumab) | | | | | |
| CGLU193 | 65 | M | IV | Former | LCNEC | lung | prior to | 50% | Anti-PD1 | NDB | 1 | 1 | 2 | 1 |
| | | | | Smoker | | | ICB | | (nivolumab) | | | | | |
| CGLU197 | 68 | M | IV | Never | Squamous | lung | prior to | 30% | Anti-PD1 | DCB | 8 | 1 | 38 | 0 |
| | | | | Smoker | Cell | | ICB | | (nivolumab) | | | | | |
| | | | | | Carcinoma | | | | | | | | | |
| CGLU198 | 60 | F | IV | Never | Adeno- | lung | prior to | NA | Anti-PD1 | NDB | 3 | 0 | 3 | 1 |
| | | | | Smoker | carcinoma | | ICB | | (nivolumab) | | | | | |
| CGLU199 | 61 | M | IV | Never | Squamous | chest | prior to | 50% | Anti-PD1 | NDB | 2 | 1 | 9 | 1 |
| | | | | Smoker | Cell | wall | ICB | | (nivolumab) | | | | | |
| | | | | | Carcinoma | | | | | | | | | |
| CGLU200 | 54 | M | IV | Never | Adeno- | lymph | prior to | NA | Anti-PD1 | NDB | 1 | 0 | 1 | 1 |
| | | | | Smoker | carcinoma | node | ICB | | (nivolumab) | | | | | |
| CGLU201 | 51 | M | IV | Current | Adeno- | lung | prior to | 70% | Anti-PD1 | NDB | 2 | 1 | 3 | 0 |
| | | | | Smoker | carcinoma | | ICB | | (nivolumab) | | | | | |
| CGLU203 | 65 | F | IV | Former | Adeno- | iliac | prior to | 40% | Anti-PD1 | NDB | 2 | 1 | 4 | 0 |
| | | | | Smoker | carcinoma | wing | ICB | | (nivolumab) | | | | | |
| CGLU208 | 67 | F | IV | Former | Adeno- | lymph | prior to | 60% | Anti-PD1 | DCB | 20 | 1 | 25 | 1 |
| | | | | Smoker | carcinoma | node | ICB | | (nivolumab) | | | | | |
| CGLU211 | 71 | F | IV | Current | Squamous | lung | prior to | 40% | Anti-PD1 | DCB | 11 | 1 | 28 | 0 |
| | | | | Smoker | Cell | | ICB | | (nivolumab) | | | | | |
| | | | | | Carcinoma | | | | | | | | | |
| CGLU212 | 57 | M | IV | Former | Adeno- | lung | prior to | 40% | Anti-PD1 | DCB | 12 | 0 | 12 | 0 |
| | | | | Smoker | carcinoma | | ICB | | (nivolumab) | | | | | |
| CGLU213 | 84 | F | IV | Former | Adeno- | lung | prior to | 60% | Anti-PD1 | NDB | 1 | 1 | 8 | 0 |
| | | | | Smoker | carcinoma | | ICB | | (nivolumab) | | | | | |
| CGLU227 | 68 | M | IV | Former | Adeno- | N/A | prior to | NA | Anti-PD1 | DCB | 10 | 1 | 22 | 0 |
| | | | | Smoker | carcinoma | | ICB | | (nivolumab) | | | | | |
| CGLU229 | 69 | M | IV | Never | Adeno- | N/A | prior to | 80% | Anti-PD1 | NDB | 2 | 1 | 3 | 1 |
| | | | | Smoker | carcinoma | | ICB | | (nivolumab) | | | | | |
| CGLU230 | 62 | F | IV | Never | Adeno- | N/A | prior to | 80% | Anti-PD1 | NDB | 6 | 1 | 13 | 0 |
| | | | | Smoker | carcinoma | | ICB | | (nivolumab) | | | | | |
| CGLU231 | 50 | M | IV | Former | Squamous | N/A | prior to | 20% | Anti-PD1 | NDB | 4 | 1 | 18 | 1 |
| | | | | Smoker | Cell | | ICB | | (nivolumab) | | | | | |
| | | | | | Carcinoma | | | | | | | | | |
| CGLU232 | 73 | M | IV | Never | Adeno- | N/A | prior to | 30% | Anti-PD1 | NDB | 2 | 1 | 3 | 1 |
| | | | | Smoker | carcinoma | | ICB | | (nivolumab) | | | | | |
| CGLU233 | 80 | M | IV | Former | Squamous | N/A | prior to | 35% | Anti-PD1 | DCB | 14 | 1 | 17 | 0 |
| | | | | Smoker | Cell | | ICB | | (nivolumab) | | | | | |
| | | | | | Carcinoma | | | | | | | | | |
| CGLU240 | 60 | M | IV | Former | Squamous | N/A | prior to | 80% | Anti-PD1 | DCB | 13 | 1 | 13 | 0 |
| | | | | Smoker | Cell | | ICB | | (nivolumab) | | | | | |
| | | | | | Carcinoma | | | | | | | | | |
| CGLU243 | 73 | F | IV | Never | Adeno- | N/A | prior to | 10% | Anti-PD1 | NDB | 2 | 1 | 5 | 1 |
| | | | | Smoker | carcinoma | | ICB | | (nivolumab) | | | | | |
| CGLU244 | 63 | M | IV | Former | Squamous | brain | prior to | 70% | Anti-PD1 | NDB | 5 | 1 | 8 | 1 |
| | | | | Smoker | Cell | | ICB | | (pembrolizumab) | | | | | |
| | | | | | Carcinoma | | | | | | | | | |
| CGLU246 | 77 | M | IV | Former | Squamous | bone | prior to | 60% | Dual ICB (anti- | DCB | 20 | 0 | 20 | 0 |
| | | | | Smoker | Cell | | ICB | | PD1 + | | | | | |
| | | | | | Carcinoma | | | | anti-CTLA4) | | | | | |
| CGLU247 | 82 | M | IV | Former | Adeno- | lung | prior to | 80% | Anti-PD1 | NDB | 3 | 0 | 3 | 1 |
| | | | | Smoker | carcinoma | | ICB | | (nivolumab) | | | | | |
| CGLU248 | 67 | M | IV | Former | Squamous | lung | prior to | 50% | Dual ICB (anti- | NDB | 2 | 1 | 6 | 1 |
| | | | | Smoker | Cell | | ICB | | PD1 + | | | | | |
| | | | | | Carcinoma | | | | anti-CTLA4) | | | | | |
| CGLU252 | 67 | F | IV | Former | Adeno- | lung | prior to | 40% | Dual ICB (anti- | DCB | 10 | 0 | 10 | 1 |
| | | | | Smoker | carcinoma | | ICB | | PD1 + | | | | | |
| | | | | | | | | | anti-CTLA4) | | | | | |
| CGLU257 | 74 | M | IV | Never | Adeno- | lung | prior to | 40% | Dual ICB (anti- | NDB | 1 | 1 | 15 | 1 |
| | | | | Smoker | carcinoma | | ICB | | PD1 + | | | | | |
| | | | | | | | | | anti-CTLA4) | | | | | |
| CGLU260 | 79 | M | IV | Former | Adeno- | liver | prior to | 70% | Anti-PD1 | NDB | 1 | 1 | 1 | 0 |
| | | | | Smoker | carcinoma | | ICB | | (nivolumab) | | | | | |
| CGLU262 | 72 | M | IV | Former | Adeno- | brain | prior to | 90% | Anti-PD1 | NDB | 2 | 1 | 19 | 0 |
| | | | | Smoker | carcinoma | | ICB | | (nivolumab) | | | | | |
| CGLU266 | 71 | F | IV | Never | Adeno- | lung | prior to | 70% | Anti-PD1 | DCB | 23 | 1 | 43 | 0 |
| | | | | Smoker | carcinoma | | ICB | | (nivolumab) | | | | | |
| CGLU268 | 51 | F | IV | Former | LCNEC | lung | prior to | 60% | Dual ICB (anti- | DCB | 21 | 0 | 21 | 0 |
| | | | | Smoker | | | ICB | | PD1 + | | | | | |
| | | | | | | | | | anti-CTLA4) | | | | | |
| CGLU270 | 61 | M | IV | Former | Adeno- | brain | prior to | 90% | Anti-PD1 | DCB | 29 | 0 | 29 | 0 |
| | | | | Smoker | carcinoma | | ICB | | (nivolumab) | | | | | |
| CGLU274 | 74 | F | IV | Current | Squamous | lymph | prior to | 60% | Anti-PD1 | DCB | 7 | 0 | 7 | 0 |
| | | | | Smoker | Cell | node | ICB | | (pembrolizumab) | | | | | |
| | | | | | Carcinoma | | | | | | | | | |
| CGLU286 | 48 | M | IV | Never | Adeno- | media- | prior to | 50- | Dual ICB (anti- | NDB | 6 | 1 | 14 | 1 |
| | | | | Smoker | carcinoma | stinal | ICB | 60% | PD1 + | | | | | |
| | | | | | | mass | | | anti-CTLA4) | | | | | |
| CGLU287 | 73 | F | IV | Former | Adeno- | lung | prior to | 60- | Anti-PD1 | NDB | 3 | 1 | 3 | 1 |
| | | | | Smoker | carcinoma | | ICB | 70% | (nivolumab) | | | | | |
| CGLU288 | 54 | F | IV | Never | Adeno- | brain | prior to | 50% | Anti-PD1 | NDB | 1 | 1 | 5 | 1 |
| | | | | Smoker | carcinoma | | ICB | | (pembrolizumab) | | | | | |
| CGLU289 | 64 | F | IV | Current | Adeno- | brain | prior to | 90% | Anti-PD1 | DCB | 15 | 0 | 15 | 1 |
| | | | | Smoker | carcinoma | | ICB | | (pembrolizumab) | | | | | |
| CGLU295 | 77 | M | IV | Former | Squamous | lymph | prior to | 50% | Anti-PD1 | NDB | 4 | 0 | 4 | 1 |
| | | | | Smoker | Cell | node | ICB | | (nivolumab) | | | | | |
| | | | | | Carcinoma | | | | | | | | | |
| CGLU299 | 58 | F | IV | Former | Squamous | lymph | prior to | 50% | Anti-PD1 | NDB | 3 | 1 | 7 | 0 |
| | | | | Smoker | Cell | node | ICB | | (nivolumab) | | | | | |
| | | | | | Carcinoma | | | | | | | | | |
| CGLU304 | 81 | M | IV | Former | Adeno- | pleural | prior to | 45% | Anti-PD1 | NDB | 2 | 1 | 8 | 1 |
| | | | | Smoker | carcinoma | fluid | ICB | | (nivolumab) | | | | | |
| CGLU305 | 55 | F | IV | Former | Adeno- | lung | prior to | 7% | Anti-PD1 | NDB | 4 | 1 | 16 | 0 |
| | | | | Smoker | carcinoma | | ICB | | (pembrolizumab) | | | | | |
| CGLU307 | 66 | F | IV | Former | Adeno- | bone | resistant | 20% | Anti-PD1 | NDB | 2 | 1 | 19 | 0 |
| | | | | Smoker | carcinoma | | tumor | | (nivolumab) | | | | | |
| CGLU309 | 85 | M | IV | Former | Adeno- | lymph | prior to | 70% | Anti-PD1 | NDB | 1 | 1 | 3 | 1 |
| | | | | Smoker | carcinoma | node | ICB | | (pembrolizumab) | | | | | |
| CGLU310 | 56 | F | IV | Current | Adeno- | lymph | prior to | 80% | Anti-PD1 | DCB | 14 | 0 | 14 | 0 |
| | | | | Smoker | carcinoma | node | ICB | | (pembrolizumab) | | | | | |
| CGLU311 | 69 | F | IV | Current | Adeno- | lymph | prior to | 65% | Anti-PD1 | DCB | 16 | 0 | 16 | 0 |
| | | | | Smoker | carcinoma | node | ICB | | (pembrolizumab) | | | | | |
| CGLU327 | 75 | M | IV | Former | Adeno- | lung | prior to | 35% | Anti-PD1 | DCB | 14 | 0 | 14 | 0 |
| | | | | Smoker | carcinoma | | ICB | | (pembrolizumab) | | | | | |
| CGLU329 | 68 | F | IV | Former | Adeno- | lung | prior to | 20% | Anti-PD1 | DCB | 14 | 0 | 14 | 0 |
| | | | | Smoker | carcinoma | | ICB | | (pembrolizumab) | | | | | |
| CGLU334 | 72 | M | IV | Former | Adeno- | lung | prior to | 40% | Anti-PD1 | DCB | 29 | 0 | 29 | 0 |
| | | | | Smoker | carcinoma | | ICB | | (nivolumab) | | | | | |
| CGLU337 | 58 | M | IV | Former | Adeno- | bone | prior to | 25% | Anti-PD1 + | DCB | 14 | 0 | 14 | 0 |
| | | | | Smoker | carcinoma | | ICB | | Chemotherapy | | | | | |
| CGLU341 | 64 | F | IV | Current | Adeno- | pleura | prior to | 65% | Anti-PD1 + | DCB | 13 | 0 | 13 | 0 |
| | | | | Smoker | carcinoma | | ICB | | Chemotherapy | | | | | |
| CGLU348 | 63 | F | IV | Former | Squamous | lung | prior to | 45% | Anti-PD1 | N/A | 3 | 0 | 3 | 0 |
| | | | | Smoker | Cell | | ICB | | (pembrolizumab) | | | | | |
| | | | | | Carcinoma | | | | | | | | | |
| CGLU389 | 61 | M | IV | Former | Adeno- | lung | prior to | 45% | Anti-PD1 | NDB | 3 | 1 | 9 | 0 |
| | | | | Smoker | carcinoma | | ICB | | (pembrolizumab) | | | | | |
| CGLU436 | 52 | M | IV | Never | Adeno- | bone | prior to | 50% | Anti-PD1 | NDB | 3 | 1 | 3 | 1 |
| | | | | Smoker | carcinoma | | ICB | | (pembrolizumab) | | | | | |
| CGLU510 | 61 | F | IV | Former | Adeno- | liver | resistant | 70% | Anti-PD1 | DCB | 11 | 1 | 16 | 0 |
| | | | | Smoker | carcinoma | | tumor | | (nivolumab) | | | | | |
| CGLU512 | 66 | F | IV | Former | Adeno- | lung | prior to | 70% | Anti-PD1 | DCB | 9 | 1 | 27 | 0 |
| | | | | Smoker | carcinoma | | ICB | | (nivolumab) | | | | | |
| CGLU514 | 73 | F | IV | Former | Adeno- | adnexa | resistant | 90% | Anti-PD1 | DCB | 10 | 1 | 28 | 0 |
| | | | | Smoker | carcinoma | | tumor | | (nivolumab) | | | | | |
| CGLU515 | 74 | M | IV | Former | Adeno- | soft | prior to | 80% | Anti-PD1 | DCB | 19 | 1 | 31 | 0 |
| | | | | Smoker | carcinoma | tissue | ICB | | (nivolumab) | | | | | |
| CGLU519 | 54 | M | IV | Former | Adeno- | lung | prior to | 50% | Anti-PD1 | NDB | 2 | 1 | 3 | 1 |
| | | | | Smoker | carcinoma | | ICB | | (nivolumab) | | | | | |
| CGLU521 | 46 | F | IV | Former | Adeno- | adrenal | prior to | 80% | Anti-PD1 | DCB | 10 | 0 | 10 | 0 |
| | | | | Smoker | carcinoma | | ICB | | (nivolumab) | | | | | |

ICB; immune checkpoint blockade,
M; male,
F; female,
LCNEC; large cell neoendocrine carcinoma,
DCB; durable clinical benefit,
NDB; non durable clinical benefit,
PFS; progression-free survival,
OS; overall survival

Sample Preparation and Whole Exome Sequencing

Whole exome sequencing was performed on pre-immunotherapy tumor and matched normal samples, with the exception of 3 cases for which tumor from the time of resistance to therapy was analyzed (Table 1). Tumor samples underwent pathological review for confirmation of lung cancer diagnosis and assessment of tumor cellularity; histology, anatomic location of the lesion analyzed and pathologic tumor purity are shown in Table 1. Slides from each FFPE block were macrodissected to remove contaminating normal tissue. Matched normal samples were provided as peripheral blood. DNA was extracted from patients' tumors and matched peripheral blood using the Qiagen DNA FFPE and Qiagen DNA blood mini kit respectively (Qiagen, CA). Fragmented genomic DNA from tumor and normal samples used for Illumina TruSeq library construction (Illumina, San Diego, Calif.) and exonic regions were captured in solution using the Agilent SureSelect v.4 kit (Agilent, Santa Clara, Calif.) according to the manufacturers' instructions as described elsewhere (see, e.g., Anagnostou et al., Cancer discovery 7:264-276 (2017)). Paired-end sequencing, resulting in 100 bases from each end of the fragments for the exome libraries was performed using Illumina HiSeq 2000/2500 instrumentation (Illumina, San Diego, Calif.). The mean depth of total and distinct coverage for the pre-treatment tumors were 231× and 144×, allowing identification of sequence alterations and copy number changes in >20,000 genes (Tables 2, 3 and 6).

Primary Processing of Exome Data and Identification of Putative Somatic Mutations

Somatic mutations were identified using VariantDx custom software for identifying mutations in matched tumor and normal samples as described elsewhere (see, e.g., Jones et al., Science translational medicine 7, 283ra253 (2015)). Prior to mutation calling, primary processing of sequence data for both tumor and normal samples were performed using Illumina CASAVA software (version 1.8), including masking of adapter sequences. Sequence reads were aligned against the human reference genome (version hg19) using ELAND with additional realignment of select regions using the Needleman-Wunsch method as described elsewhere (see, e.g., Needleman et al., J Mol Biol 48:443-453 (1970)). Candidate somatic mutations, consisting of point mutations, insertions, and deletions were then identified using VariantDx across the whole exome. VariantDx examines sequence alignments of tumor samples against a matched normal while applying filters to exclude alignment and sequencing artifacts. In brief, an alignment filter was applied to exclude quality failed reads, unpaired reads, and poorly mapped reads in the tumor. A base quality filter was applied to limit inclusion of bases to those with reported Phred quality score >30 for the tumor and >20 for the normal. A mutation in the pre or post treatment tumor samples was identified as a candidate somatic mutation only when (1) distinct paired reads contained the mutation in the tumor; (2) the fraction of distinct paired reads containing a particular mutation in the tumor was at least 10% of the total distinct read pairs and (3) the mismatched base was not present in >1% of the reads in the matched normal sample as well as not present in a custom database of common germline variants derived from dbSNP and (4) the position was covered in both the tumor and normal. Mutations arising from misplaced genome alignments, including paralogous sequences, were identified and excluded by searching the reference genome. Candidate somatic mutations were further filtered based on gene annotation to identify those occurring in protein coding regions. Functional consequences were predicted using snpEff and a custom database of CCDS, RefSeq and Ensembl annotations using the latest transcript versions available on hg19 from UCSC (genome.ucsc.edu/). Predictions were ordered to prefer transcripts with canonical start and stop codons and CCDS or Refseq transcripts over Ensembl when available. Finally, mutations were filtered to exclude intronic and silent changes, while retaining mutations resulting in missense mutations, nonsense mutations, in-frame and frameshift insertions and deletions, or splice site alterations. Somatic mutations were annotated against the set of mutations in COSMIC (v84) database, and the number of samples with identical amino acid change were reported. Mutations were characterized as hotspots when the same amino acid change was reported in at least 10 tumor samples in COSMIC v84 database. Missense mutations were evaluated for their potential as cancer drivers by CHASMplus (Tokheim et al., bioRxiv dx.doi.org/10.1101/010876 (2018)). For the differential enrichment analysis between patients with durable and non-durable clinical benefit, only genomic alterations with known cancer initiating/promoting functional consequences independent of observed frequency and hotspots for oncogenes and truncating/loss-of-function mutations for tumor suppressor genes were considered.

For the TCGA cohort, WES-derived somatic mutation calls from the TCGA PanCancer Atlas MC3 project were retrieved from the NCI Genomic Data Commons (gdc.cancer.gov/about-data/publications/mc3-2017). The MC3 mutation call set is the result of application of a uniform analysis pipeline including a standardized set of six mutation callers and an array of automated filters to all the entire TCGA exome data. Mutation calls in cohort 2 were obtained from re-analysis of the original calls and consequence prediction was performed using CRAVAT (Masica et al., Cancer Res 77, e35-e38 (2017)). TMB scores for the cohort of 1,661 tumors were retrieved from the original publication and refer to the total number of somatic mutations identified normalized to the exonic coverage of the targeted panel used in megabases (Samstein et al., Nature genetics, 51(2):202-206 (2019)).

Neoantigen Prediction and Feature Characterization

To assess the immunogenicity of somatic mutations, exome data combined with each individual patient's MHC class I haplotype were applied in a neoantigen prediction platform that evaluates binding of somatic peptides to class I WIC, antigen processing, self-similarity and gene expression. Detected somatic mutations, consisting of nonsynonymous single base substitutions, insertions and deletions, were evaluated for putative neoantigens using the ImmunoSelect-R pipeline (Personal Genome Diagnostics, Baltimore, Md.) as described elsewhere (see, e.g., Anagnostou et al., Cancer discovery 7:264-276 (2017)). For single base substitutions, ImmunoSelect-R performs a comprehensive assessment of paired somatic and wild type peptides 8-11 amino acids in length at every position surrounding a somatic mutation. In the case of frameshifts, all peptides 8-11 amino acids encompassing the new protein sequence resulting from the frameshift alteration were considered.

To accurately infer a patient's germline HLA 4-digit allele genotype, whole-exome-sequencing data from paired tumor/normal samples were first aligned to a reference allele set, which was then formulated as an integer linear programming optimization procedure to generate a final genotype by OptiType v1.0.44. The HLA genotype served as input to netMHCpan to predict the WIC class I binding potential of each somatic and wild-type peptide (IC50 nM), with each peptide classified as a strong binder (SB), weak binder (WB) or non-binder (NB) as described elsewhere (see, e.g., Nielsen et al., Genome Med 8:33 (2016); Lundegaard et al., Nucleic Acids Res 36:W509-512 (2008); and Lundegaard et al., Bioinformatics 24:1397-1398 (2008)). Peptides were further evaluated for antigen processing (netCTLpan48) and were classified as cytotoxic T lymphocyte epitopes (E) or non-epitopes (NA). Paired somatic and wild-type peptides were assessed for self-similarity based on MHC class I binding affinity. Neoantigen candidates meeting an IC50 affinity <5000 nM were subsequently ranked based on MHC binding and T-cell epitope classifications. A single MANA per mutation was selected based on their MHC affinity and neoantigen candidates with an MHC affinity <500 nM were further selected to estimate the neoantigen tumor burden and used for downstream analyses. Tumor-associated expression levels derived from TCGA were used to generate a final ranking of candidate immunogenic peptides. MANAs were further characterized based on their immunogenic potential by selecting neopeptides with high MHC affinity for which their wild type counterpart predicted not to bind MHC class I molecules (fit MANA: MHC affinity for mutant peptide <50 nM and for wild type peptide >1000 nM). For MANAs stemming from frameshift mutations, the length of the resulting protein until a stop codon was reached was considered, as a longer novel amino acid sequence would have the potential to generate more immunogenic neoantigens. Sequences more prone to undergo nonsense mediated decay were subsequently filtered out as described elsewhere (see, e.g., Balasubramanian et al., Nature communications 8:382 (2017)), during this process aberrant transcripts are typically removed at the mRNA level and therefore would not stand a chance of occurring despite the presence of bioinformatic predictions. The percentage of frameshift mutations undergoing nonsense mediated decay is shown in FIG. 12. Frameshift MANAs were interrogated for homology to microbial and viral antigens by matching the peptide sequence to peptides in the Immune Epitope Database (IEDB, www.iedb.org), requiring a match of >80% for identity and >75% for length.

Mutational Signatures

Mutational signatures were extracted based on the fraction of coding point mutations in each of 96 trinucleotide contexts and estimated the contribution of each signature to each tumor sample using the deconstructSigs R package as described elsewhere (see, e.g., Viray et al., Archives of pathology & laboratory medicine 137:1545-1549 (2013); and Anagnostou et al., Cancer discovery 7:264-276 (2017)). To evaluate the impact of the total number of observed single base substitutions on detection of a smoking signature within a tumor sample, in-silico dilution experiments were performed utilizing somatic mutation data from 985 NSCLC samples from the TCGA PanCancer Atlas MC3 project. A total of 76 tumors (64 LUAD and 12 LUSC, with average patient pack years of 43.8 and 32.8, respectively) with mutational loads >250 (requiring a minimum 10% MAF and at least 4 variant supporting reads per mutation) and a detected smoking signature with >75% contribution were diluted in silico by subsampling to lower mutation counts from 5 up to 100. For each round of subsampling, tumor mutations were re-evaluated for a smoking signature using the deconstructSigs package. Reductions in the smoking signature and overall percentage deviation from the original smoking signature percent contribution were then assessed in the sample.

Copy Number Analyses, Tumor Purity and Ploidy Assessment

The somatic copy number profile and the extent of aneuploidy in each tumor were estimated using whole exome sequencing data as follows. First, the relative copy number profile of each tumor sample was determined by evaluating the number of reads mapping to exonic and intronic regions (bins) of the genome while correcting them for confounding factors such as region size, GC content, and sequence complexity. The corrected density profile in each tumor sample was then compared to a reference generated by processing a panel of normal samples in a similar manner to define log copy ratio values which reflect the relative copy number profile of each genomic region. Next, circular binary segmentation (CBS) was applied to bin-level copy ratio values to reduce the inherent noise associated with stochastic read count variation and to enable accurate assessment of copy number breakpoints; i.e. boundaries between genomic segments with distinct somatic copy number. Finally, a genome-wide analysis of segmental copy ratio values combined with minor allele frequency of heterozygous SNPs overlapping the segments, implemented as an in-house pipeline, yielded an estimate of tumor purity and ploidy. In brief, the model exhaustively evaluated all plausible combinations of tumor purity and ploidy and returned the optimal combination of the two parameters using a maximum likelihood approach. The performance of this platform was compared against FACETS on a collection of 97 NSCLC tumors and the two methods provided similar estimates of tumor purity (r=0.94, p-value <2.2e-16) and ploidy (r=0.66, p-value=1.489e-13). The estimated purity and ploidy of the tumor sample were subsequently used to determine the allele specific copy number of genome segment by selecting the combination of total and minor copy number that best approximate the segment's log copy ratio and average minor allele frequency as described elsewhere (see, e.g., Anagnostou et al., Cancer discovery 7:264-276 (2017)).

Focal amplifications and homozygous deletions were determined as segments of the genome with length ≤3 Mbp and total copy number greater than or equal to three times ploidy of the genome (amplification), or total copy number of zero (deletion). To increase the specificity of this approach, a set of blacklisted regions was created from a panel of 96 healthy control samples. For each healthy sample, a weighted mean and weighted standard deviation was calculated from segment means obtained from the circular binary segmentation algorithm on copy ratio values, weighted by the number of bins supporting each segment. Genomic intervals in each healthy sample with a segment mean greater than 3 standard deviations away from the mean were added to the blacklist. Focal alterations where >50% of the segment overlapped a blacklisted region in at least 2 healthy control samples were dropped. In addition, segments supported by less than 5 bins and also segments from GC-rich and GC-poor regions of the genome where more than 50% of bins supporting a segment had a GC-content of less than 35% or greater than 70% were excluded.

Several measures of tumor aneuploidy were calculated including the fraction of the genome with loss of heterozygosity (LOH: complete loss of the minor allele), and allelic imbalance (AI: inequality of major and minor allele copy number). In each tumor sample, the modal copy number was determined as the most prevalent total copy number value across the genome. The fraction of the genome with total copy number-CN different from this modal value was calculated and referred to as Non-modal CN Fraction. This measure of aneuploidy is equal to zero for a euploid genome, and increases as the tumor genome accumulates copy number aberrations. Finally, the fraction of the genome at each observed total copy number value was determined, and applied the concept of entropy from information theory to quantify the amount of uncertainty in the assignment of total copy number for each genomic segment. Genome CN Entropy is at its minimum when the entire genome is at a single total copy number, and reaches its maximum when all the observed total copy number levels represent equal fractions of the genome; e.g. 25% of the genome at n=1, 2, 3, and 4.

For a subset of cases (n=14 in cohort 1 and n=10 in cohort 2) where the pipeline could not determine the purity and ploidy due to low tumor purity, technical noise, or copy-number heterogeneity, a mutation-based measure of tumor purity based on the median of mutant allele fractions was used to derive an approximate measure of tumor purity. Tumor purity estimates from copy number analysis above were combined with these mutation-based estimates to define the “Adjusted Tumor Purity” measure.

Evaluation of Tumor Purity in TCGA Samples

Consensus tumor purity estimates from four independent methods were obtained for TCGA samples as described elsewhere (see, e.g., Aran et al., Nature communications 6:8971 (2015)). The analysis were restricted to 3,788 TCGA samples from 7 tumor types (BLCA, BRCA, COAD, HNSC, KIRC, LUAD, LUSC, and SKCM) that had both MC3 mutation calls and a consensus tumor purity estimate. For each cancer type, we computed the Pearson correlation between the total number of mutations called in each sample and tumor purity (FIG. 2). Tumor purity for the cohort of 1,661 tumors were retrieved from the original publication (Samstein et al., Nature genetics, 51(2):202-206 (2019)).

Mutation Clonality Assessment

Mutant allele frequency, ploidy and purity were incorporated to estimate mutation cellular fraction that is the fraction of cancer cells that harbor a specific mutation. SCHISM56 was applied to determine the mutation cellular fraction based on the observed variant allele frequency, estimated copy number, and sample purity by following an approach similar to that described elsewhere (see, e.g., Anagnostou et al., Cancer discovery 7:264-276 (2017)). Briefly, the expected mutant allele frequency (Vₑₓₚ) of a mutation with mutation cellular fraction (CF) present in m copies (mutation multiplicity), at a locus with total copy number (n_{T}) in the tumor sample and total copy number (n N) in the matched normal sample, with purity (α) can be calculated as

$${V}_{exp} = \frac{m C F α}{α {n}_{T} + ( 1 - α ) {n}_{N}}$$

Where m indicates multiplicity, i.e. the number of mutant copies present in the cancer cells. A confidence interval for variable Vₑₓₚ can be derived based on the observed distinct mutant counts and distinct coverage assuming a binomial process. Substitution of this value in the above equation resulted in a confidence interval for the product of the two unknown variables m and CF. Finally, the following set of rules were applied to determine the mutation cellular fraction: (1) For clonal mutations (CF=1), the product m*CF only assumes integer values; therefore, if the confidence interval includes an integer value, that value is equal to the multiplicity of the mutation and the mutation is clonal (CF=1). (2) For mutations where the upper bound of the confidence interval form*CF is below 1, multiplicity is assumed to be 1. If the point estimate for CF is within a tolerance threshold (0.25) of 1.0, the mutation is assumed to be clonal and CF is substituted by 1.0. Otherwise, the mutation is deemed subclonal. (3) For mutations where the confidence interval for m*CF does not encompass an integer number and the entire interval exceeds 1.0, it is plausible to assume a multiplicity greater than 1.0. In this case, the multiplicity is set to smallest integer value such that the confidence value for CF falls within the expected interval of [0, 1]. This procedure results in a point estimate for CF. Similar to (2), if the point estimate is within a tolerance threshold (0.25) of 1.0, the mutation is assumed to be clonal and CF is substituted by 1.0; otherwise, the mutation is considered subclonal.

Limitations of TMB Assessment

The impact of tumor purity and intratumoral heterogeneity on the accuracy of TMB estimates was evaluated in a simulation experiment (FIG. 1). The experiment modeled two tumor samples with distinct subclonal composition, and assessed their estimated TMB at tumor purity levels ranging from 20% to 100% in 10% increments. The first simulated tumor with TMB of 265 contained four mutation clusters at cellular fractions 1.00 (n=100), 0.70 (n=50), 0.40 (n=40), and 0.2 (n=75). The second simulated tumor with TMB of 150 contained two mutation clusters at cellular fractions 1.00 (n=100), and 0.50 (n=50). At each level of tumor purity, the following process was repeated in 10 replicates to estimate the observed TMB. Distinct coverage (c) of each mutation was determined as:

c˜\dot{Γ}(βμ_{C},β)

where μ_{C} is the mean distinct coverage of the sample, and was set to set to 200. The rate parameter β determined the variance of base-level coverage in the sample, and was set to 0.013 based on evaluation of coverage distribution in 100 tumor samples. Distinct mutant read count (m) were generated by assuming a draw from a binomial distribution with probability of success set to the expected mutation allele frequency (Vₑₓₚ) given the purity of the tumor sample (α) and cellular fraction of the mutation (CT), assuming absence of somatic copy number alterations at the mutation loci as follows:

$${v}_{exp} = \frac{α * C F}{2}$$

$$m ∼ binom ( c , {v}_{exp} )$$

$$\hat{v} = \frac{m}{c}$$

Mutations with simulated distinct coverage c≥10, distinct mutant read count m≥3, and observed allele frequency \hat{ν}≥10% were determined to be present, and were tallied up to derive the observed TMB (obsTMB). The observed TMB was calculated in each replicate, and the median was reported (FIG. 4).

Correction of TMB for Tumor Purity

Corrected TMB (cTMB) values were generated based on observed TMB and tumor purity as follows. Given the findings that low tumor purity can limit the detection of subclonal mutations and skew the estimates of clonal composition, the level of intra-tumor heterogeneity in a set of TCGA NSCLC cancers with high tumor purity was first established. Purity, ploidy, and allele specific copy number profiles of the tumor samples based on analysis of SNP6 copy number array data were obtained from Synapse (synapse.org/#!Synapse:syn1710464.2). A set of 31 NSCLC samples with tumor purity of at least 80% and tumor ploidy in the range of [1.5, 5.0] was selected, where highly confident mutation calls (MC3 set) were available, and somatic copy number profile was determined. The cellular fraction of mutations in each tumor was estimated as described above, and determined the fraction of clonal mutations. This analysis revealed a low level of intra-tumor heterogeneity in untreated lung tumors, as it was observed clonal mutation fraction of 70% or above in all but two of the 31 tumors analyzed. Given the small number of lung tumors where the clonal composition could be accurately determined, an additional group of samples was identified to supplement the original set. 704 highly pure (purity >=80%) tumors were identified with available mutation and copy number data from the TCGA project in tumor types other than NSCLC, and characterized them in terms of clonal composition. An estimate was derived for the clonal composition of each tumor defined as the frequency of observed mutations in CF bins of width 0.05 spanning the [0,1] interval, and used these estimates as a basis to model mutation CF values in the simulation experiment. This set was further filtered to ensure that their level of intra-tumor heterogeneity matches that of NSCLC tumors by requiring clonal mutation fraction of 70% or above. The clonal composition from this reference combined set of NSCLC (n=29) and other (n=577) tumors with high clonal fraction (>=70%) was used to model mutation CF in the following simulation experiment.

20,000 in silico tumor samples were subsequently simulated, where the true TMB of each tumor was determined by sampling from the distribution of TMB in TCGA NSCLC samples. The mean sample sequence depth of coverage (C) was set to follow a normal distribution with μ=150 and σ=10. The clonal composition of each tumor was specified by randomly sampling from the reference set. The cancer cell fraction of mutations in each tumor were determined by sampling from a multinomial distribution with p parameters set to match the tumor's clonal composition.

Next, following the approach outlined above, the observed TMB (obsTMB) was determined at tumor purity values ranging from 10-100% for each tumor sample. At each level of tumor purity and for each tumor sample, the ratio of true to observed TMB was determined. The median of this ratio across the simulated tumors was considered as a multiplicative correction factor used to transform the observed TMB to a value referred to as corrected TMB (cTMB) that more closely approximates the true TMB. The median and 95% confidence interval of the correction factor (r) calculated at different levels of tumor purity (α) from the simulation experiment are reported (Table 4).

cTMB=r(α)*obsTMB

This approach was applied to the tumor samples in cohort 1 and estimated the corrected TMB and its 95% confidence interval (FIG. 4).

HLA Genetic Variation

OptiType v1.2. was used to determine HLA class I haplotypes as described elsewhere (see, e.g., Szolek et al., Bioinformatics 30:3310-3316 (2014)). The highly polymorphic nature of the HLA loci limits the accuracy of sequencing read alignment and somatic mutation detection by conventional methods. Therefore, a separate bioinformatic analysis using POLYSOLVER27 was applied to detect and annotate the somatic mutations in class I HLA genes. HLA class I haplotypes derived from application of Optitype-v1.2 to TCGA RNA-seq samples were retrieved from Genomic Data Commons (gdc.cancer.gov/about-data/publications/panimmune). To assess the possibility of loss of germline alleles in tumor, allele specific copy number profiles of the tumor samples from analysis of SNP6 copy number array data were obtained from Synapse (synapse.org/#!Synapse:syn1710464.2). Loss of heterozygosity of each HLA gene was determined by considering the minor allele copy number of the overlapping genomic region (minor CN=0 indicated complete loss of minor allele). Individual HLA-I alleles are classified into discrete supertypes, based upon similar peptideanchor-binding specificities as described elsewhere (see, e.g., Sidney et al., BMC immunology 9:1 (2008)).

Evaluation of Somatic HLA Loss

Given the essential role of MHC class I molecules in presentation of neo-antigens and initiation of a cascade of events that leads to anti-tumor immune response, we determined their maintenance or loss in tumor by applying LOHHLA using default program settings as described elsewhere (see, e.g., McGranahan et al., Cell 171:1259-1271 e1211 (2017)). LOHHLA determines allele specific copy number of HLA locus by realignment of NGS reads to patient-specific HLA reference sequences, and correction of the resulting coverage profile for tumor purity and ploidy. At each HLA locus heterozygous in germline, loss of heterozygosity was declared if the copy number for one of the two alleles was below 0.5, and there was a statistically significant different between the log copy ratio of the two alleles (PVal_unique <0.01). The unique number of class I HLA alleles in tumor was calculated by subtracting the number of germline heterozygous alleles with somatic LOH from the total number of unique alleles in germline.

TCR Sequencing

TCR clones were evaluated in tumor tissue by next generation sequencing. DNA from tumor samples was isolated by using the Qiagen DNA FFPE kit (Qiagen, CA). TCR-β CDR3 regions were amplified using the survey ImmunoSeq assay in a multiplex PCR method using 45 forward primers specific to TCR VP gene segments and 13 reverse primers specific to TCR Jβ gene segments (Adaptive Biotechnologies) as described elsewhere (see, e.g., Carlson et al., Nature communications 4:2680 (2013)). Productive TCR sequences were further analyzed. For each sample, a clonality metric was estimated in order to quantitate the extent of mono- or oligo-clonal expansion by measuring the shape of the clone frequency distribution as described elsewhere (see, e.g., Gao et al., Cell 167:397-404 e399 (2016)). Clonality values range from 0 to 1, where values approaching 1 indicate a nearly monoclonal population (Table 13).

TABLE 13
TCR-beta Sequencing Analysis.

| | | Total | Total | | Total | Productive | | |
|---|---|---|---|---|---|---|---|---|
| | Sample | Tem- | Productive | Fraction | Rearrange- | Rearrange- | Max Productive | Productive |
| Patient ID | Description | plates | Templates | Productive | ments | ments | Frequency | Clonality |
| CGLU111 | CGLU111T2 | 394 | 199 | 0.505076128 | 374 | 190 | 0.003128626 | 0.010050251 |
| CGLU115 | CGLU115T | 216 | 79 | 0.365740746 | 201 | 76 | 0.003216448 | 0.025316456 |
| CGLU116 | CGLU116T | 4248 | 3353 | 0.789312596 | 3609 | 2805 | 0.012799076 | 0.005666568 |
| CGLU117 | CGLU117T | 193 | 73 | 0.378238348 | 181 | 72 | 0.001215202 | 0.02739726 |
| CGLU120 | CGLU120T | 4065 | 3453 | 0.849446471 | 2280 | 1836 | 0.128506005 | 0.080220096 |
| CGLU121 | CGLU121T | 2744 | 2186 | 0.796647209 | 1978 | 1575 | 0.055913258 | 0.018370463 |
| CGLU124 | CGLU124T2 | 121 | 64 | 0.528925605 | 109 | 60 | 0.00539888 | 0.03125 |
| CGLU125 | CGLU125T | 2590 | 2085 | 0.805019315 | 1994 | 1567 | 0.031323135 | 0.012470024 |
| CGLU126 | CGLU126T1 | 1283 | 1044 | 0.813717859 | 1090 | 877 | 0.016258391 | 0.012452107 |
| CGLU127 | CGLU127T1 | 1240 | 869 | 0.700806433 | 1129 | 777 | 0.008627573 | 0.009205984 |
| CGLU128 | CGLU128T | 123 | 87 | 0.707317054 | 115 | 80 | 0.006312021 | 0.022988506 |
| CGLU129 | CGLU129T1 | 9461 | 7521 | 0.794947658 | 7734 | 6087 | 0.028438555 | 0.010238 |
| CGLU130 | CGLU130T | 740 | 605 | 0.817567545 | 665 | 539 | 0.009391389 | 0.011570248 |
| CGLU131 | CGLU131T2 | 3975 | 3111 | 0.782641488 | 2679 | 2131 | 0.069230579 | 0.039215688 |
| CGLU133 | CGLU133T | 158 | 101 | 0.639240489 | 147 | 97 | 0.006168455 | 0.03960396 |
| CGLU135 | CGLU135T | 6547 | 5330 | 0.814113312 | 4772 | 3813 | 0.042365704 | 0.018386491 |
| CGLU159 | CGLU159T | 839 | 680 | 0.810488655 | 545 | 438 | 0.162795544 | 0.138593718 |
| CGLU162 | CGLU162T | 918 | 622 | 0.677559894 | 809 | 530 | 0.025089854 | 0.040192924 |
| CGLU163 | CGLU163T | 412 | 302 | 0.733009689 | 373 | 273 | 0.006969676 | 0.009933775 |
| CGLU168 | CGLU168T1_3 | 4517 | 3658 | 0.809829511 | 2327 | 1863 | 0.164920613 | 0.101329111 |
| CGLU169 | CGLU169T | 16433 | 13434 | 0.817501347 | 12872 | 10507 | 0.018517194 | 0.005061783 |
| CGLU172 | CGLU172T | 916 | 742 | 0.810043646 | 705 | 558 | 0.02629278 | 0.026954178 |
| CGLU178 | CGLU178T | 41 | 13 | 0.317073162 | 36 | 11 | 0.019276058 | 0.15384616 |
| CGLU185 | CGLU185T1 | 127 | 73 | 0.574803134 | 98 | 53 | 0.053379722 | 0.12328767 |
| CGLU189 | CGLU189T | 74 | 29 | 0.391891881 | 68 | 26 | 0.01050014 | 0.068965517 |
| CGLU198 | CGLU198T | 16363 | 13568 | 0.829187779 | 7061 | 5592 | 0.134994894 | 0.044221699 |
| CGLU203 | CGLU203T | 132 | 86 | 0.651515134 | 129 | 85 | 0.000995726 | 0.023255814 |
| CGLU208 | CGLU208T | 17886 | 14235 | 0.795873846 | 15540 | 12319 | 0.013891555 | 0.003090973 |
| CGLU211 | CGLU211T | 336 | 236 | 0.702380933 | 298 | 209 | 0.013943339 | 0.021186441 |
| CGLU212 | CGLU212T | 92 | 50 | 0.543478246 | 88 | 49 | 0.001933075 | 0.039999999 |
| CGLU213 | CGLU213T | 2064 | 1626 | 0.787790676 | 1601 | 1251 | 0.035109852 | 0.020295203 |
| CGLU231 | CGLU231T | 3620 | 2846 | 0.786187824 | 2388 | 1888 | 0.042469516 | 0.016865777 |
| CGLU232 | CGLU232T | 641 | 484 | 0.755070182 | 532 | 402 | 0.021708163 | 0.02892562 |
| CGLU243 | CGLU243T_3 | 17281 | 13853 | 0.801631828 | 12414 | 9844 | 0.053106196 | 0.017252581 |
| CGLU244 | CGLU244T | 893 | 717 | 0.802911512 | 643 | 497 | 0.039763201 | 0.033472803 |
| CGLU246 | CGLU246T_3 | 1839 | 1481 | 0.805328961 | 1254 | 1005 | 0.060288221 | 0.030384876 |
| CGLU247 | CGLU247T_1 | 18602 | 15238 | 0.819159208 | 12406 | 9994 | 0.064126529 | 0.040687755 |
| CGLU262 | CGLU262T2_4 | 2332 | 1797 | 0.770583169 | 1184 | 931 | 0.128730372 | 0.04618809 |
| CGLU268 | CGLU268T_1 | 2223 | 1792 | 0.806117837 | 1794 | 1421 | 0.022926599 | 0.018415179 |
| CGLU270 | CGLU270T_2 | 1323 | 1040 | 0.786092193 | 1075 | 838 | 0.019186329 | 0.009615385 |
| CGLU287 | CGLU287T_1 | 851 | 652 | 0.766157441 | 742 | 569 | 0.014226519 | 0.018404909 |
| CGLU288 | CGLU288T_2 | 454 | 335 | 0.737885472 | 422 | 318 | 0.004738025 | 0.014925373 |
| CGLU289 | CGLU289T_5 | 5619 | 4363 | 0.776472661 | 3420 | 2625 | 0.063485354 | 0.022690808 |
| CGLU295 | CGLU295T_2 | 8441 | 6809 | 0.806657957 | 6034 | 4816 | 0.05393346 | 0.021442208 |
| CGLU299 | CGLU299T | 52337 | 41953 | 0.801593497 | 40539 | 32397 | 0.026405668 | 0.004171335 |
| CGLU304 | CGLU304T | 7175 | 5766 | 0.803623671 | 6028 | 4853 | 0.019148629 | 0.008151231 |
| CGLU307 | CGLU307T_1 | 10249 | 8236 | 0.803590572 | 5092 | 3965 | 0.126493752 | 0.054031082 |
| CGLU309 | CGLU309T | 11845 | 10399 | 0.87792315 | 2625 | 2059 | 0.29039818 | 0.08827772 |
| CGLU310 | CGLU310T | 1099 | 848 | 0.771610534 | 944 | 718 | 0.026097074 | 0.030660378 |
| CGLU329 | CGLU329T | 598 | 459 | 0.767558508 | 557 | 431 | 0.007541547 | 0.021786492 |
| CGLU334 | CGLU334T_1 | 67 | 32 | 0.477611948 | 65 | 32 | NE | 0.03125 |
| CGLU337 | CGLU337T | 819 | 645 | 0.787545766 | 634 | 494 | 0.035201941 | 0.04496124 |
| CGLU341 | CGLU341T_3 | 2791 | 2413 | 0.864564649 | 1510 | 1286 | 0.104942001 | 0.060505595 |
| CGLU348 | CGLU348T1 | 487 | 382 | 0.784394261 | 426 | 334 | 0.015035465 | 0.018324608 |
| CGLU389 | CGLU389T1_1 | 1820 | 1523 | 0.836813164 | 1331 | 1088 | 0.058203705 | 0.034799736 |
| CGLU510 | CGLU510T2 | 17868 | 14984 | 0.838594112 | 10503 | 8574 | 0.083714187 | 0.026895355 |
| CGLU512 | CGLU512T2 | 162 | 110 | 0.679012327 | 148 | 99 | 0.01322518 | 0.045454547 |
| CGLU514 | CGLU514T1 | 963 | 744 | 0.772585649 | 580 | 438 | 0.09330143 | 0.049731184 |
| CGLU515 | CGLU515T2 | 74 | 31 | 0.418918908 | 65 | 28 | 0.009715738 | 0.064516127 |
| CGLU519 | CGLU519T1 | 1011 | 823 | 0.814045477 | 833 | 672 | 0.020963168 | 0.012150669 |
| CGLU521 | CGLU521T1 | 6840 | 5501 | 0.804239744 | 3529 | 2721 | 0.124937966 | 0.047627702 |

Total templates refers to the sum of templates for all rearrangements in the sample,
total productive templates refers to the sum of templates for all productive rearrangements in the sample,
fraction productive denotes the fraction of productive templates among all templates,
productive rearrengements refer to the count of unique rearrangements in the sample that are in-frame and do not contain a stop codon,
Max productive frequency refers to the maximum productive frequency value found within a sample,
productive frequency denotes the frequency of a specific productive
rearrangement among all productive rearrangements within a sample.
Values for clonality range from 0 to 1, where values near 1 represent samples with one or a few predominant rearrangements
and clonality values near 0 represent more polyclonal samples.
T; tumor,
NE; non evaluable.

Immunohistochemistry and Interpretation of CD8 Staining

Immunolabeling for CD8 detection was performed on formalin-fixed, paraffin embedded sections on a Ventana Discovery Ultra autostainer (Roche Diagnostics). Briefly, following deparaffinization and rehydration, epitope retrieval was performed using Ventana Ultra CC1 buffer (Roche Diagnostics) at 96° C. for 64 minutes. Sections were subsequently incubated with the primary mouse anti-human CD8 antibody, (1:100 dilution, clone m7103, Dako) at 36° C. for 60 minutes, followed by incubation with an anti-mouse HQ detection system (Roche Diagnostics) and application of the Chromomap DAB IHC detection kit (Roche Diagnostics). A minimum of 100 tumor cells were evaluated per specimen. CD8-positive lymphocyte density was evaluated per 20× high power field.

Statistical Analyses

Differences between responding and non-responding tumors were evaluated using chi-square or Fisher's exact test for categorical variables and the Mann-Whitney test for continuous variables. The Pearson correlation coefficient (R) was used to assess correlations between continuous variables. P values were corrected using the Benjamini-Hochberg procedure and the associated false discovery rate (FDR) values were calculated. Tumors were classified based on their non-synonymous sequence alteration load in high and low mutators, using the second tertile as a cut-off point. The median point estimate and 95% CI for PFS and OS were estimated by the Kaplan-Meier method and survival curves were compared by using the nonparametric log rank test. Univariate Cox proportional hazards regression analysis was used to determine the impact of individual parameters on overall survival. A multivariable Cox proportional hazards model was employed using corrected TMB, RTK mutations, smoking mutational signature and number of HLA germline alleles. A risk score reflecting the relative hazard was calculated as the exponential of the sum of the product of mean-centered covariate values and their corresponding coefficient estimates for each case. The second tertile of the risk score was used to classify patients in high risk (top 33.3%) and low risk (bottom 66.6%) groups. All p values were based on two-sided testing and differences were considered significant at p<0.05. Statistical analyses were done using the SPSS software program (version 25.0.0 for Windows, IBM, Armonk, N.Y.) and R version 3.2 and higher, http://www.R-project.org/).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprises:
(a) identifying a sample from said mammal as having a mutation in an ARID1A nucleic acid sequence; and
(b) administering a cancer immunotherapy to said mammal under conditions wherein the number of cancer cells present within said mammal is reduced.

2. The method claim 1 wherein the sample is identified as having a molecular smoking signature.

3. The method of claim 1, wherein said sample comprises at least one cancer cell.

4. The method of claim 3, wherein said sample is a tissue sample.

5. A method for treating a mammal having cancer, wherein said method comprises:
administering a cancer immunotherapy to a mammal identified as having at least one cancer cell having a mutation in an ARID1A nucleic acid sequence.

6. The method of claim 5 wherein the mammal is identified as having at least one cancer cell with a molecular smoking signature.

7. The method of claim 1, wherein said mammal is a human.

8. The method of claim 1, wherein said cancer immunotherapy is selected from the group consisting of alemtuzumab, atezolizumab, avelumab, ipilimumab, ofatumumab, nivolumab, pembrolizumab, rituximab, and durvalumab.

9. The method of claim 1, wherein said mammal is further administered an additional cancer treatment.

10. The method of claim 9, wherein said additional cancer treatment is selected from the group consisting of surgery, radiation therapy, administration of a chemotherapy, administration of a hormone therapy, administration of a targeted therapy, and administration of a cytotoxic therapy.

11. A method for treating a mammal having cancer, wherein said method comprises:
(a) identifying a sample from said mammal as an activating mutation in EGFR nucleic acid, an activating mutation in ERBB2 nucleic acid, an activating mutation in MET nucleic acid, an activating mutation in FGFR1 nucleic acid, or an activating mutation in IGF1R nucleic acid; and
(b) administering a cancer treatment to said mammal under conditions wherein the number of cancer cells present within said mammal is reduced, wherein said cancer treatment is not a cancer immunotherapy;
or
A method for treating a mammal having cancer, wherein said method comprises:
(a) identifying a sample from said mammal as having germline homozygosity or a loss of at least one HLA class I locus; and
(b) administering a cancer treatment to said mammal under conditions wherein the number of cancer cells present within said mammal is reduced, wherein said cancer treatment is not a cancer immunotherapy;
or
A method for treating a mammal having cancer, wherein said method comprises:
(a) identifying a sample from said mammal as having a mutation in a KEAP1 nucleic acid sequence; and
(b) administering a cancer treatment to said mammal, wherein said cancer treatment is not a cancer immunotherapy.

12-13. (canceled)

14. The method of claim 1, wherein said sample comprises at least one cancer cell.

15. The method of claim 14, wherein said sample is a tissue sample.

16. A method for treating a mammal having cancer, wherein said method comprises:
administering a cancer treatment to a mammal identified as having at least one cancer cell having an activating mutation in EGFR nucleic acid, an activating mutation in ERBB2 nucleic acid, an activating mutation in MET nucleic acid, an activating mutation in FGFR1 nucleic acid, or an activating mutation in IGF1R nucleic acid, wherein said cancer treatment is not a cancer immunotherapy;
or
A method for treating a mammal having cancer, wherein said method comprises:
administering a cancer treatment to a mammal identified as having germline homozygosity or a loss of at least one HLA class I locus, wherein said cancer treatment is not a cancer immunotherapy;
or
A method for treating a mammal having cancer, wherein said method comprises:
administering a cancer treatment to a mammal identified as having a mutation in a KEAP1 nucleic acid sequence, wherein said cancer treatment is not a cancer immunotherapy.

17-18. (canceled)

19. The method of claim 1, wherein said mammal is a human.

20. The method of claim 1, wherein said cancer treatment is selected from the group consisting of surgery, radiation therapy, administration of a chemotherapy, administration of a hormone therapy, administration of a targeted therapy, and administration of a cytotoxic therapy.

21. A method for identifying a mammal as having a cancer that is likely to respond to an immunotherapy, said method comprising:
(a) determining a corrected tumor mutation burden (cTMB) of said cancer;
(b) determining a mutational signature of said cancer; and
identifying said cancer as not being likely to respond to said immunotherapy when said mutational signature of said cancer comprises i) an activating mutation in a nucleic acid encoding a receptor tyrosine kinase (RTK) polypeptide; and ii) germline homozygosity or a loss of at least one HLA class I locus;
or
A method for identifying a mammal as having a cancer that is likely to respond to an immunotherapy, said method comprising:
(a) determining a corrected tumor mutation burden (cTMB) of said cancer;
(b) determining a mutational signature of said cancer; and
identifying said cancer as being likely to respond to said immunotherapy when said mutational signature of said cancer comprises i) mutation in an ARID1A nucleic acid sequence or a molecular smoking signature; and ii) germline heterozygosity at least one HLA class I locus;
or
A method for determining a cTMB, said method comprising:
determining an observed TMB (obsTMB) of a sample comprising at least one cancer cell;
determining a tumor purity (a) of said sample; and
adjusting said observed TMB based on said tumor purity using a correction factor (r) as set forth in Table 4 to determine the cTMB.

22. The method of claim 21, wherein said nucleic acid encoding said RTK polypeptide is a EGFR, ERBB2, MET, FGFR1, or IGF1R nucleic acid.

23. (canceled)

24. The method of claim 21, wherein said molecular smoking signature comprises cytosine (C) to adenosine (A) transversions (C>A transversions).

25. The method of claim 21, wherein said determining said cTMB of said cancer comprises:
determining an observed TMB (obsTMB) of a sample comprising at least one cancer cell from said cancer;
determining a tumor purity (a) of said sample; and
adjusting said observed TMB based on said tumor purity using a correction factor (r) as set forth in Table 4 to determine the cTMB.

26-30. (canceled)
